(19) United States
(12) Patent Application Publication
(10) Pub. No.: US 2020/0247851 A1
(43) Pub. Date: Aug. 6, 2020

(54) Methods For Generating Epitopes For Binding To Recognition Molecules By Templated Assembly

(71) Applicant: TriBiotica LLC, Madison, WI (US)

(72) Inventors: Ian Dunn, Madison, WI (US); Matthew Lawler, Madison, WI (US)

(21) Appl. No.: 16/637,858

(22) PCT Filed: Aug. 10, 2018
(86) PCT No.: PCT/US2018/046179

§ 371 Date: Feb. 10, 2020

Related U.S. Application Data
(60) Provisional application No. 62/544,010, filed on Aug. 11, 2017.

(51) Int. Cl. C07K 14/00; C12N 15/115

(52) U.S. Cl. CPC C07K 14/001; C12N 2310/16; C12N 15/115

(57) ABSTRACT

The present disclosure provides polypeptides and polypeptide-nucleic acid conjugates comprising portions of epitopes, and methods of using target molecule binding components, such as aptamers, to present template sequences, where the target molecule binding components bind to target molecules unique to specific cellular targets, for the purpose of templated assembly of the epitopes for recognition molecules.

### FIELD

The present disclosure is directed, in part, to polypeptides and polypeptide-nucleic acid conjugates comprising portions of epitopes, and methods of using target molecule binding components to present template sequences, where the target molecule binding components bind to target molecules unique to specific cellular targets, for the purpose of templated assembly of the epitopes for recognition molecules.

### BACKGROUND

A goal of drug development is delivering potent bio-therapeutic interventions to pathogenic cells, such as virus infected cells, neoplastic cells, cells producing an autoimmune response, and other dysregulated or dysfunctional cells. Examples of potent bio-therapeutic interventions capable of combating pathogenic cells include toxins, pro-apoptotic agents, and immunotherapy approaches that re-direct immune cells to eliminate pathogenic cells. Unfortunately, developing these agents is extremely difficult because of the high risk of toxicity to adjacent normal cells or the overall health of the patient.

A method that has emerged to allow delivery of potent interventions to pathogenic cells while mitigating toxicity to normal cells is targeting of therapeutics by directing them against molecular markers specific for pathogenic cells. Targeted therapeutics have shown extraordinary clinical results in restricted cases, but are currently limited in their applicability due to a lack of accessible markers for targeted therapy. It is extremely difficult, and often impossible, to discover protein markers for many pathogenic cell types.

More recently, therapies targeted to nucleic acid targets specific to pathogenic cells have been developed. Existing nucleic acid-targeted therapies, such as siRNA, are able to down-modulate expression of potentially dangerous genes, but do not deliver potent cytotoxic or cytostatic interventions and thus are not particularly efficient at eliminating the dangerous cells themselves. Hence, there exists a need to combat the poor efficacy and/or severe side effects of existing bio-therapeutic interventions.

Finding proximal binding sites in proteins or other macromolecules cannot be performed according to simple hybridization rules. Rather than a readily applied digital code, the ligand binding can be seen as an analog process, where the ligand and its receptor pocket share a shape-based complementarity. Rational design of such ligand-mediated templating therefore requires detailed three-dimensional structural information. Even where crystal structures of proteins (considered as possible target templates) is available, design of interactive ligands is another step upward in difficulty, especially where such ligands must bind within tightly proscribed spatial boundaries relative to each other. Moreover, such design must also take into account the possibility of binding-related conformational changes (akin to allostery), which could inadvertently destroy the desired spatial proximity. While these caveats do not rule out testing specific protein choices for templating purposes, they do emphasize the difficulties of finding non-nucleic acid templates in target aberrant cells in realistic time-frames.

Although much progress has been made in recent years with respect to therapy for specific cancers, a great many therapeutic gaps still exist. Such unmet needs for better treatments are highly applicable to many tumor types. Moreover, a general therapy capable of targeting specific pathological or undesirable cells is desired.

### SUMMARY

Among all possible recognition molecules, any monoclonal antibody recognizing a defined epitope, for example, could be used for devising a split-epitope click assembly strategy. In practice, important considerations in making such a choice include the levels of structural information available and the availability of an antibody or other recognition molecule. The recognition of HER-2 (erb-B2; expressed in certain tumors, but particularly in a subset of breast cancer) by trastuzumab (HERCEPTIN®) is suitable. The structure of trastuzumab in complex with HER-2 has been elucidated, and HERCEPTIN® has proven effective as an oncotherapeutic.

In general, the present disclosure provides isolated polypeptides comprising the formula: SerGlyGlyGlySerGlyGlyGlyGlnLeuXaa¹ProTyrGluXaa²TrpGluLeuXaa³His, wherein one of: Xaa¹ is Cys, Xaa² is Leu, and Xaa³ is Ser (SEQ ID NO:1); Xaa¹ is Gly, Xaa² is Cys, and Xaa³ is Ser (SEQ ID NO:2); or Xaa¹ is Gly, Xaa² is Leu, and Xaa³ is Cys (SEQ ID NO:3).

The present disclosure also provides isolated polypeptides comprising the formula: SerGlyGlyGlySerGlyGlyGlyGlnXaa¹LeuXaa²GlyXaa³ProXaa⁴TyrXaa⁵GluXaa⁶LeuXaa⁷TrpXaa⁸GluXaa⁹LeuXaa¹⁰SerXaa¹¹His, wherein one of: Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent (SEQ ID NO:4); Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent (SEQ ID NO:5); Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent (SEQ ID NO:6); Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent (SEQ ID NO:7); Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent (SEQ ID NO:8); Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent (SEQ ID NO:9); Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent (SEQ ID NO:10); Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent (SEQ ID NO:11); Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are absent (SEQ ID NO:12); Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent (SEQ ID NO:13); or Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent (SEQ ID NO:14).

The present disclosure also provides compositions comprising a pair of polypeptides, wherein the pair of polypeptides is: a) SerGlyGlyGlySerGlyGlyGlyGlnLeu (SEQ ID NO:15) and Xaa¹ProTyrGluXaa²TrpGluLeuXaa³His (SEQ ID NO:16), wherein Xaa¹ is Cys, Xaa² is Leu, and Xaa³ is Ser; b) SerGlyGlyGlySerGlyGlyGlyGlnLeuXaa¹ProTyrGlu (SEQ ID NO:17) and Xaa²TrpGluLeuXaa³His (SEQ ID NO:18), wherein Xaa¹ is Gly, Xaa² is Cys, and Xaa³ is Ser; or c) SerGlyGlyGlySerGlyGlyGlyGlnLeuXaa¹ProTyrGluXaa²TrpGluLeu (SEQ ID NO:19) and Xaa³His, wherein Xaa¹ is Gly, Xaa² is Leu, and Xaa³ is Cys.

The present disclosure also provides compositions comprising a pair of polypeptides, wherein the pair of polypeptides is: a) SerGlyGlyGlySerGlyGlyGlyGln (SEQ ID NO:20) and Xaa¹LeuXaa²GlyXaa³ProXaa⁴TyrXaa⁵GluXaa⁶LeuXaa⁷TrpXaa⁸GluXaa⁹LeuXaa¹⁰SerXaa¹¹His (SEQ ID NO:21), wherein Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent; b) SerGlyGlyGlySerGlyGlyGlyGlnXaa¹Leu (SEQ ID NO:15) and Xaa²GlyXaa³ProXaa⁴TyrXaa⁵GluXaa⁶LeuXaa⁷TrpXaa⁸GluXaa⁹LeuXaa¹⁰SerXaa¹¹His (SEQ ID NO:22), wherein Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent; c) SerGlyGlyGlySerGlyGlyGlyGlnXaa¹LeuXaa²Gly (SEQ ID NO:23) and Xaa³ProXaa⁴TyrXaa⁵GluXaa⁶LeuXaa⁷TrpXaa⁸GluXaa⁹LeuXaa¹⁰SerXaa¹¹His (SEQ ID NO:16), wherein Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent; d) SerGlyGlyGlySerGlyGlyGlyGlnXaa¹LeuXaa²GlyXaa³Pro (SEQ ID NO:24) and Xaa⁴TyrXaa⁵GluXaa⁶LeuXaa⁷TrpXaa⁸GluXaa⁹LeuXaa¹⁰SerXaa¹¹His (SEQ ID NO:25), wherein Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent; e) SerGlyGlyGlySerGlyGlyGlyGlnXaa¹LeuXaa²GlyXaa³ProXaa⁴Tyr (SEQ ID NO:26) and Xaa⁵GluXaa⁶LeuXaa⁷TrpXaa⁸GluXaa⁹LeuXaa¹⁰SerXaa¹¹His (SEQ ID NO:27), wherein Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent; f) SerGly GlyGlySerGlyGlyGlyGlnXaa¹LeuXaa²GlyXaa³ProXaa⁴TyrXaa⁵Glu (SEQ ID NO:17) and Xaa⁶LeuXaa⁷TrpXaa⁸GluXaa⁹LeuXaa¹⁰SerXaa¹¹His (SEQ ID NO:28), wherein Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent; g) SerGlyGlyGly SerGlyGlyGlyGlnXaa¹LeuXaa²GlyXaa³ProXaa⁴TyrXaa⁵GluXaa⁶Leu (SEQ ID NO:29) and Xaa⁷TrpXaa⁸GluXaa⁹LeuXaa¹⁰SerXaa¹¹His (SEQ ID NO:18), wherein Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent; h) SerGlyGlyGlySerGly GlyGlyGlnXaa¹LeuXaa²GlyXaa³ProXaa⁴TyrXaa⁵GluXaa⁶LeuXaa⁷Trp (SEQ ID NO:30) and Xaa⁸GluXaa⁹LeuXaa¹⁰SerXaa¹¹His (SEQ ID NO:31), wherein Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent; i) SerGlyGlyGlySerGlyGlyGlyGln Xaa¹LeuXaa²GlyXaa³ProXaa⁴TyrXaa⁵GluXaa⁶LeuXaa⁷TrpXaa⁸Glu (SEQ ID NO:32) and Xaa⁹LeuXaa¹⁰SerXaa¹¹His (SEQ ID NO:33), wherein Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are absent; j) SerGlyGlyGlySerGlyGlyGlyGlnXaa¹Leu Xaa²GlyXaa³ProXaa⁴TyrXaa⁵GluXaa⁶LeuXaa⁷TrpXaa⁸GluXaa⁹Leu (SEQ ID NO:19) and Xaa¹⁰SerXaa¹¹His, wherein Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent; or k) SerGlyGlyGlySerGlyGlyGlyGlnXaa¹LeuXaa²GlyXaa³Pro Xaa⁴TyrXaa⁵GluXaa⁶LeuXaa⁷TrpXaa⁸GluXaa⁹LeuXaa¹⁰Ser (SEQ ID NO:34) and Xaa¹¹His, wherein Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent.

The present disclosure also provides methods for the directed assembly of an epitope on a target cell for a recognition molecule comprising: a) contacting the target cell with a target molecule binding component, wherein the target molecule binding component comprises: i) a first portion that is able to bind to a target molecule on the surface of the target cell; and ii) second portion comprising a nucleic acid molecule linked to the first portion at either the 3′ or 5′ terminal end of the second portion; and b) contacting the target cell with a first epitope haplomer and a second epitope haplomer; wherein the first epitope haplomer comprises: i) a nucleic acid molecule that is complementary to the second portion of the target molecule binding component; and ii) a reactive effector moiety that is a first portion of the epitope; wherein the second epitope haplomer comprises: i) a nucleic acid molecule that is complementary to the second portion of the target molecule binding component; and a reactive effector moiety that is a second portion of the epitope; wherein the nucleic acid molecule of the first epitope haplomer is complementary to a region of the second portion of the target molecule binding component that is in spatial proximity to the region of the second portion of the target molecule binding component to which the nucleic acid molecule of the second epitope haplomer is complementary; and wherein the reactive effector moiety of the first epitope haplomer is in spatial proximity to the reactive effector moiety of the second epitope haplomer, thereby resulting in the directed assembly of the epitope.

The present disclosure also provides methods for the directed assembly of an epitope on a target cell for a recognition molecule comprising: a) contacting the target cell with a singlet aptamer, wherein the singlet aptamer comprises: i) a first portion folded into a tertiary structure that is able to bind to a target molecule on the surface of the target cell; and ii) a second portion comprising a nucleic acid molecule linked to the first portion at either the 3′ or 5′ terminal end of the second portion; and b) contacting the target cell with a first epitope haplomer and a second epitope haplomer; wherein the first epitope haplomer comprises: i) a nucleic acid molecule that is complementary to the second portion of the singlet aptamer; and ii) a reactive effector moiety that is a first portion of the epitope; wherein the second epitope haplomer comprises: i) a nucleic acid molecule that is complementary to the second portion of the singlet aptamer; and ii) a reactive effector moiety that is a second portion of the epitope; wherein the nucleic acid molecule of the first epitope haplomer is complementary to a region of the second portion of the singlet aptamer that is: in spatial proximity to the region of the second portion of the singlet aptamer to which the nucleic acid molecule of the second epitope haplomer is complementary; and wherein the reactive effector moiety of the first epitope haplomer is in spatial proximity to the reactive effector moiety of the second epitope haplomer, thereby resulting in the directed assembly of the epitope.

The present disclosure also provides methods for the directed assembly of an epitope on a target cell for a recognition molecule comprising: a) contacting the target cell with a dual proximal aptamer pair, wherein the dual proximal aptamer pair comprises a first aptamer and a second aptamer, wherein: the first aptamer comprises: i) a first portion folded into a tertiary structure that is able to bind to a target molecule on the surface of the target cell; and a second portion comprising a nucleic acid molecule linked to the first portion at either the 3′ or 5′ terminal end of the second portion; and the second aptamer comprises: i) a first portion folded into a tertiary structure that is able to bind to a target molecule on the surface of the target cell; and ii) a second portion comprising a nucleic acid molecule linked to the first portion at either the 3′ or 5′ terminal end of the second portion; and b) contacting the target cell with a first epitope haplomer and a second epitope haplomer; wherein the first epitope haplomer comprises: i) a nucleic acid molecule that is complementary to the second portion of the first aptamer; and ii) a reactive effector moiety that is a first portion of the epitope, wherein the second epitope haplomer comprises: i) a nucleic acid molecule that is complementary to the second portion of the second aptamer, and ii) a reactive effector moiety that is a second portion of the epitope; wherein the nucleic acid molecule of the first epitope haplomer is complementary to a region of the second portion of the first aptamer that is in spatial proximity to the region of the second portion of the second aptamer to which the nucleic acid molecule of the second epitope haplomer is complementary; and wherein the reactive effector moiety of the first epitope haplomer is in spatial proximity to the reactive effector moiety of the second epitope haplomer, thereby resulting in the directed assembly of the epitope.

The present disclosure also provides methods for the directed assembly of an epitope on a target cell for a recognition molecule comprising: a) contacting the target cell with a binary aptamer, wherein the binary aptamer comprises: i) a first portion folded into a tertiary structure that is able to bind to a target molecule on the surface of the target cell; ii) a second portion folded into a tertiary structure that is able to hind to a target molecule on the surface of the target cell; and iii) a third portion comprising a nucleic acid molecule located between the first and second portion; and b) contacting the target cell with a first epitope haplomer and a second epitope haplomer; wherein the first epitope haplomer comprises: i) a nucleic acid molecule that is complementary to the third portion of the binary aptamer; and ii) a reactive effector moiety that is a first portion of the epitope; wherein the second epitope haplomer comprises: i) a nucleic acid molecule that is complementary to the third portion of the binary aptamer; and ii) a reactive effector moiety that is a second portion of the epitope; wherein the nucleic acid molecule of the first epitope haplomer is complementary to a region of the third portion of the binary aptamer that is in spatial proximity to the region of the third portion of the binary aptamer to which the nucleic acid molecule of the second epitope haplomer is complementary; and wherein the reactive effector moiety of the first epitope haplomer is in spatial proximity to the reactive effector moiety of the second epitope haplomer, thereby resulting in the directed assembly of the epitope.

### BRIEF DESCRIPTION OF THE DRAWINGS

FIG. 1 shows a representative flow analysis of hybridization of a bilabeled probe sequence to a surface template positioned on a cell surface by means of a primary biotinylated antibody and a streptavidin bridge.

FIG. 2 shows a representative flow analysis of placement of a trastuzumab mimotope on a HER-2-negative tumor cell, compared to a HER-2+ breast cancer cell line control.

FIG. 3 shows a representative preparation of oligonucleotide-peptide conjugates (Oligo #408 (SEQ ID NO:130) bound to CLJ peptide (SEQ ID NO:132); Oligo #417 (SEQ ID NO:131) bound to JLC peptide (SEQ ID NO:133)) via cross-linking of —SH groups on both molecules by means of a bis-maleimide (PEG)₂ (BMP2) compound, and demonstration of conjugate formation on a denaturing 15% urea gel, with varying peptide-oligonucleotide combinations and reaction conditions.

FIG. 4 shows a representative ELISA example with a dilution series of mimotope for trastuzumab.

FIG. 5 shows the results of an ELISA assay using bioinylated unmodified mimotope (Bio-SGGGSGGGQLGPYELWELSH; SEQ ID NO:35) and a corresponding cysteine-modified mimotope (Bio-SGGGSGGGQLGPYELWELCH; SEQ ID NO:3).

### DESCRIPTION OF EMBODIMENTS

Assembly of functional epitopes from non-functional precursors in situ on a cell surface has the potential to convert an unresponsive pathogenic cell into a target recognized by a recognition molecule, such as an antibody, of interest. For this technology to be rendered feasible, an epitope must be severed into two segments such that it can be reconstituted when the participating fragments are brought into close spatial proximity by a templating process. Examples of templating processes are set forth in, for example, PCT Publication No. WO 14/197547. Where epitope segments are individually inert, but active as ligands for their respective recognition molecules only when assembled on a desired target cell, the potential for toxic and interfering bystander reactions in a therapeutic context is greatly reduced.

The target molecule binding components described herein can be any molecule that is able to bind to a target molecule on a target cell. In some embodiments, the target molecule binding component is an antibody that recognizes a target molecule on the surface of a target cell. In some embodiments, the target molecule binding component is a ligand, such as a peptide ligand, that recognizes a target molecule on the surface of a target cell. In some embodiments, the target molecule binding component is an aptamer that recognizes a target molecule on the surface of a target cell. In some embodiments, the aptamer is a singlet aptamer, a dual proximal aptamer pair, or a binary aptamer.

In embodiments where the target molecule binding component is a peptide ligand or antibody, the peptide ligand or antibody comprises: i) a first portion that is able to bind to a target molecule on the surface of the target cell; and ii) a second portion comprising a nucleic acid molecule linked to the first portion at either the 3′ or 5′ terminal end of the second portion. The method further comprises contacting the target cell with a first epitope haplomer and a second epitope haplomer. The first epitope haplomer comprises: i) a nucleic acid molecule that is complementary to the second portion of the peptide ligand or antibody; and ii) a reactive effector moiety that is a first portion of the epitope. The second epitope haplomer comprises: i) a nucleic acid molecule that is complementary to the second portion of the peptide ligand or antibody; and ii) a reactive effector moiety that is a second portion of the epitope. In this embodiment, the nucleic acid molecule of the first epitope haplomer is complementary to a region of the second portion of the peptide ligand or antibody that is in spatial proximity to the region of the second portion of the peptide ligand or antibody to which the nucleic acid molecule of the second epitope haplomer is complementary. In this embodiment, the reactive effector moiety of the first epitope haplomer is in spatial proximity to the reactive effector moiety of the second epitope haplomer, thereby resulting in the directed assembly of the epitope. The first and second haplomers are described below (in the context of using aptamers, but can also be used with any target molecule binding components, such as ligands, peptide ligands and antibodies) in more detail.

In some embodiments, the first portion of the peptide ligand that is able to bind to a target molecule comprises from about 5 amino acids to about 50 amino acids, from about 5 amino acids to about 40 amino acids, from about 5 amino acids to about 30 amino acids, from about 5 amino acids to about 20 amino acids, from about 5 amino acids to about 10 amino acids, from about 10 amino acids to about 50 amino acids, from about 10 amino acids to about 40 amino acids, from about 10 amino acids to about 30 amino acids, from about 10 amino acids to about 20 amino acids, from about 20 amino acids to about 50 amino acids, from about 20 amino acids to about 40 amino acids, from about 20 amino acids to about 30 amino acids, from about 30 amino acids to about 50 amino acids, or from about 30 amino acids to about 40 amino acids. In some embodiments, the first portion of the peptide ligand that is able to bind to a target molecule comprises from about 10 amino acids to about 30 amino acids.

The present disclosure provides methods for the directed assembly of an epitope on a target cell using a singlet aptamer, wherein the epitope is recognized and is able to interact or bind to a recognition molecule. In this embodiment, the method comprises contacting a target cell with a singlet aptamer. The singlet aptamer comprises: i) a first portion folded into a tertiary structure that is able to bind to a target molecule on the surface of the target cell; and ii) a second portion comprising a nucleic acid molecule linked to the first portion at either the 3′ or 5′ terminal end of the second portion. The method further comprises contacting the target cell with a first epitope haplomer and a second epitope haplomer. The first epitope haplomer comprises: i) a nucleic acid molecule that is complementary to the second portion of the singlet aptamer; and ii) a reactive effector moiety that is a first portion of the epitope. The second epitope haplomer comprises: i) a nucleic acid molecule that is complementary to the second portion of the singlet aptamer; and ii) a reactive effector moiety that is a second portion of the epitope. In this embodiment, the nucleic acid molecule of the first epitope haplomer is complementary to a region of the second portion of the singlet aptamer that is in spatial proximity to the region of the second portion of the singlet aptamer to which the nucleic acid molecule of the second epitope haplomer is complementary. In this embodiment, the reactive effector moiety of the first epitope haplomer is in spatial proximity to the reactive effector moiety of the second epitope haplomer, thereby resulting in the directed assembly of the epitope.

In some embodiments, the first portion of the singlet aptamer that is folded into a tertiary structure that is able to bind to a target molecule is a nucleic acid molecule. In some embodiments, the nucleic acid molecule that is the first portion of the singlet aptamer comprises from about 20 nucleotides to about 150 nucleotides, from about 20 nucleotides to about 120 nucleotides, from about 20 nucleotides to about 100 nucleotides, from about 20 nucleotides to about 80 nucleotides, or from about 20 nucleotides to about 60 nucleotides. In some embodiments, the nucleic acid molecule that is the first portion of the singlet aptamer comprises from about 20 nucleotides to about 80 nucleotides. In some embodiments, the nucleic acid molecule that is the first portion of the singlet aptamer comprises from about 40 nucleotides to about 60 nucleotides. In some embodiments, the nucleic acid molecule that is the first portion of the singlet aptamer has a Tm from about 45° to about 65° C. In some embodiments, the nucleic acid molecule that is the first portion of the singlet aptamer has a Tm from about 45° to about 55° C. In some embodiments, the nucleic acid molecule that is the first portion of the singlet aptamer has a Tm from about 55° to about 65° C. In some embodiments, the first portion of the singlet aptamer that is folded into a tertiary structure comprises either the 3′ or 5′ terminal end of the aptamer. In some embodiments, the first portion of the singlet aptamer that is folded into a tertiary structure comprises the 3′ terminal end of the aptamer. In some embodiments, the first portion of the singlet aptamer that is folded into a tertiary structure comprises the 5′ terminal end of the aptamer.

The singlet aptamer also comprises a second portion that comprises either the 3′ or 5′ terminal end of the aptamer (i.e., whichever terminal end is not a part of the first portion). Thus, in some embodiments, the first portion that is folded into a tertiary structure that is able to bind to a target molecule comprises the 5′ portion of the aptamer, leaving the second portion to comprise the 3′ terminal end of the aptamer. Alternately, the first portion that is folded into a tertiary structure that is able to bind to a target molecule can comprises the 3′ portion of the aptamer, leaving the second portion to comprise the 5′ terminal end of the aptamer.

The second portion of the singlet aptamer comprises a nucleic acid molecule. In some embodiments, the second portion of the singlet aptamer comprises from about 30 nucleotides to about 100 nucleotides, from about 30 nucleotides to about 90 nucleotides, from about 30 nucleotides to about 80 nucleotides, from about 30 nucleotides to about 70 nucleotides, from about 30 nucleotides to about 60 nucleotides, from about 30 nucleotides to about 50 nucleotides, from about 30 nucleotides to about 40 nucleotides, from about 40 nucleotides to about 100 nucleotides, from about 40 nucleotides to about 90 nucleotides, from about 40 nucleotides to about 80 nucleotides, from about 40 nucleotides to about 70 nucleotides, from about 40 nucleotides to about 60 nucleotides, from about 40 nucleotides to about 50 nucleotides, from about 50 nucleotides to about 100 nucleotides, from about 50 nucleotides to about 90 nucleotides, from about 50 nucleotides to about 80 nucleotides, from about 50 nucleotides to about 70 nucleotides, from about 50 nucleotides to about 60 nucleotides, from about 60 nucleotides to about 100 nucleotides, from about 60 nucleotides to about 90 nucleotides, from about 60 nucleotides to about 80 nucleotides, from about 60 nucleotides to about 70 nucleotides, from about 70 nucleotides to about 100 nucleotides, from about 70 nucleotides to about 90 nucleotides, from about 70 nucleotides to about 80 nucleotides, from about 80 nucleotides to about 100 nucleotides, from about 80 nucleotides to about 90 nucleotides. In some embodiments, the second portion of the singlet aptamer comprises from about 30 nucleotides to about 60 nucleotides.

In some embodiments, both the first portion and the second portion of the singlet aptamer comprise sequence regions that serve as primer binding sites for amplification purposes. In some embodiments, the 5′ terminal region of the singlet aptamer contains a first sequence region that serves as a first primer binding site for amplification purposes. In some embodiments, the 3′ terminal region of the singlet aptamer contains a second sequence region that serves as a second primer binding site for amplification purposes. Using both amplification primer binding sites in conjunction with the appropriate primers allows for amplification, such as by PCR, of the singlet aptamer. In some embodiments, the respective primer binding regions in the second portion of the singlet aptamer can also form part of the template regions for producing the functional epitope upon templated assembly.

The first epitope haplomer comprises a nucleic acid molecule that is complementary to the second portion of the singlet aptamer, and a reactive effector moiety that is a first portion of the epitope. In some embodiments, the 5′ end of the nucleic acid molecule of the first epitope haplomer is conjugated to the reactive effector moiety. In some embodiments where the reactive effector molecule is a peptide (e.g., the peptide is a first portion of the epitope), the 5′ end of the nucleic acid molecule of the first epitope haplomer is conjugated to the N-terminus of the peptide.

The second epitope haplomer also comprises a nucleic acid molecule that is complementary to the second portion of the singlet aptamer, and a reactive effector moiety that is a second portion of the epitope. In some embodiments, the 3′ end of the nucleic acid molecule of the second epitope haplomer is conjugated to the reactive effector moiety. In some embodiments where the reactive effector molecule is a peptide (e.g., the peptide is a second portion of the epitope), the 3′ end of the nucleic acid molecule of the second epitope haplomer is conjugated to the C-terminus of the peptide.

The nucleic acid molecule of the first epitope haplomer is complementary to a region of the second portion of the singlet aptamer. The nucleic acid molecule of the second epitope haplomer is also complementary to a region of the second portion of the singlet aptamer. The region of the second portion of the singlet aptamer to which the nucleic acid molecule of the first epitope haplomer is complementary is 5′ (referring to the second portion of the singlet aptamer) compared to the region of the second portion of the singlet aptamer to which the nucleic acid molecule of the second epitope haplomer is complementary. In some embodiments, the nucleic acid molecules of the first epitope haplomer and the second epitope haplomer each, independently, comprises from about 10 nucleotides to about 30 nucleotides, from about 10 nucleotides to about 25 nucleotides, from about 10 nucleotides to about 20 nucleotides, from about 10 nucleotides to about 18 nucleotides, or from about 10 nucleotides to about 15 nucleotides. In some embodiments, the nucleic acid molecules of the first epitope haplomer and the second epitope haplomer each, independently, comprises from about 6 nucleotides to about 24 nucleotides, from about 8 nucleotides to about 20 nucleotides, or from about 10 nucleotides to about 18 nucleotides.

In some embodiments, the region of the second portion of the singlet aptamer between which the nucleic acid molecule of the first epitope haplomer is complementary and the nucleic acid molecule of the second epitope haplomer is complementary comprises from about 18 nucleotides to about 100 nucleotides, from about 18 nucleotides to about 90 nucleotides, from about 18 nucleotides to about 80 nucleotides, from about 18 nucleotides to about 70 nucleotides, from about 18 nucleotides to about 60 nucleotides, from about 18 nucleotides to about 50 nucleotides, from about 18 nucleotides to about 40 nucleotides, from about 18 nucleotides to about 30 nucleotides, from about 18 nucleotides to about 25 nucleotides, from about 20 nucleotides to about 100 nucleotides, from about 20 nucleotides to about 90 nucleotides, from about 20 nucleotides to about 80 nucleotides, from about 20 nucleotides to about 70 nucleotides, from about 20 nucleotides to about 60 nucleotides, from about 20 nucleotides to about 50 nucleotides, from about 20 nucleotides to about 40 nucleotides, from about 20 nucleotides to about 30 nucleotides, from about 30 nucleotides to about 100 nucleotides, from about 30 nucleotides to about 90 nucleotides, from about 30 nucleotides to about 80 nucleotides, from about 30 nucleotides to about 70 nucleotides, from about 30 nucleotides to about 60 nucleotides, from about 30 nucleotides to about 50 nucleotides, from about 30 nucleotides to about 40 nucleotides, from about 40 nucleotides to about 100 nucleotides, from about 40 nucleotides to about 90 nucleotides, from about 40 nucleotides to about 80 nucleotides, from about 40 nucleotides to about 70 nucleotides, from about 40 nucleotides to about 60 nucleotides, from about 40 nucleotides to about 50 nucleotides, from about 50 nucleotides to about 100 nucleotides, from about 50 nucleotides to about 90 nucleotides, from about 50 nucleotides to about 80 nucleotides, from about 50 nucleotides to about 70 nucleotides, from about 50 nucleotides to about 60 nucleotides, from about 60 nucleotides to about 100 nucleotides, from about 60 nucleotides to about 90 nucleotides, from about 60 nucleotides to about 80 nucleotides, from about 60 nucleotides to about 70 nucleotides, from about 70 nucleotides to about 100 nucleotides, from about 70 nucleotides to about 90 nucleotides, from about 70 nucleotides to about 80 nucleotides, from about 80 nucleotides to about 100 nucleotides, from about 80 nucleotides to about 90 nucleotides, or from about 90 nucleotides to about 100 nucleotides. In some embodiments, the region of the second portion of the singlet aptamer between which the nucleic acid molecule of the first epitope haplomer is complementary and the nucleic acid molecule of the second epitope haplomer is complementary comprises from about 18 nucleotides to about 25 nucleotides.

The spacing of the regions of complementarity between the second portion of the singlet aptamer and the nucleic acid molecules of the first and second epitope haplomers (i.e., spatial proximity) results in the reactive effector moiety of the first epitope haplomer being in spatial proximity to the reactive effector moiety of the second epitope haplomer. As a result of the spatial proximity between the two reactive effector moieties, the directed assembly of the epitope is accomplished. The reactive effector moiety of the first epitope haplomer and the reactive effector moiety of the second epitope haplomer are in spatial proximity when a chemical reaction (such as any one of the chemical reactions described herein) can occur between the respective reactive effector moieties such that the two reactive effector moieties are joined to form the desired epitope.

In some embodiments, the second portion of the singlet aptamer is hybridized to a first epitope haplomer and/or a second epitope haplomer. When an aptamer is hybridized to a first epitope haplomer or a second epitope haplomer, the complex thus formed is termed herein the “aptamer-haplomer” complex. When an aptamer is hybridized to a first epitope haplomer and a second epitope haplomer, the complex thus formed is termed herein the “aptamer-haplomers” complex. In some embodiments, the second portion of the singlet aptamer (although complementary to both the first and second epitope haplomers) is not hybridized to the first epitope haplomer and/or the second epitope haplomer.

The present disclosure also provides methods for the directed assembly of an epitope on a target cell using a dual proximal aptamer pair, wherein the epitope is recognized and is able to interact or bind to a recognition molecule. In this embodiment, the method comprises contacting a contacting the target cell with a dual proximal aptamer pair. The dual proximal aptamer pair comprises a first aptamer and a second aptamer. The first aptamer comprises: i) a first portion folded into a tertiary structure that is able to bind to a target molecule on the surface of the target cell; and ii) a second portion comprising a nucleic acid molecule linked to the first portion at either the 3′ or 5′ terminal end of the second portion. The second aptamer comprises: i) a first portion folded into a tertiary structure that is able to bind to a target molecule on the surface of the target cell; and ii) a second portion comprising a nucleic acid molecule linked to the first portion at either the 3′ or 5′ terminal end of the second portion. The method further comprises contacting the target cell with a first epitope haplomer and a second epitope haplomer. The first epitope haplomer comprises: i) a nucleic acid molecule that is complementary to the second portion of the first aptamer; and ii) a reactive effector moiety that is a first portion of the epitope. The second epitope haplomer comprises: i) a nucleic acid molecule that is complementary to the second portion of the second aptamer; and ii) a reactive effector moiety that is a second portion of the epitope. The nucleic acid molecule of the first epitope haplomer is complementary to a region of the second portion of the first aptamer that is in spatial proximity to the region of the second portion of the second aptamer to which the nucleic acid molecule of the second epitope haplomer is complementary. The reactive effector moiety of the first epitope haplomer is in spatial proximity to the reactive effector moiety of the second epitope haplomer, thereby resulting in the directed assembly of the epitope.

The dual proximal aptamer pair comprises a first aptamer and a second aptamer. The first aptamer comprises: i) a first portion folded into a tertiary structure that is able to bind to a target molecule on the surface of the target cell; and ii) a second portion comprising a nucleic acid molecule linked to the first portion at either the 3′ or 5′ terminal end of the second portion. In some embodiments, the second portion of the first aptamer is linked to the first portion of the first aptamer at the 3′ terminal end of the second portion. In some embodiments, the second portion of the first aptamer is linked to the first portion of the first aptamer at the 5′ terminal end of the second portion. The second aptamer also comprises: i) a first portion folded into a tertiary structure that is able to bind to a target molecule on the surface of the target cell; and ii) a second portion comprising a nucleic acid molecule linked to the first portion at either the 3′ or 5′ terminal end of the second portion. In some embodiments, the second portion of the second aptamer is linked to the first portion of the second aptamer at the 3′ terminal end of the second portion. In some embodiments, the second portion of the second aptamer is linked to the first portion of the second aptamer at the 5′ terminal end of the second portion. In some embodiments, both the first aptamer and the second aptamer bind to the same target molecule such that the aptamer pair is in physical proximity. In some embodiments, the first aptamer and the second aptamer bind to a different target molecule on the same cell such that the aptamer pair is in physical proximity.

In some embodiments, the first portion of the first aptamer and the first portion of the second aptamer (that are each folded into tertiary structures that are able to bind to a target molecule) are nucleic acid molecules. In some embodiments, the nucleic acid molecule that is the first portion of the first aptamer and the nucleic acid molecule that is the first portion of the second aptamer each, independently, comprises from about 20 nucleotides to about 150 nucleotides, from about 20 nucleotides to about 120 nucleotides, from about 20 nucleotides to about 100 nucleotides, from about 20 nucleotides to about 80 nucleotides, or from about 20 nucleotides to about 60 nucleotides. In some embodiments, the nucleic acid molecule that is the first portion of the first aptamer and the nucleic acid molecule that is the first portion of the second aptamer each, independently, comprises from about 20 nucleotides to about 80 nucleotides. In some embodiments, the nucleic acid molecule that is the first portion of the first aptamer and the nucleic acid molecule that is the first portion of the second aptamer each, independently, comprises from about 25 nucleotides to about 50 nucleotides. In some embodiments, the nucleic acid molecule that is the first portion of the first aptamer and the nucleic acid molecule that is the first portion of the second aptamer each, independently, has a Tm from about 45° to about 65° C. In some embodiments, the nucleic acid molecule that is the first portion of the first aptamer and the nucleic acid molecule that is the first portion of the second aptamer each, independently, has a Tm from about 45° to about 55° C. In some embodiments, the nucleic acid molecule that is the first portion of the first aptamer and the nucleic acid molecule that is the first portion of the second aptamer each, independently, has a Tm from about 55° to about 65° C. In some embodiments, the first portion of the first aptamer and the first portion of the second aptamer each, independently, comprises either the 3′ or 5′ terminal end of the respective aptamer. In some embodiments, the first portion of the first aptamer and the first portion of the second aptamer each, independently, comprises the 3′ terminal end of the respective aptamer. In some embodiments, the first portion of the first aptamer and the first portion of the second aptamer each, independently, comprises the 5′ terminal end of the respective aptamer.

The first and second aptamers also each comprise a second portion that comprises either the 3′ or 5′ terminal end of the aptamer (i.e., whichever terminal end is not a part of the first portion). Thus, in some embodiments, the first portion of each of the first and second aptamers that is folded into a tertiary structure that is able to bind to a target molecule comprises the 5′ portion of the aptamer, leaving the second portion of each of the first and second aptamers to comprise the 3′ terminal end of the aptamer. Alternately, the first portion of each of the first and second aptamers that is folded into a tertiary structure that is able to bind to a target molecule can comprise the 3′ portion of the aptamer, leaving the second portion to comprise the 5′ terminal end of the aptamer. Alternately, the first portion of the first aptamer that is folded into a tertiary structure that is able to bind to a target molecule comprises the 5′ portion of the aptamer, leaving the second portion of the first aptamer to comprise the 3′ terminal end of the aptamer, and the first portion of the second aptamer that is folded into a tertiary structure that is able to bind to a target molecule comprises the 3′ portion of the aptamer, leaving the second portion of the second aptamer to comprise the 5′ terminal end of the aptamer. Alternately, the first portion of the first aptamer that is folded into a tertiary structure that is able to bind to a target molecule comprises the 3′ portion of the aptamer, leaving the second portion of the first aptamer to comprise the 5′ terminal end of the aptamer, and the first portion of the second aptamer that is folded into a tertiary structure that is able to bind to a target molecule comprises the 5′ portion of the aptamer, leaving the second portion of the second aptamer to comprise the 3′ terminal end of the aptamer.

The second portion of the first and second aptamers comprises a nucleic acid molecule. In some embodiments, the second portion of the first and second aptamers each, independently, comprises from about 25 nucleotides to about 100 nucleotides, from about 25 nucleotides to about 90 nucleotides, from about 25 nucleotides to about 80 nucleotides, from about 25 nucleotides to about 70 nucleotides, from about 25 nucleotides to about 60 nucleotides, from about 25 nucleotides to about 50 nucleotides, from about 25 nucleotides to about 40 nucleotides, from about 25 nucleotides to about 30 nucleotides, from about 30 nucleotides to about 100 nucleotides, from about 30 nucleotides to about 90 nucleotides, from about 30 nucleotides to about 80 nucleotides, from about 30 nucleotides to about 70 nucleotides, from about 30 nucleotides to about 60 nucleotides, from about 30 nucleotides to about 50 nucleotides, from about 30 nucleotides to about 40 nucleotides, from about 40 nucleotides to about 100 nucleotides, from about 40 nucleotides to about 90 nucleotides, from about 40 nucleotides to about 80 nucleotides, from about 40 nucleotides to about 70 nucleotides, from about 40 nucleotides to about 60 nucleotides, from about 40 nucleotides to about 50 nucleotides, from about 50 nucleotides to about 100 nucleotides, from about 50 nucleotides to about 90 nucleotides, from about 50 nucleotides to about 80 nucleotides, from about 50 nucleotides to about 70 nucleotides, from about 50 nucleotides to about 60 nucleotides, from about 60 nucleotides to about 100 nucleotides, from about 60 nucleotides to about 90 nucleotides, from about 60 nucleotides to about 80 nucleotides, from about 60 nucleotides to about 70 nucleotides, from about 70 nucleotides to about 100 nucleotides, from about 70 nucleotides to about 90 nucleotides, from about 70 nucleotides to about 80 nucleotides, from about 80 nucleotides to about 100 nucleotides, from about 80 nucleotides to about 90 nucleotides. In some embodiments, the second portion of the first and second aptamers each, independently, comprises from about 25 nucleotides to about 50 nucleotides.

In some embodiments, the first portion and the second portion of each of the first and second aptamers comprise sequence regions that serve as primer binding sites for amplification purposes. In some embodiments, the 5′ terminal region of the first and/or second aptamer contains a first sequence region that serves as a first primer binding site for amplification purposes. In some embodiments, the 3′ terminal region of the first and/or second aptamer contains a second sequence region that serves as a second primer binding site for amplification purposes. Using both amplification primer binding sites in conjunction with the appropriate primers allows for amplification, such as by PCR, of the first and/or second aptamer. In some embodiments, the respective primer binding regions in the second portion of the first and/or second aptamer can also form part of the template regions for producing the functional epitope upon templated assembly.

The first epitope haplomer comprises a nucleic acid molecule that is complementary to the second portion of the first aptamer, and a reactive effector moiety that is a first portion of the epitope. In some embodiments, the 5′ end of the nucleic acid molecule of the first epitope haplomer is conjugated to the reactive effector moiety. In some embodiments where the reactive effector molecule is a peptide (e.g., the peptide is a first portion of the epitope), the 5′ end of the nucleic acid molecule of the first epitope haplomer is conjugated to the N-terminus of the peptide. In some embodiments, the 3′ end of the nucleic acid molecule of the first epitope haplomer is conjugated to the reactive effector moiety. In some embodiments where the reactive effector molecule is a peptide (e.g., the peptide is a first portion of the epitope), the 3′ end of the nucleic acid molecule of the first epitope haplomer is conjugated to the N-terminus of the peptide.

The second epitope haplomer comprises a nucleic acid molecule that is complementary to the second portion of the second aptamer, and a reactive effector moiety that is a second portion of the epitope. In some embodiments, the 3′ end of the nucleic acid molecule of the second epitope haplomer is conjugated to the reactive effector moiety. In some embodiments where the reactive effector molecule is a peptide (e.g., the peptide is a second portion of the epitope), the 3′ end of the nucleic acid molecule of the second epitope haplomer is conjugated to the C-terminus of the peptide. In some embodiments, the 5′ end of the nucleic acid molecule of the second epitope haplomer is conjugated to the reactive effector moiety. In some embodiments where the reactive effector molecule is a peptide (e.g., the peptide is a second portion of the epitope), the 5′ end of the nucleic acid molecule of the second epitope haplomer is conjugated to the C-terminus of the peptide.

In some embodiments, the nucleic acid molecules of the first epitope haplomer and the second epitope haplomer each, independently, comprises from about 10 nucleotides to about 30 nucleotides, from about 10 nucleotides to about 25 nucleotides, from about 10 nucleotides to about 20 nucleotides, from about 10 nucleotides to about 18 nucleotides, or from about 10 nucleotides to about 15 nucleotides. In some embodiments, the nucleic acid molecules of the first epitope haplomer and the second epitope haplomer each, independently, comprises from about 6 nucleotides to about 24 nucleotides, from about 8 nucleotides to about 20 nucleotides, or from about 10 nucleotides to about 18 nucleotides. In some embodiments, the nucleic acid molecules of the first epitope haplomer and the second epitope haplomer each, independently, comprises from about 16 nucleotides to about 25 nucleotides.

The nucleic acid molecule of the first epitope haplomer is complementary to a region of the second portion of the first aptamer. The nucleic acid molecule of the second epitope haplomer is complementary to a region of the second portion of the second aptamer. The spacing of the regions of complementarity between the second portion of the first aptamer and the nucleic acid molecule of the first epitope haplomer and the second portion of the second aptamer and the nucleic acid molecule of the second epitope haplomer (i.e., spatial proximity) results in the reactive effector moiety of the first epitope haplomer being in spatial proximity to the reactive effector moiety of the second epitope haplomer. As a result of the spatial proximity between the two reactive effector moieties, the directed assembly of the epitope is accomplished. The reactive effector moiety of the first epitope haplomer and the reactive effector moiety of the second epitope haplomer are in spatial proximity when a chemical reaction (such as any one of the chemical reactions described herein) can occur between the respective reactive effector moieties such that the two reactive effector moieties are joined to form the desired epitope.

In some embodiments, the second portion of the first aptamer is hybridized to a first epitope haplomer or the second portion of the second aptamer is hybridized to a second epitope haplomer. When an aptamer is hybridized to its respective epitope haplomer, the complex thus formed is termed herein the “aptamer-haplomer” complex. In some embodiments, the second portion of the first aptamer (although complementary to the first epitope haplomer) is not hybridized to the first epitope haplomer. In some embodiments, the second portion of the second aptamer (although complementary to the second epitope haplomer) is not hybridized to the second epitope haplomer.

In some embodiments, the 5′ and 3′ terminal ends of the aptamer pair are ligated together.

The present disclosure also provides methods for the directed assembly of an epitope on a target cell using a binary aptamer, wherein the epitope is recognized and is able to interact or bind to a recognition molecule. In this embodiment, the method comprises contacting a target cell with a binary aptamer. The binary aptamer comprises: i) a first portion folded into a tertiary structure that is able to bind to a target molecule on the surface of the target cell; ii) a second portion folded into a tertiary structure that is able to bind to a target molecule on the surface of the target cell; and iii) a third portion comprising a nucleic acid molecule located between the first and second portion. The method further comprises contacting the target cell with a first epitope haplomer and a second epitope haplomer. The first epitope haplomer comprises: i) a nucleic acid molecule that is complementary to the third portion of the binary aptamer; and ii) a reactive effector moiety that is a first portion of the epitope. The second epitope haplomer comprises: i) a nucleic acid molecule that is complementary to the third portion of the binary aptamer; and ii) a reactive effector moiety that is a second portion of the epitope. The nucleic acid molecule of the first epitope haplomer is complementary to a region of the third portion of the binary aptamer that is in spatial proximity to the region of the third portion of the binary aptamer to which the nucleic acid molecule of the second epitope haplomer is complementary. The reactive effector moiety of the first epitope haplomer is in spatial proximity to the reactive effector moiety of the second epitope haplomer, thereby resulting in the directed assembly of the epitope.

In some embodiments, the first portion and/or the second portion of the binary aptamer that is folded into a tertiary structure that is able to bind to a target molecule is a nucleic acid molecule. In some embodiments, the nucleic acid molecules that are the first portion and second portion of the binary aptamer each, independently, comprises from about 20 nucleotides to about 150 nucleotides, from about 20 nucleotides to about 120 nucleotides, from about 20 nucleotides to about 100 nucleotides, from about 20 nucleotides to about 80 nucleotides, or from about 20 nucleotides to about 60 nucleotides. In some embodiments, the nucleic acid molecules that are the first portion and the second portion of the binary aptamer each, independently, comprises from about 20 nucleotides to about 80 nucleotides. In some embodiments, the nucleic acid molecules that are the first portion and the second portion of the binary aptamer each, independently, comprises from about 40 nucleotides to about 60 nucleotides. In some embodiments, the first portion of the binary aptamer that is folded into a tertiary structure comprises either the 3′ or 5′ terminal end of the binary aptamer. In some embodiments, the first portion of the binary aptamer that is folded into a tertiary structure comprises the 3′ terminal end of the aptamer. In some embodiments, the first portion of the binary aptamer that is folded into a tertiary structure comprises the 5′ terminal end of the aptamer. In some embodiments, the second portion of the binary aptamer that is folded into a tertiary structure comprises either the 3′ or 5′ terminal end of the binary aptamer. In some embodiments, the second portion of the binary aptamer that is folded into a tertiary structure comprises the 3′ terminal end of the aptamer. In some embodiments, the second portion of the binary aptamer that is folded into a tertiary structure comprises the 5′ terminal end of the aptamer.

In some embodiments, the first portion of the binary aptamer and/or the second portion each, independently, has a Tₘ from about 45° to about 85° C., from about 45° to about 80° C., from about 45° to about 75° C., from about 50° to about 70° C., from about 50° to about 65° C., from about 55° to about 70° C., or from about 55° to about 65° C. In some embodiments, the nucleic acid molecules that are the first portion and the second portion of the binary aptamer each, independently, has a Tm from about 55° to about 65° C.

The binary aptamer also comprises a third portion comprising a nucleic acid molecule located between the first and second portion. In some embodiments, the third portion of the binary aptamer comprises from about 30 nucleotides to about 100 nucleotides, from about 30 nucleotides to about 90 nucleotides, from about 30 nucleotides to about 80 nucleotides, from about 30 nucleotides to about 70 nucleotides, from about 30 nucleotides to about 60 nucleotides, from about 30 nucleotides to about 50 nucleotides, from about 30 nucleotides to about 40 nucleotides, from about 40 nucleotides to about 100 nucleotides, from about 40 nucleotides to about 90 nucleotides, from about 40 nucleotides to about 80 nucleotides, from about 40 nucleotides to about 70 nucleotides, from about 40 nucleotides to about 60 nucleotides, from about 40 nucleotides to about 50 nucleotides, from about 50 nucleotides to about 100 nucleotides, from about 50 nucleotides to about 90 nucleotides, from about 50 nucleotides to about 80 nucleotides, from about 50 nucleotides to about 70 nucleotides, from about 50 nucleotides to about 60 nucleotides, from about 60 nucleotides to about 100 nucleotides, from about 60 nucleotides to about 90 nucleotides, from about 60 nucleotides to about 80 nucleotides, from about 60 nucleotides to about 70 nucleotides, from about 70 nucleotides to about 100 nucleotides, from about 70 nucleotides to about 90 nucleotides, from about 70 nucleotides to about 80 nucleotides, from about 80 nucleotides to about 100 nucleotides, from about 80 nucleotides to about 90 nucleotides. In some embodiments, the third portion of the binary aptamer comprises from about 30 nucleotides to about 60 nucleotides.

In some embodiments, the third portion can be fully random (i.e., 25:25:25:25 dA:dC:dG:dT by synthetic ratios), or with any form of specific patterning, where defined bases are interspersed with random regions. As a non-limiting example, a random region of 61 bases designed to enhance selection of G Quadruplexes can take the form of (N₉-G₄)₄-N₉.

In some embodiments, both the first portion and the second portion of the binary aptamer comprise sequence regions that serve as primer binding sites for amplification purposes. In some embodiments, the 5′ terminal region of the binary aptamer contains a first sequence region that serves as a first primer binding site for amplification purposes. In some embodiments, the 3′ terminal region of the binary aptamer contains a second sequence region that serves as a second primer binding site for amplification purposes. Using both amplification primer binding sites in conjunction with the appropriate primers allows for amplification, such as by PCR, of the binary aptamer. In some embodiments, the respective primer binding regions in the third portion of the binary aptamer can also form part of the template regions for producing the functional epitope upon templated assembly.

In some embodiments, the third portion of the binary aptamer also comprises the 3′ primer biding region for application of the first portion (along with the 5′ primer binding region of the first portion) and the 5′ primer binding region for amplification of the second portion (along with the 3′ primer binding region of the second portion).

In some embodiments, the 3′ terminal end of a first aptamer of a dual proximity aptamer pair and the 5′ terminal end of a second aptamer of a dual proximity aptamer pair can be ligated together to form the binary aptamer. For example, referring to the dual proximal nucleic acid aptamer pair, the 5′ and 3′ terminal ends of the aptamer pair can be ligated together.

The first epitope haplomer comprises a nucleic acid molecule that is complementary to the third portion of the binary aptamer, and a reactive effector moiety that is a first portion of the epitope. In some embodiments, the 5′ end of the nucleic acid molecule of the first epitope haplomer is conjugated to the reactive effector moiety. In some embodiments where the reactive effector molecule is a peptide (e.g., the peptide is a first portion of the epitope), the 5′ end of the nucleic acid molecule of the first epitope haplomer is conjugated to the N-terminus of the peptide.

The second epitope haplomer also comprises a nucleic acid molecule that is complementary to the third portion of the binary aptamer, and a reactive effector moiety that is a second portion of the epitope. In some embodiments, the 3′ end of the nucleic acid molecule of the second epitope haplomer is conjugated to the reactive effector moiety. In some embodiments where the reactive effector molecule is a peptide (e.g., the peptide is a second portion of the epitope), the 3′ end of the nucleic acid molecule of the second epitope haplomer is conjugated to the C-terminus of the peptide.

In some embodiments, the nucleic acid molecules of the first epitope haplomer and the second epitope haplomer each, independently, comprises from about 10 nucleotides to about 30 nucleotides, from about 10 nucleotides to about 25 nucleotides, from about 10 nucleotides to about 20 nucleotides, from about 10 nucleotides to about 18 nucleotides, or from about 10 nucleotides to about 15 nucleotides. In some embodiments, the nucleic acid molecules of the first epitope haplomer and the second epitope haplomer each, independently, comprises from about 6 nucleotides to about 24 nucleotides, from about 8 nucleotides to about 20 nucleotides, or from about 10 nucleotides to about 18 nucleotides. In some embodiments, the nucleic acid molecules of the first epitope haplomer and the second epitope haplomer each, independently, comprises from about 16 nucleotides to about 25 nucleotides.

The nucleic acid molecule of the first epitope haplomer is complementary to a region of the third portion of the binary aptamer. The nucleic acid molecule of the second epitope haplomer is also complementary to a region of the third portion of the binary aptamer. The region of the third portion of the binary aptamer to which the nucleic acid molecule of the first epitope haplomer is complementary is 5′ (referring to the third portion of the singlet aptamer) compared to the region of the third portion of the binary aptamer to which the nucleic acid molecule of the second epitope haplomer is complementary. In some embodiments, the region of the third portion of the binary aptamer between which the nucleic acid molecule of the first epitope haplomer is complementary and the nucleic acid molecule of the second epitope haplomer is complementary comprises from about 18 nucleotides to about 100 nucleotides, from about 18 nucleotides to about 90 nucleotides, from about 18 nucleotides to about 80 nucleotides, from about 18 nucleotides to about 70 nucleotides, from about 18 nucleotides to about 60 nucleotides, from about 18 nucleotides to about 50 nucleotides, from about 18 nucleotides to about 40 nucleotides, from about 18 nucleotides to about 30 nucleotides, from about 18 nucleotides to about 25 nucleotides, from about 20 nucleotides to about 100 nucleotides, from about 20 nucleotides to about 90 nucleotides, from about 20 nucleotides to about 80 nucleotides, from about 20 nucleotides to about 70 nucleotides, from about 20 nucleotides to about 60 nucleotides, from about 20 nucleotides to about 50 nucleotides, from about 20 nucleotides to about 40 nucleotides, from about 20 nucleotides to about 30 nucleotides, from about 30 nucleotides to about 100 nucleotides, from about 30 nucleotides to about 90 nucleotides, from about 30 nucleotides to about 80 nucleotides, from about 30 nucleotides to about 70 nucleotides, from about 30 nucleotides to about 60 nucleotides, from about 30 nucleotides to about 50 nucleotides, from about 30 nucleotides to about 40 nucleotides, from about 40 nucleotides to about 100 nucleotides, from about 40 nucleotides to about 90 nucleotides, from about 40 nucleotides to about 80 nucleotides, from about 40 nucleotides to about 70 nucleotides, from about 40 nucleotides to about 60 nucleotides, from about 40 nucleotides to about 50 nucleotides, from about 50 nucleotides to about 100 nucleotides, from about 50 nucleotides to about 90 nucleotides, from about 50 nucleotides to about 80 nucleotides, from about 50 nucleotides to about 70 nucleotides, from about 50 nucleotides to about 60 nucleotides, from about 60 nucleotides to about 100 nucleotides, from about 60 nucleotides to about 90 nucleotides, from about 60 nucleotides to about 80 nucleotides, from about 60 nucleotides to about 70 nucleotides, from about 70 nucleotides to about 100 nucleotides, from about 70 nucleotides to about 90 nucleotides, from about 70 nucleotides to about 80 nucleotides, from about 80 nucleotides to about 100 nucleotides, from about 80 nucleotides to about 90 nucleotides, or from about 90 nucleotides to about 100 nucleotides. In some embodiments, the region of the third portion of the binary aptamer between which the nucleic acid molecule of the first epitope haplomer is complementary and the nucleic acid molecule of the second epitope haplomer is complementary comprises from about 18 nucleotides to about 25 nucleotides.

The spacing of the regions of complementarity between the third portion of the binary aptamer and the nucleic acid molecules of the first and second epitope haplomers (i.e., spatial proximity) results in the reactive effector moiety of the first epitope haplomer being in spatial proximity to the reactive effector moiety of the second epitope haplomer. As a result of the spatial proximity between the two reactive effector moieties, the directed assembly of the epitope is accomplished. The reactive effector moiety of the first epitope haplomer and the reactive effector moiety of the second epitope haplomer are in spatial proximity when a chemical reaction (such as any one of the chemical reactions described herein) can occur between the respective reactive effector moieties such that the two reactive effector moieties are joined to form the desired epitope.

In some embodiments, the third portion of the binary aptamer is hybridized to a first epitope haplomer and/or a second epitope haplomer. When an aptamer is hybridized to a first epitope haplomer or a second epitope haplomer, the complex thus formed is termed herein the “aptamer-haplomer” complex. When an aptamer is hybridized to a first epitope haplomer and a second epitope haplomer, the complex thus formed is termed herein the “aptamer-haplomers” complex. In some embodiments, the third portion of the binary aptamer (although complementary to both the first and second epitope haplomers) is not hybridized to the first epitope haplomer and/or the second epitope haplomer.

In principle, a pair of molecules (i.e., partial effector moieties as previously described in, for example, PCT International Publication WO 14/197547; now referred to herein as “haplomers”) covalently carrying reactive effector moieties (i.e., combinable portions of a desired effector product such as an epitope for a recognition molecule) can complete effector product assembly upon any templating structure, provided that the template-ligand (i.e., aptamer-haplomer) binding results in the spatial proximity for mutual reactivity between two reactive effector moieties to occur. Accordingly, other molecules beyond nucleic acids can, in principle, act as guides for specific templated assembly processes. Such non-nucleic acid templates may include proteins and complex carbohydrates, either alone or in combination. Also, either proteins or complex carbohydrates can, in principle, act as templates in concert with nucleic acids, where each are present within specific ribonucleoproteins, with or without glycosyl modifications.

An approach where few assumptions are made as to the nature of analog templating sites uses nucleic acid aptamers. Here aptamers are selected as ligands themselves for proteomic/glycomic/nucleic acid targets, and those binding to targets in spatial proximity are potentially useful as carriers of haplomers for templated assembly. Pairs of aptamers can be used as such carrier ligands, or alternatively a single selected aptamer can be used in concert with a known ligand, also carrying a haplomer.

Since aptamers can be selected to bind to non-nucleic acid target molecules expressed on cell surfaces, they are particularly useful for recognition and adaptive templating of novel surface structures found on specific cells, such as tumor cells. However, since most aptamers are not large nucleic acid molecules (i.e., many are less than 100 bases), and may often assume a folded and compacted structure, they are more readily transfected into target cells than many protein-based reagents. Thus, intracellular targets for aptamers are also desired. Such intracellular targets can also include RNA molecules, particularly where the RNA exists in a well-folded stable state. The latter configurations may often be refractory to conventional hybridization-mediated templated assembly, but amenable to recognition and secondary adaptive templating by aptamers.

Aptamers can be single-stranded folded nucleic acid molecules which have been selected for the ability to bind to a specific target molecule of interest. In some embodiments, the selection process involves the synthesis of a nucleic acid molecule with an extended random tract flanked on the 3′ or 5′ terminal end by specific primer sequences which enable amplification of the random population, or any members thereof with specific sequences. Within a large random population, a library of structural motifs arising from self-folding of the random region is generated and, in principle, a wide range of target molecules can be bound by specific members of this library. These specific binding nucleic acid molecules can be enriched by appropriate selection procedures, and then amplified. After such amplification of the initially very small subset of nucleic acid molecules that bind a desired target molecule, the selection round is repeated, promoting further enrichment of the desired nucleic acid molecules. In addition, this cycle is evolutionary, since mutations arising during the amplification process which enhance binding are favored and, after sufficient repetitions, specific nucleic acid molecules which bind with high affinity to the desired target molecule of interest can be isolated and identified. Such specific nucleic acid molecules that bind to the desired target molecule of interest with high affinity serve as nucleic acid aptamers, which in turn can serve as templates for templated assembly of functional products that can modify a cell.

In general, since aptamers can be composed of nucleotides, they can potentially provide a short linear sequence for templating purposes, as a contiguous segment of their primary sequences. Such a “built-in” templating sequence can, in principle, be located anywhere within the primary aptamer sequence, provided that hybridization of haplomers onto the aptamer does not disrupt binding of the aptamer to the target molecule of interest. In practice, though, targeting of either 5′ or 3′ terminal regions of aptamer sequences is likely to have a lower probability of disrupting aptamer function. Such terminal sites are easier to modify as desired, or to generate as secondarily appended segments.

In any of the target molecule binding components, aptamers, or epitope haplomers described herein, the nucleic acid molecules that form one or more portions thereof can comprise DNA nucleotides, RNA nucleotides, phosphorothioate-modified nucleotides, 2-O-alkylated RNA nucleotides, halogenated nucleotides, locked nucleic acid nucleotides (LNA), peptide nucleic acids (PNA), XNA, morpholino nucleic acid analogues (morpholinos), pseudouridine nucleotides, xanthine nucleotides, hypoxanthine nucleotides, 2′-deoxyinosine nucleotides, or other nucleic acid analogues capable of base-pair formation, or any combination thereof. In some embodiments, the nucleic acid is or comprises a portion which is LNA. In some embodiments, the hybridization region of the epitope haplomer and the portion of the aptamer which hybridizes to the hybridization region of the epitope haplomer both comprise L-DNA. For example, the nucleic acid molecule of either or both of the first epitope haplomer and the second epitope haplomer, and the portion of the aptamer which is complementary thereto to, both comprise L-DNA. In addition, aptamers can be very flexible. For example, aptamers can be modified such that nuclease resistance is conferred by means of modified backbones, including, but not limited to, phosphorothioates, or 2′ modifications, including, but not limited to, 2′-O-methyl derivatives. Alternately, L-DNA analogs (spiegelmers) binding desired targets can be used where applicable, and have high nuclease resistance.

In some embodiments, the C-terminus of the reactive effector moiety (e.g., polypeptide; first portion of the epitope) of the first epitope haplomer further comprises a first bio-orthogonal reactive group and the N-terminus of the reactive effector moiety (e.g., polypeptide; second portion of the epitope) of the second epitope haplomer further comprises a second bio-orthogonal reactive group, wherein the first bio-orthogonal reactive group and the second bio-orthogonal reactive group are compatible. In some embodiments, the C-terminus of the reactive effector moiety (e.g., polypeptide; first portion of the epitope) of the first epitope haplomer does not further comprise a first bio-orthogonal reactive group and the N-terminus of the reactive effector moiety (e.g., polypeptide; second portion of the epitope) of the second epitope haplomer does not further comprise a second bio-orthogonal reactive group (i.e., where covalent joining of the two portions of the epitope is not desired).

In some embodiments, the first bio-orthogonal reactive group is a linear alkyne and the second bio-orthogonal reactive group is an azide, or the second bio-orthogonal reactive group is a linear alkyne and the first bio-orthogonal reactive group is an azide. In some embodiments, the first bio-orthogonal reactive group is a strained alkyne and the second bio-orthogonal reactive group is an azide or the second bio-orthogonal reactive group is a strained alkyne and the first bio-orthogonal reactive group is an azide. In some embodiments, the first bio-orthogonal reactive group is a tetrazine and the second bio-orthogonal reactive group is a cyclooctene or the second bio-orthogonal reactive group is a tetrazine and the first bio-orthogonal reactive group is a cyclooctene.

In some embodiments, the C-terminus of the reactive effector moiety (e.g., polypeptide; first portion of the epitope) of the first epitope haplomer further comprises a first chemical modification and the N-terminus of the reactive effector moiety (e.g., polypeptide; second portion of the epitope) of the second epitope haplomer further comprises a second chemical modification, wherein the first chemical modification and the second chemical modification are compatible. In some embodiments, the first chemical modification is amidation (CONH₂) or esterification (COOR), where R is methyl, ethyl, or phenyl, and the second chemical modification is acetylation or an N-methyl substitution of the N-terminal amino group. Additional bio-orthogonal reactive groups and chemical modifications are set forth below.

In some embodiments, covalent joining of the two epitope segments via the respective reactive effector moieties of the first and second epitope haplomers may not be necessary, where the combined binding affinity of the two half or split epitopes within the binding site of the recognition molecule of interest reaches a significant fraction of the affinity towards the original epitope. This has the opportunity to occur where thermal motions of the two epitope subsegments are constrained by their enforced (template-mediated) spatial proximity. The effective affinity enhancement is analogous to the avidity benefit conferred from binding of a bivalent recognition molecule towards a target with two or more linked epitopes.

In order for two epitope fragments to fit within a recognition molecule binding site in the absence of covalent joining, in some embodiments chemical modifications may be desired at the C-terminal and N-terminal ends of the N-terminal and C-terminal subsegments, respectively. This may arise from the introduction of new —COOH and NH₂-groups as a consequence of splitting of a contiguous peptide sequence, where these moieties may be poorly compatible with the local chemical environment within the recognition molecule binding site.

Chemical modifications for the C-terminal end of the N-terminal epitope fragment include, but are not limited to, amidation (CONH₂) and esterification (COOR), where R may be, but is not limited to, methyl, ethyl, or phenyl groups. Chemical modifications for the N-terminal end of the C-terminal epitope fragment include, but are not limited to, acetylation or N-methyl substitutions of the N-terminal amino group.

In some embodiments, the terminal end of the first epitope haplomer (comprising the reactive effector moiety) that is in spatial proximity with the terminal end of the second epitope haplomer (comprising the reactive effector moiety) are covalently joined. In some embodiments, the first epitope haplomer and the second epitope haplomer are covalently joined to their respective ends in spatial proximity by a chemical reaction occurring between their respective reactive effector moieties. Numerous reactive effector moieties are disclosed in, for example, PCT International Publication WO 14/197547.

The combination of two reactive effector moieties allows the formation of a functional product (e.g., an epitope for a recognition molecule). The interaction between two reactive effector moieties can include physical interactions, such as chemical bonds (either directly linked or through intermediate structures), as well as non-physical interactions and attractive forces, such as electrostatic attraction, hydrogen bonding, and van der Waals/dispersion forces.

A reactive effector moiety can be biologically inert. In particular, the reactive effector moiety associated with a first epitope haplomer can interact with a corresponding reactive effector moiety associated with a second epitope haplomer, but will not readily interact with natural biomolecules. This is to ensure that the templated assembly product is formed only when corresponding effector partial moieties are assembled on an aptamer(s) bound to a target molecule. It also safeguards the reactive effector moiety from reacting with functional groups on other molecules present in the environment in which the assembly occurs, thus preventing the formation of unintended products. An example of a reactive effector moiety includes a bio-orthogonal moiety. A bio-orthogonal moiety reacts chemically with a corresponding bio-orthogonal moiety and does not readily react chemically with other biomolecules.

The reactive effector moiety provides a mechanism for templated reactions to occur in complex target compartments, such as a cell, virus, tissue, tumor, lysate, other biological structure, or spatial region within a sample that contains the target molecule, or that contains a different amount of target molecule than a non-target compartment. A reactive effector moiety can react with a corresponding reactive effector moiety, but does not react with common biochemical molecules under typical conditions. Unlike other reactive entities, the selectivity of reactive effector moiety prevents ablation of the reactive group prior to assembly of the product or reactant.

An example of a reactive effector moiety can include a bio-orthogonal moiety. The bio-orthogonal moiety can include those groups that can undergo “click” reactions between, for example, azides and alkynes, traceless or non-traceless Staudinger reactions between azides and phosphines, and native chemical ligation reactions between thioesters and thiols. Additionally, the bio-orthogonal moiety can be any of an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, a quadricyclane, and derivatives thereof. Multiple reactive effector moieties can be used with the methods and compositions disclosed herein. Some non-limiting examples include the following.

Click chemistry is highly selective as neither azides nor alkynes react with common biomolecules under typical conditions. Azides of the form R—N₃ and terminal alkynes of the form R—C≡CH or internal alkynes of the form R—C≡C—R react readily with each other to produce Huisgen cycloaddition products in the form of 1,2,3-triazoles. Azides and azide derivatives may be readily prepared from commercially available reagents. Azides can also be introduced to a reactive effector moiety during synthesis of the reactive effector moiety. In some embodiments, an azide group is introduced into an reactive effector moiety comprised of a peptide by incorporation of a commercially available azide-derivatized standard amino acid or amino acid analogue during synthesis of the reactive effector moiety peptide using standard peptide synthesis methods. Amino acids may be derivatized with an azide replacing the α-amino group. Commercially available products can introduce azide functionality as an amino acid side chain, resulting in a structure of the form:

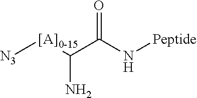

where A is any atom and its substituents in a side chain of a standard amino acid or non-standard amino acid analogue.

An azide may also be introduced into an reactive effector moiety peptide after synthesis by conversion of an amine group on the peptide to an azide by diazotransfer methods. Bioconjugate chemistry can also be used to join commercially available derivatized azides to chemical linkers, or reactive effector moieties that contain suitable reactive groups.

Standard alkynes can also be incorporated into reactive effector moieties by methods similar to azide incorporation. Alkyne-functionalized nucleotide analogues are commercially available, allowing alkyne groups to be directly incorporated at the time of reactive effector moiety synthesis. Similarly, alkyne-derivatized amino acid analogues may be incorporated into an reactive effector moiety by standard peptide synthesis methods. Additionally, diverse functionalized alkynes compatible with bioconjugate chemistry approaches may be used to facilitate the incorporation of alkynes to other moieties through suitable functional or side groups.

Standard azide-alkyne chemistry reactions typically require a catalyst, such as copper(I). Since copper(I) at catalytic concentrations is toxic to many biological systems, standard azide-alkyne chemistry reactions have limited uses in living cells. Copper-free click chemistry systems based on activated alkynes circumvent toxic catalysts. Activated alkynes often take the form of cyclooctynes, where incorporation into the cyclooctyl group introduces ring strain to the alkyne.

Heteroatoms or substituents may be introduced at various locations in the cyclooctyl ring, which may alter the reactivity of the alkyne or afford other alternative chemical properties in the compound. Various locations on the ring may also serve as attachment points for linking the cyclooctyne to a reactive effector moiety or linker. These locations on the ring or its substituents may optionally be further derivatized with accessory groups. Multiple cyclooctynes are commercially available, including several derivatized versions suitable for use with standard bioconjugation chemistry protocols. Commercially available cyclooctyne derivatized nucleotides can aid in facilitating convenient incorporation of the reactive effector moiety during nucleic acid synthesis.

The Staudinger reduction, based on the rapid reaction between an azide and a phosphine or phosphite with loss of N₂, also represents a bio-orthogonal reaction. The Staudinger ligation, in which covalent links are formed between the reactants in a Staudinger reaction, is suited for use in templated assembly. Both non-traceless and traceless forms of the Staudinger ligation allow for a diversity of options in the chemical structure of products formed in these reactions.

The standard Staudinger ligation is a non-traceless reaction between an azide and a phenyl-substituted phosphine such as triphenylphosphine, where an electrophilic trap substituent on the phosphine, such as a methyl ester, rearranges with the aza-ylide intermediate of the reaction to produce a ligation product linked by a phosphine oxide. Phenyl-substituted phosphines carrying electrophilic traps can also be readily synthesized. Derivatized versions are available commercially and suitable for incorporation into templated assembly reactants.

In some embodiments, phosphines capable of traceless Staudinger ligations can be utilized as reactive effector moieties. In a traceless reaction, the phosphine serves as a leaving group during rearrangement of the aza-ylide intermediate, creating a ligation typically in the form of a native amide bond. Compounds capable of traceless Staudinger ligation generally take the form of a thioester derivatized phosphine or an ester derivatized phosphine. Ester derivatized phosphines can also be used for traceless Staudinger ligation. Thioester derivatized phosphines can also be used for traceless Staudinger ligations.

Chemical linkers or accessory groups can optionally be appended as substituents providing attachment points for reactive effector moieties or for the introduction of additional functionality to the reactant.

Compared to the non-traceless Staudinger phenylphosphine compounds, the orientation of the electrophilic trap ester on a traceless phosphinophenol is reversed relative to the phenyl group. This enables traceless Staudinger ligations to occur in reactions with azides, generating a native amide bond in the product without inclusion of the phosphine oxide. The traceless Staudinger ligation may be performed in aqueous media without organic co-solvents if suitable hydrophilic groups, such as tertiary amines, are appended to the phenylphosphine. Preparation of water-soluble phosphinophenol, which can be loaded with a desired reactive effector moiety containing a carboxylic acid (such as the C-terminus of a peptide) via the mild Steglich esterification using a carbodiimide such as dicyclohexylcarbodiimide (DCC) or N,N′-diisopropylcarbodiimide (DIC) and an ester-activating agent such as 1-hydroxybenzotriazole (HOBT) has been reported (Weisbrod et al., Synlett, 2010, 5, 787-789).

Phosphinomethanethiols represent an alternative to phosphinophenols for mediating traceless Staudinger ligation reactions. In general, phosphinomethanethiols possess favorable reaction kinetics compared with phosphinophenols in mediating traceless Staudinger reaction. U.S. Patent Publication 2010/0048866 and Tam et al., J. Am. Chem. Soc., 2007, 129, 11421-30 describe preparation of water-soluble phosphinomethanethiols. These compounds can be loaded with a peptide or other payload, in the form of an activated ester, to form a thioester suitable for use as a traceless bio-orthogonal reactive group.

Native chemical ligation is a bio-orthogonal approach based on the reaction between a thioester and a compound bearing a thiol and an amine. The classic native chemical ligation is between a peptide bearing a C-terminal thioester and another bearing an N-terminal cysteine. Native chemical ligation can be utilized to mediate traceless reactions producing a peptide or peptidomimetic containing an internal cysteine residue, or other thiol-containing residue if non-standard amino acids are utilized.

N-terminal cysteines can be incorporated by standard amino acid synthesis methods. Terminal thioesters can be generated by several methods known in the art, including condensation of activated esters with thiols using agents such as dicyclohexylcarbodiimide (DCC), or introduction during peptide synthesis via the use of “Safety-Catch” support resins.

Any suitable bio-orthogonal reaction chemistry can be utilized for synthesis of reactive effector moieties, as long as it efficiently mediates a reaction in a highly selective manner in complex biologic environments. A recently developed non-limiting example of an alternative bio-orthogonal chemistry that may be suitable is reaction between tetrazine and various alkenes such as norbornene and trans-cyclooctene, which efficiently mediates bio-orthogonal reactions in aqueous media.

Chemical linkers or accessory groups can optionally be appended as substituents to the above reactants, providing attachment points for nucleic acid moieties or for the introduction of additional functionality to the reactant.

In some embodiments, the first portion of the target molecule binding component is a ligand for the target molecule. In some embodiments, the ligand is alpha-melanocyte stimulating hormone. Additional ligand/target molecules are well known to the skilled artisan.

In some embodiments, the first portion of the target molecule binding component is an aptamer for the target molecule. Aptamers (or the first portion thereof which is able to bind to a desired target molecule) can be selected from a library comprising, for example: binding members of the library to a desired solid phase target; washing the solid phase target; eluting the bound members of the library; precipitating the bound members of the library; reconstituting the bound members of the library; analyzing the bound members of the library for a suitable amplifiable concentration; performing preparative asymmetric PCR; testing the PCR products on a gel; binding the PCR products to streptavidin magnetic beads; washing the streptavidin magnetic beads; eluting the top strands; testing the eluted strands on a gel; and performing the cycle a plurality of times, such as up to nine or more times, until diversity of the binding aptamer population is sufficiently reduced such that analysis of the binding properties of specific predominant aptamer clones can be performed.

A general binding and elution procedure is described herein. In some embodiments, aptamers are initially prepared in standard phosphate-buffered saline with 1 mM magnesium chloride (PBSM), and heated for about 3 minutes at 80° C., followed by at least 5 minutes at 0° C. (ice bath) to allow for self-annealing and to minimize inter-aptamer interactions. In some embodiments, aptamer populations (or specific aptamers) are incubated with a target and rendered solid-phase.

In some embodiments, aptamer populations (or specific aptamers) can be incubated with a target for at least 1 hour at room temperature, then added to an excess of solid-phase capture matrix for greater than about 1 hour at room temperature. For primary aptamer populations, the initial incubation time with the target in solution is about 16 hours. For successive rounds of selected aptamer populations, the incubation time is about 2 to 4 hours. For specific aptamers, the incubation time is about 1 hour. Where the target is biotinylated, the capture matrix can be excess streptavidin magnetic beads (SAMBs) or any other streptavidin resin. Bead quantities can be calculated from the known molar input of biotinylated target and the maximal bead binding capacity data as provided by the manufacturer. SAMBs can be initially prepared by taking a predetermined volume in a storage buffer based on experimental needs, magnetically separating them, and washing twice with, for example, 1.0 ml of PBSM, using magnetic separation each time. Finally, the beads can be resuspended in the original volume of PBSM.

In other embodiments, the aptamer population (or specific aptamers) can be incubated with a target that has been previously rendered solid-phase on a suitable matrix. These matrices include, but are not limited to, streptavidin magnetic beads, streptavidin agarose, or any other streptavidin resin, where the target bears one or more biotin moieties. The target can also be covalently bound to the solid-phase matrix through various chemistries including, but not limited to, amine/N-hydroxy-succinimide, or thiol/maleimide. Such chemistries can covalently bind targets to magnetic beads or various other materials including, but not limited to, agarose or a polymerized resin.

In some embodiments, aptamers captured on solid-phase target matrix can be washed. The solid-phase matrices bearing targets and bound aptamers can be washed 1 time, 2 times, 3 times, or 4 times with, for example, 0.5 ml PBSM, with a final resuspension in the same volume of PBSM. Where SAMBs provide the solid-phase matrix, separations of matrix from supernatants during each wash cycle can be carried out by means of magnetic separation. Where other solid-phase materials are used, separations can be carried out by other means including, but not limited to, centrifugation or filtration.

In some embodiments, the bound aptamers are eluted. Aptamer/solid-phase target matrices can be separated from the final wash supernatants, as in the wash step above, and resuspended in, for example, about 100 μl of 0.1 M sodium hydroxide/5 mM EDTA for about 20 seconds at room temperature. The supernatant can be removed to a fresh tube, and the solid-phase material resuspended in, for example, about 100 μl of 0.1 M sodium hydroxide/5 mM EDTA for about 20 seconds at room temperature once more. Both supernatants can be pooled, and precipitated with, for example, about 20 μg of glycogen/20 μl of 3 M sodium acetate/600 μl ethanol for about 30 minutes at about −20° C. Preparations can be centrifuged (e.g., 10 minutes at maximum microfuge speed), and the pellets washed with, for example, 1 ml of 70% ethanol.

In some embodiments, the eluted aptamers can be reconstituted. For example, following the 70% wash from the step above, the preparations can be briefly centrifuged (e.g., for 1-2 minutes at maximum microfuge speed) and the supernatants removed. The resultant pellets can be dried and re-dissolved in an appropriate volume (e.g., usually 25 μl) of TE (10/1.0). Where the separation procedure uses magnetic beads, the resolubilized aptamer preparations can be subjected to another magnetic separation (e.g., 1 minute) to remove residual carry-over beads. Aptamer preparations can be quantitated spectrophotometrically at 260 nm, where an absorbance of 1.0=33 μg/ml single-stranded DNA. Samples can also be analyzed on, for example, 10% denaturing urea acrylamide gels. These preparations are termed herein primary eluted single-stranded aptamers for cycle N, where N is the number of times the cycling procedure has been repeated.

In some embodiments, the aptamers can be analyzed after about 9 to 10 cycles. For example, as the binding and elution cycles are continued, the proportion of the aptamer population that significantly binds to the target increases and, likewise, the diversity of the population (corresponding to variation in the N region of the first portion of a singlet aptamer, for example), commencing at maximal (i.e., random) diversity in the initial population) decreases. After about 9 to 10 cycles, typically clonal analysis of the aptamer population demonstrates recurrent independent clones with identical or related sequences, which correspond to population members with significant binding properties.

In some embodiments, the clonal analysis procedure can be carried out as described herein. In general, aptamers can be analyzed by cloning and sequencing at any point during the cycling steps, but typically about 9 to 10 cycles are suitable before multiple recurrent clones with high levels of sequence similarity are obtained. After a desired number of cycles, primary eluted Left or Right aptamers can be amplified with appropriate L/R primers to provide a source of duplexes for cloning. Resulting PCR products can be purified to remove excess primers (NucleoSpin kits, Machery-Nagel/Clontech), and then ligated to a vector suitable for direct cloning of fragments produced by Taq DNA polymerase (including, but not limited to, vectors such as pGEM-Teasy, Promega). After an appropriate ligation incubation, competent E. coli cells can be transformed with the products. Mini-preparations of resulting colonies can then be sequenced with primers spanning the aptamer inserts, and analyzed for clones with similar 40-mer tracts.

For instance, the target molecule can be rendered solid-phase after binding to an aptamer population. In some embodiments, the target molecule can be rendered solid-phase by conjugation to N-hydroxylsuccinimide activated magnetic beads. In some embodiments, the target molecules bear one or more biotin moieties, and can be rendered solid-phase by binding to solid-phase streptavidin matrices (such as streptavidin agarose or streptavidin magnetic beads). The non-binding aptamer species can be removed by washing. Bound aptamers can be eluted with 0.05 or 0.1 M sodium hydroxide, precipitated, washed, and then used for re-amplification to obtain enriched single-stranded DNAs for a subsequent round of selection.

Preparation of single-stranded DNA of the correct strand sense from amplified aptamers can allow for the re-iteration of a subsequent round of selection. A preliminary trial amplification can be used to gauge the best concentrations of eluted aptamers to use in bulk PCR preparations to obtain sizable amounts of single-stranded aptamers. In a typical trial amplification (i.e., “range test”), the primary eluted aptamers from each cycle can be diluted at, for example, 1:100, 1:500, and 1:2000, and 1.0 μl of each used in a PCR amplification with Amplitaq Gold (Thermo) with a cycle of 7 minutes at 95° C., 20×(60° C. for about 20 seconds, 72° C. for about 1 minute, and 94° C. for about 40 seconds), 60° C. for about 20 seconds, and 72° C. for about 2 minutes. Products can be analyzed on, for example, a 10% non-denaturing acrylamide gel to determine the concentrations providing the best and purest product yields free from higher-molecular weight forms arising when the starting target concentration is too high. With this information, single strands can be prepared by several different options, including, but not limited to, electrophoresis, denaturation with biotinylated bottom strand, and asymmetric PCR.

In some embodiments, differential strand biotinylation can be used to prepare large amounts of single-stranded aptamer selected subpopulations. Single-stranded aptamer preparations eluted from a solid-phase target can be amplified where the bottom strand (corresponding to the aptamer complement) bears a 5′-biotin. After binding to solid-phase streptavidin, single-stranded aptamers (top strand) can be eluted with alkali (such as, for example, 0.05M or 0.1M NaOH, also with 5 mM EDTA).

In some embodiments, an asymmetric PCR process can be used for generating single-strands from amplified duplex aptamer populations. A large molar excess of top-strand primer can be used, resulting in generation of an excess of single strands corresponding to the desired aptamer subpopulation. Any biotinylated strands can be removed by, for example, binding to solid-phase streptavidin, with the unbound supernatants containing the appropriate single-stranded preparation. Preparative asymmetric PCR involves an initial amplification of the selected aptamer population where the bottom strand is biotinylated, followed by asymmetric PCR for differential amplification of the top strands. Remaining bottom strands can be removed by binding to SAMBs (as described above, for example).

Following selection for binding, singlet aptamers bound to target may not necessarily provide accessible terminal sequences for hybridization, as these may have become incorporated into the folded structures of specific aptamers in the bound state. Singlets with accessible termini can be selected with an additional step, where the singlet aptamers are bound to non-biotinylated targets, and subsequently hybridized with a biotinylated probe complementary to the desired accessible 3′ or 5′ terminus. Since hybridization requires accessibility, appropriate binders can then be selected on a solid-phase streptavidin matrix such as, but not limited to, streptavidin-magnetic beads. Upon elution, singlet aptamers can be amplified and the process repeated if necessary.

In some embodiments, the preparative asymmetric PCR comprises: amplifying the selected aptamer population where the bottom strand corresponding to the aptamer complement is biotinylated, and performing asymmetric PCR for differential amplification of the top strands, whereby a large molar excess of the top-strand primer is used, resulting in generation of an excess of single strands corresponding to the desired aptamer subpopulation.

In some embodiments, biotinylated strands are removed by binding to solid-phase streptavidin, with the unbound supernatants containing the appropriate single-stranded preparation.

Methods of selecting an aptamer having an accessible 3′ or 5′ terminal end for hybridization to an epitope haplomer can comprise: contacting an aptamer with a corresponding target molecule; contacting the aptamer with a biotinylated probe having a region that is complementary to the 3′ or 5′ terminal end of the aptamer; washing the aptamer-probe complex to remove unbound probe; contacting the aptamer-probe complex with streptavidin magnetic beads; and washing the streptavidin magnetic beads and eluting the aptamer, wherein the aptamer possesses an accessible 3′ or 5′ terminal end for hybridization to an epitope haplomer. This method is shown as one way to select for singlet aptamers presenting accessible sequences after target binding, such that they can be used for subsequent effector partial assembly.

Methods of preparing a binary aptamer can comprising: contacting a target molecule or target cell with a plurality of aptamers; eluting the bound aptamers; contacting the target molecule or target cell with the population of bound aptamers; contacting the bound aptamers with a ligase and an RNA splint; and removing the splint with RNase H, thereby resulting in a covalently ligated binary aptamer.

A general binary aptamer selection process is described herein. For example, left- and right-primary aptamer populations initially selected separately on a specific target can be co-incubated with the target in equimolar quantities. In a typical procedure, 8 pmol of each of L- and R-aptamers and specific target can be used. After about 2 to 4 hour incubation at room temperature, the target can be bound to a solid-phase matrix as described above (i.e., general binding and elution procedure), and subjected to 4×0.5 ml washes with, for example, PBSM. The solid-phase preparation can be annealed with an excess of splint oligonucleotide spanning the 3′ and 5′ ends of the L- and R-aptamers, respectively. Annealing can be carried out with, for example, incubations of about 5 minutes at about 37° C., and about 30 minutes at about 25° C. Preparations can be washed twice with, for example, ×1 ligase buffer with 1 mM ATP (New England Biolabs), and resuspended in about 50 μl of the same ligase buffer. Ligase can be added, and the preparations incubated for about 1.5 to about 4 hours at room temperature. Controls can be used where the splint and ligase, or both, are omitted.

In some embodiments, the ligase is T4 DNA ligase, T3 DNA ligase or Chlorella DNA ligase (SplintR® ligase; New England Biolabs, with corresponding buffers).

In some embodiments, the ligase is T4 DNA ligase or Chlorella DNA ligase.

In some embodiments, the aptamers can be selected to bind to a cancer cell, and wherein aptamers that bind to normal cells can be subtracted.

Ligation of singlet aptamers co-binding on a common target molecule in spatial proximity results in a continuous fusion between Left and Right aptamers, termed a binary aptamer. From any specific binary aptamer or population of binary aptamers, the entire binary sequence can be amplified with a single pair of primers spanning the joined sequence. If desired, from any specific binary aptamer or population of binary aptamers, component Left and Right aptamers can also be amplified.

Binary aptamers offer the advantages of enhanced specificity and affinity, and afford a templating sequence in the interface between the L- and R-aptamer segments. This sequence has a dual role both for templating desired assembly reactions, and also as primer sites for the L-aptamer reverse primer, and R-aptamer forward primer.

Generally, binary aptamers conform to the general pattern: (L-forward primer)-(L-random region)-(L-reverse primer/half-splint region)-(R-forward primer/half-splint region)-(R-random region)-(R-reverse primer). The joined (L-reverse primer/half-splint)-(R-forward primer/half-splint) segment constitutes the site whereby a splint molecule enables L- and R-ligation, and also serves as single-stranded accessible template for template reactions.

The joining of each Left and Right aptamer into a binary form can be effected by, for example, means of an RNA splint oligonucleotide complementary to the 3′ end of the Left aptamer and 5′ end of the Right aptamer. The ligation of the aptamer ends upon this splint can be effected by T4 DNA ligase, or more efficiently by Chlorella DNA ligase (New England Biolabs), which is highly effective in the ligation of DNA ends by RNA splints. Specific binary pairs can be identified and characterized by amplifying the proximal binary units as a single contiguous sequence. After ligation is complete, the RNA splint can be removed by treatment with RNAse H (which is active only on RNA:DNA hybrids), to expose the joined template region from the Left and Right aptamers for subsequent hybridization with haplomers.

Selection of binary aptamers by target co-binding and splint ligation also simultaneously ensures that the template region is accessible for hybridization purposes. Pairs of aptamers in spatial proximity whose 3′ and 5′ ends are inaccessible (as a consequence of their specific target binding) will fail to hybridize with the splint and allow subsequent ligation and amplification as binary entities.

Alternate aptamer selection processes are also disclosed herein. For example, Left and Right aptamer libraries can be initially selected separately on a desired target molecule, and the binding subpopulations eluted. These can then be subjected to co-binding selection for enrichment in proximal binary aptamers, and from the eluted binary populations component Left- and Right-aptamer populations can be amplified. Both of these selected populations can be subjected to recombinatorial DNA shuffling (Stemmer, Nature, 1994, 370, 389-391) to enhance molecular diversity.

The DNA shuffling step (see, molecular breeding, Stemmer 1994) is designed to promote cross-over priming between different aptamer strands, and is effected by limited DNase I digestion of each selected Left and Right aptamer subpopulations, followed by a reassembly cycle, and then re-amplification with the original primers. Products of aptamer DNA shuffling can be selected once more on solid-phase target at high stringency, followed in turn by co-binding ligation, elution, and amplification. Products of this process can be characterized by sequencing and tests for binding affinity.

The manipulations of both singlet and binary aptamers for templated assembly purposes are described as above. Binary aptamer applications can be divided into two categories. In the first category, following their identification from proximally-binding singlets, binaries can be ligated together in solution (in absence of a target molecule) and then deployed for functional purposes. In the second category, binaries are assembled directly on the target molecule, whether through convenience or necessity.

All aptamers generated for adaptive templating purposes can have their binding affinities measured (as indicated by their K_{d} value). Such affinity measurements can be conducted by various methods, including, but not limited to, BiaCore instrumentation, equilibrium dialysis, gel shift assays, filter-binding assays, and quantitative PCR combined with a separation process for bound and unbound material (see, Jing et al., Anal. Chim Acta, 2011, 686, 9-18).

During any application of templated assembly of haplomers, the haplomer hybridization to a desired template can be specific. Non-specific hybridization can be minimized by selecting target molecules that are unique to the cell type of interest. In cases where only a point mutation distinguishes the target molecule, the risk of off-target molecule hybridization is significant. The use of aptamers to provide templates for haplomers provides a unique opportunity to completely eliminate non-target molecule hybridization.

DNA analogs with L-ribose (L-DNA) instead of D-ribose require homochiral complementary nucleic acid strands for duplexes to form. Thus, a template composed of L-DNA cannot hybridize with any natural nucleic acids, which all possess D-ribose. L-DNA template tags can be appended to aptamers, for the purpose of templating effector partial moieties whose hybridization portions also are comprised of L-DNA. L-DNA haplomers are also advantageous in that their hybridization portions are highly resistant to all nucleases. Single strands of L-DNA are not to be confused with left-handed DNA duplexes (Z-DNA).

In some embodiments of the methods for aptamer-displayed bioorthogonal hybridization, the accessible 5′ end of a pre-defined singlet aptamer is derivatized with an L-DNA sequence tag, via mutually reactive click chemistry. A 5′ click group is introduced into the aptamer via amplification with a suitable modified top-strand primer, where the bottom-strand primer bears a 5′ biotin to facilitate generation of top (aptameric) single strands. After chemical ligation with an excess of a desired L-DNA bearing a 3′ click group (mutually reactive with the 5′ click group carried by the aptamer), the aptamer carries a 5′ tag corresponding to the desired L-DNA sequence. Upon target binding, the L-DNA tag can act as a template for haplomers, but only if these haplomers likewise carry complementary L-DNA hybridization portions.

In some embodiments of the methods for aptamer-displayed bioorthogonal hybridization, the accessible 3′ end of a singlet aptamer is derivatized with an L-DNA sequence tag, via mutually reactive click chemistry. In this case the 3′ end of a pre-defined aptamer is enzymatically ligated via RNA ligase I with a short oligonucleotide sequence (dT₆₋₈) bearing a 5′ phosphate and a 3′ click group. The aptamer 5′ end in this instance bears a 5′ hydroxyl group. Following this, chemical ligation can be carried out with an excess of a desired L-DNA bearing a 5′ click group (mutually reactive with the 3′ click group carried by the aptamer). The resulting aptamer product carries a 3′ tag corresponding to the desired L-DNA sequence. Upon target binding, the L-DNA tag can act as a template for haplomers, but only if these haplomers likewise carry complementary L-DNA hybridization portions.

In some embodiments of the methods for aptamer-displayed bioorthogonal hybridization, dual aptamers binding in spatial proximity to a designated target are used to display L-DNA templates. This approach uses appropriate Left and Right aptamers (pre-defined as binding proximally to the desired target molecule by co-binding ligation) bearing 5′ and 3′ L-DNA tags, respectively. In this instance, the haplomers with L-DNA hybridization portions are used, but haplomers are not directed solely to the 5′ end of a single aptamer or the 3′ end of a single aptamer. Instead, the haplomers are directed to the termini of each of the aptamers of the dual aptamer pair, such that bioorthogonal reactivity is promoted via spatial proximity of the dual aptamer binding on a common target molecule.

A binary aptamer can be formed from a pair of aptamers co-binding proximally close target sites on a complex molecule. The ligation of the aptamer ends upon this splint can be effected by T4 DNA ligase, or more efficiently by Chlorella DNA ligase, which is highly effective in the ligation of DNA ends by RNA splints (New England Biolabs). Following ligation, the splint can be removed with RNase H. The dotted oval indicates accessible template provided by the binary aptamers after RNA splint removal.

An unnatural L-DNA tag can be appended onto the 5′ end of a singlet aptamer. A pre-defined aptamer is re-amplified, where the top strand primer bears a 5′ click group, and the bottom strand primer bears a 5′ biotin. After amplification, single strands corresponding to the original aptamer sequence can be prepared. The resulting aptamer can then be reacted with an excess of an L-DNA tag of defined sequence, bearing a 3′-click group, orthogonally reactive with the aptameric click group. After binding its target molecule, the aptamer displays the appended L-DNA sequence as a 5′ template, which haplomers bearing complementary L-DNA hybridization portions can recognize. The curved arrows denote a proximity-induced reaction between different haplomers.

An unnatural L-DNA tag can be appended onto the 3′ end of a singlet aptamer. A pre-defined aptamer with an accessible 3′ end is ligated with a short single stranded oligonucleotide (such as dT₈) bearing a 5′ phosphate and a 3′ click group, by means of RNA ligase I. The resulting aptamer can then be reacted with an excess of an L-DNA tag of defined sequence, bearing a 5′-click group, orthogonally reactive with the aptameric click group. After binding its target molecule, the aptamer displayed the appended L-DNA sequence as a 3′ template, which haplomers bearing complementary L-DNA hybridization portions can recognize. The curved arrows denote a proximity-induced reaction between different haplomers.

Unnatural L-DNA tags can be appended onto the 3′ and 5′ ends of dual aptamers, for directing spatial proximity of haplomers by bioorthogonal hybridization. The L-DNA tags at the 3′ and 5′ ends of aptamers proximally binding the same target molecule can be appended separately as described herein.

In some embodiments of the methods for aptamer-displayed bioorthogonal hybridization, binary aptamers are used to display L-DNA templates. To achieve this, a double-derivatization process is used. Initially, singlet aptamers comprising the Left and Right segments of a binary pre-selected for proximity by co-binding are derivatized with L-DNA tags in the same manner as described herein. In this instance, the L-DNA tags also have amino groups appended to their 3′ and 5′ ends, respectively. Following the initial chemical ligations of each L-DNA tag sequence, the amino groups can be derivatized with appropriate click groups, via N-hydroxylsuccinimide chemistry. These reactions can be performed, since once the previous click groups have reacted, the products are inert towards a second derivitization. The fully derivatized L-DNA tagged aptamers can be in turn chemically ligated together by co-binding to the target molecule of interest. In this instance, the interaction between each L-DNA tag is facilitated by a short (i.e., 4-6 base) mutually complementary terminal sequence. This forms a short stem loop, which in turn facilitates the subsequent reaction of hybridizing L-DNA haplomers, by enhancing spatial proximity, as previously shown with oligonucleotides bearing click-reactive groups.

Binary aptamers can be equipped with bridging unnatural L-DNA sequences, for directing spatial proximity of haplomers by bioorthogonal hybridization. For example, Left aptamers can be prepared with derivatized L-DNA tags. The initial linkage of the L-DNA tag is as described herein, except that the L-DNA bears a 3′-amino group for a secondary derivatization with a click group. Right aptamers can be prepared with derivatized L-DNA tags. The initial linkage of the L-DNA tag is as described herein, except that the L-DNA bears a 5′-amino group for a secondary derivatization with a click group. In both cases, the secondary derivatizations can be performed since once the previous click groups have reacted, the products are inert towards a second derivitization.

Binary aptamers can also be equipped with bridging unnatural L-DNA sequences, for directing spatial proximity of haplomers by bioorthogonal hybridization. Chemical ligation on the target molecule of Left and Right derivatized aptamers bearing L-DNA sequences, and subsequent hybridization with haplomers is shown. Each Left- and Right L-DNA segment is designed to a have a short (i.e., 4-6 base) mutually complementary sequence to facilitate both local interaction and subsequent haplomers spatial proximities.

In some embodiments, the click-reactive groups can be, but are not limited to, azide and strained cyclooctyne groups, or tetrazine derivatives and trans-cyclooctene groups. For 5′ template modification, top-strand primers can be initially synthesized with a 5′ amino group, which can be subsequently converted into the appropriate click group through reaction with a click group-N-hydroxylsuccinimide moiety.

Many cases of ligand-induced allosteric structural changes have been documented with both RNA and DNA aptamers. Such effects have been usefully exploited for the generation of specific aptameric functionalities, such as aptabeacons and aptasensors. In this instance, selection for allosteric effects can be performed such that aptamer-derived template is only exposed for effector partial moiety hybridization after binding to the target molecule. Allosteric aptamers of these types add additional power to the utility of aptamers as display vehicles for template assembly. Specifically, an aptamer system where the accessible template is only exposed after target molecule binding promises to reduce non-specific haplomer interactions. In other words, in an environment where the aptamer encounters no specific target, no template is accessible for templated assembly either.

In some embodiments for the methods for selection of allosteric aptamers for haplomer applications, a singlet aptamer is selected where the terminal template sequence for template assembly is only exposed and accessible after aptamer binding to the specific target molecule. This process involves a cycle of negative and positive selections. The first step involves partitioning an unselected aptamer library into those members with accessible termini in solution, and those members whose termini are not accessible to hybridization, as defined with a biotinylated probe sequence. Solution-accessible members of the library can be removed by binding of the annealed probe to, for example, a solid-phase streptavidin matrix. This process accordingly negatively selects for folded aptamers in solution whose template sequences are not accessible to an added probe molecule. Within this population, a second positive selection (again by means of, for example, a biotinylated probe sequence) can be made for members which generate accessible template sequences as a consequence of target binding. This positive selection is analogous to the selection process for singlet aptamers with accessible targets. When resulting selected aptamers are amplified and the appropriate single-stranded preparations made, the process can be repeated as a cycle. When the heterogeneity of the selected population after N cycles is highly reduced, the resulting population can be cloned and individual aptamers screened. Candidate aptamers can conform to the original selection criteria as being refractory to template-based interactions in free solution, but amenable to such interactions in the presence of specific template. Allosterically-induced accessible template can also permit the templated assembly of haplomers.

A selection process for aptamer allostery, where target molecule binding induces the exposure and/or accessibility of the template sequence can also be carried out. Aptamers whose templating sequences are accessible in solution (before presence of a target molecule) can be removed by initial hybridization to, for example, an appropriate biotinylated probe sequence, and immobilization on, for example, a solid-phase streptavidin matrix. The supernatant fraction can be incubated with target molecule in solution. Aptamers which bind to the target molecule and undergo an allosteric change which renders the template sequence accessible are selectable by, for example, biotinylated probe binding. Those aptamers whose template sequences remain masked or inaccessible are not. Therefore, sequestration of the former on, for example, a solid-phase streptavidin matrix allows their selective amplification. The eluted aptamer preparations obtained in this manner can be then subjected to a repeat of the whole cycle. Cycling can be performed until analyses of the resulting populations show highly reduced homogeneity, after which analysis of specific cloned aptamers can be carried out.

In some embodiments of the methods for selection of allosteric aptamers for effector partial moiety applications, binary aptamers are selected where the 3′ and 5′ ends of each singlet component comprising the binary form are only exposed in accessible proximity following target molecule binding. Both Left and Right aptamers directed towards a target of interest can be initially derived in the same manner as the methods described herein, where both aptamers exhibit allosterically exposable template sequences only following interaction with a target molecule. Populations of such target-directed aptamers can be subjected to the co-binding process and splint-directed in situ ligation. Specific binary pairs can be identified and characterized by amplifying the proximal binary units as a single contiguous sequence. When specific pairs are identified, splint removal can be effected by using RNase H, after which the junctional template sequence is available for haplomer templated assembly.

Aptamer allostery towards the in situ generation of joined binaries is also possible. The linking templating sequences between each Left and Right component of a binary aptamer pair are only available following target molecule binding and allosteric exposure of terminal templates in spatial proximity Such pairs can be identified by co-binding on the original target, RNA splint-mediated ligation, and amplification. Once a specific binary aptamer pair have been identified, they can be used for haplomer templating in the same manner as detailed herein.

In some embodiments, an initial round of Left- and Right-aptamer selection for binders is performed using the tumor-derived source material of interest. If the source material is whole cells, unbound material can be removed during binding selection by low-speed centrifugation and washing. If the source material is whole-cell cytoplasmic lysate or whole cell RNA, unbound material can be removed by, for example, differential PEG precipitation. This step can be followed by a subtractive removal of aptamers binding to material from cognate normal sources, where the separation of bound and unbound is the same as in the initial step. These steps can be repeated through a series of cycles as appropriate (10 such cycles are usually sufficient).

In particular, some methods involve subtraction between aptamers binding targets from a tumor cell source and those binding a matched cognate normal cell. The source material can be whole cells (selecting for cell-surface targets), whole cell cytoplasmic lysates (selecting for all intracellular targets, including protein, RNA, and ribonucleoproteins), or whole RNA. L- and R-aptamer libraries can be initially used to select subpopulations which bind to tumor sources, and which escape removal by binding to corresponding normal source targets, in order to enrich for aptamers exclusively binding tumor-related molecules. This binding and subtraction process can be repeated for a suitable number of cycles. L- and R-aptamer libraries directly binding such normal counterparts to the tumors can also be directly selected for the next stage of the process, using the same number of cycles.

In a variation of such methods, the normal source material of interest (corresponding to the tumor source material) can also be used to select for binding Left- and Right-aptamer populations directly, where the separation of bound and unbound is the same as described above. The resulting subpopulations of Left- and Right-aptamers binding normal target molecules can be used for the subsequent selective purposes.

Following the steps outlined above after appropriate cycling, the selected subpopulations of Left- and Right-aptamers binding tumor sources of interest and subtracted for cognate normal sources can be used for co-binding experiments on the same original tumor sources. L(TΔN) and R(TΔN) denote Left-aptamers binding tumor sources of interest and subtracted for cognate normal sources, and Right-aptamers binding tumor sources of interest and subtracted for cognate normal sources, respectively. In addition, it can be useful to perform tests with both L(TΔN) and R(TΔN) subpopulations co-bound to a tumor target in conjunction with corresponding Right- and Left-aptamers previously selected for binding to cognate normal targets (R(N) and L(N), respectively). Co-binding experiments with L- and R-tumor-binding, normal source-subtracted subpopulations can be performed, but also with each of these L- and R-populations co-bound with R- and L-subpopulations (respectively) from corresponding normal sources. The use of “half-normal” binaries is for increasing the probability of finding an amplifiable binary product where at least one half has tumor specificity.

A rationale for the subtractive/co-binding processes can be derived from the unknown surface density of novel tumor-specific targets. While a binary composed of both Left- and Right-tumor-restricted epitopes is desired, a singlet epitope that defines a tumor subset is still very valuable. An aptamer recognizing such an epitope in conjunction with a proximal normal epitope retains its ability to recognize the target tumor cell, but also gains the improvements in specificity and affinity associated with binary aptamers.

In some embodiments, the subtraction process involves tumor target cells with and without an in vitro drug treatment. Here, drug-treated whole tumor cells or treated tumor cell extracts can be used to select for L- and R-aptamer binders, and corresponding untreated tumor cells likewise subjected to the same selection processes. For each cycle of selection for the drug-treated cohort, aptamers binding untreated cells or untreated extracts can be removed. Finally, co-binding selection for binary aptamers binding treated tumor targets can be performed, where either or both of the L- and R-components exclusively bind to the treated preparations, analogously as for tumor/normal cells.

Testing of the efficacy of templating of epitope haplomers for eliciting recognition by antibodies or other recognition molecules can be initially demonstrated in vitro for all embodiments, in advance of in vivo applications. Such assessment is best made by ELISA assays, where a biotinylated template nucleic acid strand is surface-immobilized by means of streptavidin (SA). Pairs of epitope haplomers and suitable controls are then hybridized with the surface templates, followed by incubation with the recognition molecule (often an antibody) of interest. The final read-out is effected in various ways, including (but not limited to) the use of a secondary antibody coupled with horseradish peroxidase, for visualization of enzymatic activity via standard development reagents.

In some embodiments, aptamer-mediated templating of effector partial moieties directs the assembly of peptide epitopes recognized by well-characterized therapeutic antibodies. Such antibodies include, but are not limited to, antibodies recognizing HER-2/neu, EGFR, and VEGF. Where short peptide sequences corresponding to the recognition sites on the target antigens are not available, this embodiment also includes peptide epitope identification with the available antibodies of interest by means of, for example, peptide phage display libraries, as employed in the identification of lymphoma antibody binding specificities. Additional antibodies as recognition molecules include, for example, palivizumab, motavizumab, panitumumab, metuximab, antibodies to bacterial antigens, antibodies to viral antigens, and antibodies to parasitic antigens.

An example of a mimotope for trastuzumab is a peptide with the sequence QLGPYEL WELSH (SEQ ID NO:36), a derivative of the initial mimotope identification (LLGPYELWEL SH; SEQ ID NO:37) with higher binding affinity by virtue of the L1 Q substitution. In some embodiments, the epitope is a polypeptide comprising the formula: SerGlyGlyGlySerGlyGlyGly GlnLeuXaa¹ProTyrGluXaa²TrpGluLeuXaa³His, wherein one of: a) Xaa¹ is Cys, Xaa² is Leu, and Xaa³ is Ser (SEQ ID NO:1); b) Xaa¹ is Gly, Xaa² is Cys, and Xaa³ is Ser (SEQ ID NO:2); or c) Xaa¹ is Gly, Xaa² is Leu, and Xaa³ is Cys (SEQ ID NO:3). In some embodiments, the epitope is a polypeptide comprising the formula: SerGlyGlyGlySerGlyGlyGlyGlnLeuXaa¹Pro TyrGluXaa²TrpGluLeuXaa³His (SEQ ID NO:3) (i.e., termed “S11C”), wherein Xaa¹ is Gly, Xaa² is Leu, and Xaa³ is Cys. In some embodiments, the N-terminus of the polypeptide comprises a biotin.

In some embodiments, the epitope is a polypeptide comprising the formula: SerGlyGly GlySerGlyGlyGlyGlnXaa¹LeuXaa²GlyXaa³ProXaa⁴TyrXaa⁵GluXaa⁶LeuXaa⁷TrpXaa⁸GluXaa⁹LeuXaa¹⁰SerXaa¹¹His, wherein one of: a) Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent (SEQ ID NO:4); b) Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent (SEQ ID NO:5); c) Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent (SEQ ID NO:6); d) Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent (SEQ ID NO:7); e) Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent (SEQ ID NO:8); f) Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent (SEQ ID NO:9); g) Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent (SEQ ID NO:10); h) Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent (SEQ ID NO:11); i) Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are absent (SEQ ID NO:12); j) Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent (SEQ ID NO:13); or k) Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent (SEQ ID NO:14). In some embodiments, the N-terminus of the polypeptide comprises a biotin.

In some embodiments, chosen epitopes are functionally split by screening for individual residues whose replacement by cysteine residues is tolerated. As a non-limiting example with the trastuzumab mimotope QLGPYELWELSH (SEQ ID NO:36), the following peptides are synthesized and screened for retention of binding to trastuzumab: 1) (N-terminus) Biotin-SGGG SGGGQLCPYELWELSH (SEQ ID NO:1); 2) (N-terminus) Biotin-SGGGSGGGQLGPYECW ELSH (SEQ ID NO:2); and 3) (N-terminus) Biotin-SGGGSGGGQLGPYELWELCH (SEQ ID NO:3). In addition, mimotope derivatives may be screened where cysteine residues are progressively inserted into the mimotope sequence, rather than via residue replacement. As a non-limiting example, the following peptide is synthesized and screened for retention of binding to trastuzumab: 4) (N-terminus) Biotin-SGGGSGGGQLGPYECLWELSH (SEQ ID NO:9). The N-terminal biotinylation and serine-glycine linkers are present to provide optimal signals from ELISA assays, as performed with the parental mimotope of the sequence: (N-terminus) biotin-SGGGSGGGQLGPYELWELSH (SEQ ID NO:35).

In some embodiments, the recognition molecule is palivizumab, which is a humanized monoclonal antibody which binds to the F glycoprotein of Respiratory Syncytial Virus (RSV), and blocks viral cell entry. In some embodiments, the recognition molecule is motavizumab, which is a derivative of palivizumab with increased affinity. An example of an epitope for palivizumab and motavizumab is a helix-loop-helix motif, where two alpha helices are separated by a short (4-residue) loop segment. Important contact residues towards the antibody combining site are within both helices, while the loop region does not contribute to motavizumab binding. The native sequence of the epitope recognized by these antibodies is: NSELLSLINDMPITNDQ KKLMSNN (SEQ ID NO:38), where the helices are bolded and the loop region is between the bolded helices. There is a significant loss of antibody binding towards the free peptide epitope in solution compared to within the natural F protein itself, attributed at least in part to poor adoption of the helix-loop-helix conformation by isolated peptide. Therefore, peptides for split epitope work are designed (where possible) with enhanced N-cap and C-cap residues.

In some embodiments, the N-terminal epitope fragment for palivizumab/motavizumab comprises the sequence: NSELLSLIND-MGPSGGGS-(Azide) (SEQ ID NO:39), where the azide group is present to facilitate conjugation to oligonucleotides bearing 5′ or 3′ click groups with complementary reactivity, including, but not limited to, dibenzylcyclooctyne (DBCO). The introduced GPS enhanced C-cap is in boldface. In some embodiments, the N-terminal epitope fragment comprises an additional two residues at its N-terminus, to enhance helicity. These also correspond to the two residues immediately N-terminal to the epitope in the context of native RSV F protein. The alternate sequence is: LTNSELLSLIND-MGPSGGGS-(Azide) (SEQ ID NO:40), where the introduced N-terminal (LT) residues are LT. In some embodiments, alternate sequences at the C-terminus of the NSELLSLIND (SEQ ID NO:41) epitope fragment sequence are used, including, but not limited to: MPITSGGGS-(Azide) (SEQ ID NO:42), MGGSSGGGS-(Azide) (SEQ ID NO:43), MGAPSGGGS-(Azide) (SEQ ID NO:44), GGPSSGGGS-(Azide) (SEQ ID NO:45), and GPSGSGGGS-(Azide) (SEQ ID NO:46).

In some embodiments, the C-terminal epitope fragment for palivizumab/motavizumab comprises the sequence: (Azide)-SGGGGLS-NDQKKLMSNN (SEQ ID NO:47), where the azide group is present to facilitate conjugation to oligonucleotides bearing 5′ or 3′ click groups with complementary reactivity, including, but not limited to, dibenzylcyclooctyne (DBCO). The introduced GLS enhanced N-cap is in boldface. In some embodiments, the C-terminal epitope fragment possesses an additional two residues at its C-terminus, to enhance helicity. These also correspond to the two residues immediately C-terminal to the epitope in the context of native RSV F protein. The alternate sequence is: (Azide)-SGGGGLS-NDQKKLMSNNVQ (SEQ ID NO:48), where the introduced C-terminal residues are VQ. In some embodiments, alternate sequences at the N-terminus of the NDQKKLMSNN (SEQ ID NO:49) epitope fragment sequence are used, including, but not limited to: (Azide)-SGGGGLS (SEQ ID NO:50), (Azide)-SGGGGAS (SEQ ID NO:51), (Azide)-SGGGGAP (SEQ ID NO:52), (Azide)-SGGGGLD (SEQ ID NO:53), and (Azide)-SGGGGLN (SEQ ID NO:54).

In some embodiments, the recognition molecule is panitumumab, which is an antibody which binds to Epidermal Growth Factor Receptor (EGFR). Numerous peptide sequences of EGF include, but are not limited to: IYPPLLRTSQAM (SEQ ID NO:55), AYPPYLRSMTLY (SEQ ID NO:56), YPPAERTYSTNY (SEQ ID NO:57), CPKWDAARC (SEQ ID NO:58), and CGPTRWRSC (SEQ ID NO:59).

In some embodiments, the recognition molecule is ATVi, a monoclonal antibody which binds to the Vi antigen of Salmonella enterica. Numerous peptide sequences the Vi antigen include, but are not limited to: TSHHDSHGLHRV (SEQ ID NO:60), TSHHDSHGDHHV (SEQ ID NO:61), TSHHDSHGVHRV (SEQ ID NO:62), TSHHDSHDLHRV (SEQ ID NO:63), TSHH DYHGLHRV (SEQ ID NO:64), ENHSPVNIAHKL (SEQ ID NO:65), ENHSPVNIAHKV (SEQ ID NO:66), ENHSPVNIDHKL (SEQ ID NO:67), EDHSPVNIDHKL (SEQ ID NO:68), ENHYP LHAAHRI (SEQ ID NO:69), ESHQHVHDLVFL (SEQ ID NO:70), PGHHDFVGLHHL (SEQ ID NO:71), ENHYPVNIAHKL (SEQ ID NO:72), and DNHSPVNIAHKL (SEQ ID NO:73).

In some embodiments, the recognition molecule is AVFluIgG01, a human monoclonal antibody which binds to the H5N1 Influenza Virus. Numerous peptide sequences of H5N1 include, but are not limited to: YINPHMYWMSVA (SEQ ID NO:74), HTPPPQPYRTHI (SEQ ID NO:75), TFWVQTAKPNPL (SEQ ID NO:76), GHPSKTSGHPLT (SEQ ID NO:77), TYVN IVLYDDVE (SEQ ID NO:78), TTNFLNHAIAHK (SEQ ID NO:79), YYNPSPPNPRTQ (SEQ ID NO:80), TESPQYIALSFH (SEQ ID NO:81), HWYDWLTRYSHL (SEQ ID NO:82), AT YTTDAQSYHM (SEQ ID NO:83), DHYWHRSNTLSH (SEQ ID NO:84), VTSHDLKKSG TW (SEQ ID NO:85), WEFAYKNTRYYW (SEQ ID NO:86), SWTSLPLHEAIH (SEQ ID NO:87), TLAHTHTSTSSF (SEQ ID NO:88), WHWSFFASPLPA (SEQ ID NO:89), WHW NARNWSSQQ (SEQ ID NO:90), CWTSLPLHEAIH (SEQ ID NO:91), VPTECSGRTSCT (SEQ ID NO:92), WSNHWWHSKWAI (SEQ ID NO:93), HIWNWSNWTQWT (SEQ ID NO:94), HIFHNTHWWQRW (SEQ ID NO:95), TNYDYIPDTQNT (SEQ ID NO:96), SWS SHSNSTPTSYNTNQTQNPTSTSTNQPNNN (SEQ ID NO:97), and NHEKIPKSSWSSHWK YNTNQEDNKTIKPNDNEYKVK (SEQ ID NO:98).

In some embodiments, the recognition molecule is metuximab (LICARTIN®), which is an antibody which binds to CD147. Numerous peptide sequences of CD147 include, but are not limited to: YPHFHKHTLRGH (SEQ ID NO:99), YPHFHKHSLRGQ (SEQ ID NO:100), DHK PFKPTHRTL (SEQ ID NO:101), FHKPFKPTHRTL (SEQ ID NO:102), QSSCHKHSVRGR (SEQ ID NO:103), QSSFSNHSVRRR (SEQ ID NO:104), and DFDVSFLSARMR (SEQ ID NO:105).

In some embodiments, the recognition molecule is 152-66-9b, which is a monoclonal antibody which binds to Schistosoma mansoni. Numerous peptide sequences of Schistosoma mansoni include, but are not limited to: VLLRRIGG (SEQ ID NO:106), HLLRLSEI (SEQ ID NO:107), SLLTYMKM (SEQ ID NO:108), and YLLQKLRN (SEQ ID NO:109).

Additional epitopes for a wide variety of recognition molecules are know in in the art and can be employed in any of the methods disclosed herein.

To be useful in the split-epitope methodology disclosed herein, each segment of an epitope (i.e., each reactive effector moiety forming portions of haplomer pairs that, when combined, form an epitope for a recognition molecule) should lack significant binding to antibody alone, but activity should be conferred via their mutual forced proximity or through their covalent rejoining. In some embodiments, an epitope can be altered by replacing a serine with a cysteine. In some embodiments, only one cysteine is inserted or substituted into an epitope.

In some embodiments the SerGlyGlyGlySerGlyGlyGly (SEQ ID NO:110) portion of any of the epitopes described herein can be altered. In some embodiments, the SerGlyGlyGly SerGlyGlyGly (SEQ ID NO:110) portion of the epitope can be of variable length, for example, SerGlyGlyGly (SEQ ID NO:111). In some embodiments, the SerGlyGlyGlySerGlyGlyGly (SEQ ID NO:110) portion of the epitope can be comprised of other amino acids such as, for example, threonines, glutamines, and asparagines. In some embodiments, the SerGlyGlyGlySerGlyGlyGly (SEQ ID NO:110) portion of the epitope can be replaced with SerGlyGlySerSerGlyGly (SEQ ID NO:112).

In some embodiments, the biotin functions as an anchor for either ELISA studies or on-cell studies. In some embodiments, the biotin can be replaced with other known suitable molecules.

In some embodiments, structural information from antibody:target antigen complexes is used to choose the site of cleavage of an epitope into two segments. This is particularly so when the epitope is known to consist of two or more discontiguous components, as exemplified by a natural epitope with the configuration: A1A2A3 . . . An-xxxxxxxx-B1B2B3 . . . Bn, where A1-An and B1-Bn are tracts of residues making contact with the antibody recognition site, and x denotes an extended tract which does not make effective antibody contact. In such cases, the epitope is discontiguous over the sequence A1-Bn with a defined non-contact region. As such, in these circumstances, it is logical to split the epitope into two fragments: A1A2A3 . . . An-515253 . . . Sn-V and W-S1S2S3 . . . Sn-B1B2B3 . . . Bn, where S1S2S3 . . . Sn constitute residues of a linker sequence, usually, but not limited to, a composition of serine and glycine residues. One purpose of the linker sequence is to spatially position the respective (A) and (B) epitope segments in a manner favorable for binding to the antibody of interest. In turn, the length of the required linker sequence can also be assessed, within definable limits, from structural information if available.

After spatial proximity of the two split epitope segments (bearing bio-orthogonally reactive chemical groups V and W) is achieved by mutual templating, specific chemical reactivity results in reconstitution of an antibody-reactive epitope: A1A2A3 . . . An-SE.Sn-[R]-S1 . . . Sn-B1B2B3 . . . Bn, where R is the chemical residue resulting from the reaction between the mutually bio-orthogonally reactive groups V and W.

The mutually bio-orthogonally reactive groups V and W can be constituted by pairs of click reagents, including, but not limited to, linear alkyne/azide, strained alkyne/azide, and tetrazine/cyclooctene pairs. Additional bio-orthogonally reactive groups are described herein.

Where a cysteine replacement or insertion within epitope sequence is found to be compatible with antibody recognition (between 50-100% of the binding capacity of the antibody of interest towards unmodified epitope, as assessed by comparative ELISA titers), then the cysteine-bearing epitope can be functionally dissected at the cysteine site. Two fragments thus constituting the split epitope can be produced and reassembled when in spatial proximity by means of native chemical ligation (NCL). A non-limiting example of this is provided with the peptide sequence 1) above: 1) (N-terminus) biotin-SGGGSGGGQLCPYELWELSH (SEQ ID NO:1); split peptides correspond to: 1a) SGGGSGGGQL-(C-terminal phenyl thioester) (SEQ ID NO:15) and 1b) CPYELWELSH (SEQ ID NO:16). Another example where the epitope is a polypeptide comprising the formula: SerGlyGlyGlySerGlyGlyGlyGlnLeuXaa¹ProTyrGluXaa²TrpGluLeuXaa³His (SEQ ID NO:3) (i.e., termed “S11C”), wherein Xaa¹ is Gly, Xaa² is Leu, and Xaa³ is Cys, the resulting split peptides (which are the reactive effector molecules for their haplomers) are: SGGGQLCPYELWEL-(C-terminal phenyl thioester) (SEQ ID NO:113) and CHGGGS (SEQ ID NO:114).

In some embodiments, the peptides may be further modified for native chemical ligation (NCL)-mediated epitope reconstitution to allow their conjugation with oligonucleotides, such that they can be rendered as epitope haplomers. In some embodiments, a peptide can be modified to add N- or C-terminal azide groups. For N-terminal azides, the modification can be achieved by incorporation of an N-terminal azidoacetic group. For C-terminal azides, the modification can be achieved by means of a C-terminal azidolysine residue. In some embodiments, azide-modified peptides are reacted with oligonucleotides modified at their 3′ or 5′ ends with dibenzylcyclooctyne (DBCO) groups for copper-free ‘strained click’ reaction. In some embodiments, azide-modified peptides are reacted with oligonucleotides modified at their 3′ or 5′ ends with linear alkynes, in the presence of Cu(I) catalysts.

In some embodiments, pairs of reactive effector moieties (e.g., pairs of polypeptides that can form an epitope upon templated assembly, or compositions thereof) for trastuzumab include, but are not limited to: a) SerGlyGlyGlySerGlyGlyGlyGlnLeu (SEQ ID NO:15) and Xaa¹ProTyr GluXaa²TrpGluLeuXaa³His (SEQ ID NO:16), wherein Xaa¹ is Cys, Xaa² is Leu, and Xaa³ is Ser; b) SerGlyGlyGlySerGlyGlyGlyGlnLeuXaa¹ProTyrGlu (SEQ ID NO:17) and Xaa²TrpGlu LeuXaa³His (SEQ ID NO:18), wherein Xaa¹ is Gly, Xaa² is Cys, and Xaa³ is Ser; and c) SerGly GlyGlySerGlyGlyGlyGlnLeuXaa¹ProTyrGluXaa²TrpGluLeu (SEQ ID NO:19) and Xaa³His, wherein Xaa¹ is Gly, Xaa² is Leu, and Xaa³ is Cys.

In some embodiments:

a) one of the reactive effector moiety of the first epitope haplomer and the reactive effector moiety of the second epitope haplomer is SerGlyGlyGlySerGlyGlyGlyGlnLeu (SEQ ID NO:15) and the other of the reactive effector moiety of the first epitope haplomer and the reactive effector moiety of the second epitope haplomer is Xaa¹ProTyrGluXaa²TrpGluLeuXaa³His (SEQ ID NO:16), wherein Xaa¹ is Cys, Xaa² is Leu, and Xaa³ is Ser;

b) one of the reactive effector moiety of the first epitope haplomer and the reactive effector moiety of the second epitope haplomer is SerGlyGlyGlySerGlyGlyGlyGlnLeuXaa¹ProTyrGlu (SEQ ID NO:17) and the other of the reactive effector moiety of the first epitope haplomer and the reactive effector moiety of the second epitope haplomer is Xaa²TrpGluLeu Xaa³His (SEQ ID NO:18), wherein Xaa¹ is Gly, Xaa² is Cys, and Xaa³ is Ser;

c) one of the reactive effector moiety of the first epitope haplomer and the reactive effector moiety of the second epitope haplomer is SerGlyGlyGlySerGlyGlyGlyGlnLeuXaa¹ProTyrGluXaa²TrpGluLeu (SEQ ID NO:19) and the other of the reactive effector moiety of the first epitope haplomer and the reactive effector moiety of the second epitope haplomer is Xaa³His, wherein Xaa¹ is Gly, Xaa² is Leu, and Xaa³ is Cys;

d) one of the reactive effector moiety of the first epitope haplomer and the reactive effector moiety of the second epitope haplomer is SerGlyGlyGlySerGlyGlyGlyGln (SEQ ID NO:20) and the other of the reactive effector moiety of the first epitope haplomer and the reactive effector moiety of the second epitope haplomer is Xaa¹LeuXaa²GlyXaa³ProXaa⁴Tyr Xaa⁵GluXaa⁶LeuXaa⁷TrpXaa⁸GluXaa⁹LeuXaa¹⁰SerXaa¹ (SEQ ID NO:21), wherein Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent;

e) one of the reactive effector moiety of the first epitope haplomer and the reactive effector moiety of the second epitope haplomer is SerGlyGlyGlySerGlyGlyGlyGlnXaa¹Leu (SEQ ID NO:15) and the other of the reactive effector moiety of the first epitope haplomer and the reactive effector moiety of the second epitope haplomer is Xaa²GlyXaa³ProXaa⁴TyrXaa⁵Glu Xaa⁶LeuXaa⁷TrpXaa⁸GluXaa⁹LeuXaa¹⁰SerXaa¹¹His (SEQ ID NO:22), wherein Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent;

f) one of the reactive effector moiety of the first epitope haplomer and the reactive effector moiety of the second epitope haplomer is SerGlyGlyGlySerGlyGlyGlyGlnXaa¹Leu Xaa²Gly (SEQ ID NO:23) and the other of the reactive effector moiety of the first epitope haplomer and the reactive effector moiety of the second epitope haplomer is Xaa³ProXaa⁴Tyr Xaa⁵GluXaa⁶LeuXaa⁷TrpXaa⁸GluXaa⁹LeuXaa¹⁰SerXaa¹¹His (SEQ ID NO:16), wherein Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent;

g) one of the reactive effector moiety of the first epitope haplomer and the reactive effector moiety of the second epitope haplomer is SerGlyGlyGlySerGlyGlyGlyGlnXaa¹Leu Xaa²GlyXaa³Pro (SEQ ID NO:24) and the other of the reactive effector moiety of the first epitope haplomer and the reactive effector moiety of the second epitope haplomer is Xaa⁴Tyr Xaa⁵GluXaa⁶LeuXaa⁷TrpXaa⁸GluXaa⁹LeuXaa¹⁰SerXaa¹¹His (SEQ ID NO:25), wherein Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent;

h) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴Tyr (SEQ ID NO:26) and the other of the reactive effector moiety of the first epitope haplomer and the reactive effector moiety of the second epitope haplomer is Xaa⁵GluXaa⁶LeuXaa⁷TrpXaa⁸GluXaa⁹LeuXaa¹⁰SerXaa¹¹His (SEQ ID NO:27), wherein Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent;

i) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵Glu (SEQ ID NO:17) and the other of the reactive effector moiety of the first epitope haplomer and the reactive effector moiety of the second epitope haplomer is Xaa⁶LeuXaa⁷TrpXaa⁸GluXaa⁹LeuXaa¹⁰SerXaa¹¹His (SEQ ID NO:28), wherein Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent;

j) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶Leu (SEQ ID NO:29) and the other of the reactive effector moiety of the first epitope haplomer and the reactive effector moiety of the second epitope haplomer is Xaa⁷TrpXaa⁸GluXaa⁹LeuXaa¹⁰SerXaa¹¹His (SEQ ID NO:18), wherein Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent;

k) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶LeuXaa⁷Trp (SEQ ID NO:30) and the other of the reactive effector moiety of the first epitope haplomer and the reactive effector moiety of the second epitope haplomer is Xaa⁸GluXaa⁹LeuXaa¹⁰SerXaa¹¹His (SEQ ID NO:31), wherein Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent;

l) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶LeuXaa⁷TrpXaa⁸Glu (SEQ ID NO:32) and the other of the reactive effector moiety of the first epitope haplomer and the reactive effector moiety of the second epitope haplomer is Xaa⁹LeuXaa¹⁰SerXaa¹¹His (SEQ ID NO:33), wherein Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are absent;

m) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶LeuXaa⁷TrpXaa⁸GluXaa⁹Leu (SEQ ID NO:19) and the other of the reactive effector moiety of the first epitope haplomer and the reactive effector moiety of the second epitope haplomer is Xaa¹⁰SerXaa¹¹His, wherein Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent; or

n) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶LeuXaa⁷TrpXaa⁸GluXaa⁹LeuXaa¹⁰Ser (SEQ ID NO:34) and the other of the reactive effector moiety of the first epitope haplomer and the reactive effector moiety of the second epitope haplomer is Xaa¹¹His, wherein Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent.

In some embodiments, the C-terminus of the first polypeptide further comprises a first bio-orthogonal reactive group and the N-terminus of the second polypeptide further comprises a second bio-orthogonal reactive group, wherein the first bio-orthogonal reactive group and the second bio-orthogonal reactive group are compatible.

In some embodiments, the first bio-orthogonal reactive group is a linear alkyne and the second bio-orthogonal reactive group is an azide, or the second bio-orthogonal reactive group is a linear alkyne and the first bio-orthogonal reactive group is an azide; the first bio-orthogonal reactive group is a strained alkyne and the second bio-orthogonal reactive group is an azide or the second bio-orthogonal reactive group is a strained alkyne and the first bio-orthogonal reactive group is an azide; or the first bio-orthogonal reactive group is a tetrazine and the second bio-orthogonal reactive group is a cyclooctene or the second bio-orthogonal reactive group is a tetrazine and the first bio-orthogonal reactive group is a cyclooctene.

In some embodiments, the C-terminus of the first polypeptide further comprises a first chemical modification and the N-terminus of the second polypeptide further comprises a second chemical modification, wherein the chemical modification and the second chemical modification are compatible.

In some embodiments, the first chemical modification is amidation (CONH₂) or esterification (COOR), where R is methyl, ethyl, or phenyl; and the second chemical modification is acetylation or an N-methyl substitution of the N-terminal amino group.

In some embodiments, two epitope fragments are brought into spatial proximity by mutual hybridization of conjugated nucleic acid sequences with a common template. The epitope segment(reactive effector moiety)—nucleic acid conjugates are referred to herein as “epitope haplomers.” Conjugation between synthesized epitope segments and desired nucleic acid sequences can be effected in a number of ways, including, but not limited to, reactions between terminal thiol groups (such as reduced cysteine residues) and maleimide-based cross-linkers, reactions between strained-alkyne click chemistry groups, and reactions between chemical groups participating in inverse-electron demand Diels-Alder click chemistry. An examplary coupling method using a bis-maleimide (PEG)₂ compound (BMP2, Sigma), to form a covalent linkage between a 5′ or 3′ thiol on an oligonucleotide and a thiol from a reduced cysteine residue on a peptide is described herein.

In some embodiments, it is necessary to purify the conjugation products (epitope haplomers) arising from chemical reaction between epitope segments and nucleic acids, to remove unreacted material. This can be effected by various strategies, including (but not limited to) gel electrophoreses, size-exclusion chromatography, and HPLC approaches.

Suitable target cells include any cell that is desired to be targeted including, but not limited to, cancer cells and virus-infected cells.

In some embodiments, the target cell is a pathogenic cell which is infected by a virus. The templated method, and administration of a suitable recognition molecule to the assembled epitope, may produce at least one of programmed cell death of the virus infected cell, apoptosis of the virus infected cell, non-specific or programmed necrosis of the virus infected cell, lysis of the virus infected cell, inhibition of viral infection, and inhibition of viral replication. In some embodiments, viral-specific targets can be intracellular viral transcripts or host transcripts induced into abnormal expression patterns as a consequence of viral infection, or surface structures also manifested as a result of viral replication. Non-limiting examples of the latter include abnormal surface expression of phospholipids such as phosphatidylserine.

In some embodiments, the pathogenic cell is a microbe-infected cell. The templated method, and administration of a suitable recognition molecule to the assembled epitope, may produce at least one of programmed cell death of the microbe-infected cell, apoptosis of the microbe-infected cell, non-specific or programmed necrosis of the microbe-infected cell, lysis of the microbe-infected cell, inhibition of microbial infection, and inhibition of microbe replication.

In some embodiments, various other pathogenic cells are targeted. These include, but are not limited to, pathogenic immune cells or immune cells whose removal is beneficial to a human or animal. In such cases, specific molecular targets include, but are not limited to, idiotypic domains of antibody or T cell receptors of clonal B or T cells respectively, cell lineage-specific surface markers, and cell lineage-specific cytokines.

In some embodiments, the pathogenic cell is a tumor or cancer cell. The templated method, and administration of a suitable recognition molecule to the assembled epitope, may produce at least one of programmed cell death of the tumor or cancer cell, apoptosis of the tumor or cancer cell, non-specific or programmed necrosis of the tumor or cancer cell, lysis of the tumor or cancer cell, inhibition of the tumor or cancer cell growth, inhibition of oncogene expression in the tumor or cancer cell, and modification of gene expression in the or cancer tumor cell.

Representative tumor or cancer cells include, but are not limited to: acute lymphoblastic leukemia (ALL), acute myeloid leukemia (AML), adrenocortical carcinoma, Kaposi sarcoma, lymphoma, anal cancer, astrocytoma, atypical teratoid/rhabdoid tumor, basal cell carcinoma, bile duct cancer, bladder cancer, bone cancer (Ewing sarcoma, osteosarcoma, and malignant fibrous histiocytoma), brain tumor, breast cancer, bronchial tumor, Burkitt lymphoma, non-Hodgkin lymphoma, carcinoid tumor, cardiac tumor, embryonal tumor, germ cell tumor, cervical cancer, cholangiocarcinoma, chordoma, chronic lymphocytic leukemia (CLL), chronic myelogenous leukemia (CML), chronic myeloproliferative neoplasm, colorectal cancer, craniopharyngioma, cutaneous T-cell lymphoma (mycosis fungoides and Sézary syndrome), ductal carcinoma in situ (DCIS), embryonal tumor, endometrial cancer, uterine cancer, ependymoma, esophageal cancer, esthesioneuroblastoma, extracranial germ cell tumor, extragonadal germ cell tumor, eye cancer, childhood intraocular melanoma, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor (GIST), testicular cancer, gestational trophoblastic disease, hairy cell leukemia, head and neck cancer, hepatocellular cancer, histiocytosis, Hodgkin lymphoma, hypopharyngeal cancer, islet cell tumors, pancreatic neuroendocrine tumor, kidney (renal cell) cancer, laryngeal cancer, papillomatosis, lip and oral cavity cancer, liver cancer, lung cancer (non-small cell and small cell), male breast cancer, Merkel cell carcinoma, mesothelioma, malignant childhood mesothelioma, metastatic cancer, metastatic squamous neck cancer, midline tract carcinoma, mouth cancer, multiple endocrine neoplasia syndrome, multiple myeloma/plasma cell neoplasm, myelodysplastic syndrome, myelodysplastic/myeloproliferative neoplasm, nasal cavity and paranasal sinus cancer, nasopharyngeal cancer, neuroblastoma, oral cancer, ovarian cancer, pancreatic cancer, paraganglioma, parathyroid cancer, penile cancer, pharyngeal cancer, pheochromocytoma, pituitary tumor, plasma cell neoplasm, pleuropulmonary blastoma, primary peritoneal cancer, prostate cancer, rectal cancer, rhabdomyosarcoma, salivary gland cancer, vascular tumor, uterine sarcoma, small intestine cancer, soft tissue sarcoma, squamous cell carcinoma, stomach cancer, T-cell lymphoma, testicular cancer, throat cancer, thymoma, thymic carcinoma, thyroid cancer, transitional cell cancer of the renal pelvis and ureter, urethral cancer, vaginal cancer, and Wilms tumor. Each of these types of cancer cells can be targeted via a target molecule, disirably unique to the cancer cell, for templated assembly of an epitope for a therapeutic recognition molecule.

In some embodiments, tumor-specific target molecules for aptamer-based template assembly can be uncharacterized, especially as individual tumors that undergo progressive evolutionary changes in vivo, associated with increasing tumor heterogeneity. Here, novel aptameric targets can be isolated by physical subtractive approaches, by means of matched normal cells of equivalent lineages. Initially, a specific tumor cell type of interest is used, and also a matched normal control cell type for subtractive purposes. In lieu of the latter, and particularly when multiple biopsy samples have been taken progressively over time, tumor samples at an earlier stage of evolutionary progression can be used as the “subtractor” material.

The target molecule(s), to which one or more of the aptamers bind, can be any protein or post-translationally modified protein, protein complex, carbohydrate, lipid, phospholipid, glycolipid, nucleic acid, or ribonucleoprotein associated with a cell. Particular target molecules include, but are not limited to, surface-expressed molecules, general and intracellular proteins, carbohydrates, lipid-related molecules, and nucleic acid molecules. Surface-expressed molecules include, but are not limited to: 1) integrins (such as, for example, integrin-β1); 2) melanocortin-1 receptor (MC1R); 3) other G-Protein coupled receptors (GPCRs); 4) immune cell markers (such as, for example, IgM, IgA, IgG, IgE (all isotypes), MHC Class I and Class II molecules, CD19, CD20, CD27, CD28, CTLA-4, and PD-1); 5) phosphatidylserine; 6) phosphatidylethanolamine; and 7) growth factor receptors (such as, for example, HER-2/neu and EGFR). General and intracellular proteins include, but are not limited to: 1) kinases; 2) enzymes; 3) transcription factors; 4) post-translationally modified proteins; 5) mutated proteins; and 6) protein complexes. Carbohydrates include, but are not limited to, complex carbohydrates appended to proteins (glycoproteins) or other molecules as carriers. Lipid-related molecules include, but are not limited to phospholipids and glycolipids. Nucleic acid molecules include, but are not limited to ribonucleoproteins and mRNA structural motifs.

In some embodiments, tumor cells are targeted by aptamers to allow selective cell killing by template assembly. In some embodiments, specific proteins or post-translationally modified proteins, protein complexes, carbohydrates, lipids, phospholipids, glycolipids, nucleic acids, and ribonucleoproteins can be targeted for aptamer binding and template presentation. The specific targets can be altered in some manner from the normal form such that they are restricted to cell lineage-specific, or any tumor cells, or altered in their normal cellular localization. Designated target molecules may be localized to cell surfaces, or found intracellularly, either within the cytoplasm or nucleus.

In some embodiments, where mutated tumor proteins have altered conformations, they provide useful targets for aptamer-mediated template presentation for the purposes of template assembly. Such conformational changes include, but are not limited to, misfolding and exposure of normally internalized residues, the induction of prion-like domains, and altered protein-protein interactions.

In some embodiments, tumor-specific protein target molecules are desired, and are potential targets for aptamer-based templated assembly. These include, but are not limited to, mutated oncogenes, growth factors, cell cycle regulators, and transcription factors.

In some embodiments, non-protein molecular tumor markers are desired, and are potential targets for aptamer-based template assembly. As a non-limiting example, phospholipids (including, but not limited to, phosphatidylserine and phosphatidylethanolamine) can be abnormally expressed on the exterior of tumor cells and tumor-associated vasculature in an “inside-out” manner.

In some embodiments, the target molecules within pathogenic cells may not necessarily be present initially, but become expressed as a consequence of specific prior or concurrent drug treatments. As one non-limiting example of tumor-specific marker expression induced by drugs, demethylating agents can induce endogenous retroviral sequences preferentially in colorectal cancer cells (Roulois et al., Cell, 2015, 162, 961-973). As another non-limiting example of this effect, abnormal surface phospholipid expression in tumors may in some cases be selectively enhanced by conventional cytotoxic drug treatments.

In some embodiments, abnormal clustering of surface molecules occurring during tumor cell development can be exploited as a target for aptamer-based template assembly. As a non-limiting example, it is known that both the composition of cell surface glycans and glycoproteins is markedly altered for certain tumor cells (Paszek et al., Nature, 2014, 511, 319-325), with resulting increased surface clustering of other molecules. As a result, important signaling proteins such as integrins attain increased spatial proximity on such tumor cell surfaces in comparison to matched normal tissue cells. Consequently, in some embodiments, aptamers can be developed against suitable surface-expressed integrins.

In some embodiments using aptamers as a means for positioning surface templates on target cells, surface immunoglobulins may be employed for such purposes. As a non-limiting example, the BJAB tumor is an IgM secreting B cell line that also expresses its monoclonal IgM on the cell surface. An aptamer known to bind to the BJAB cell line has been described (Zumrut et al. 2017), and may be used to serve the dual purpose of binding specifically to the surface immunoglobulin molecule and also serving as a template for haplomer assembly of the trastuzumab mimotope. In some embodiments, the BJAB aptamer sequence is CACTGGGTGG GGTTAGCGGGCGATTTAGGGATCTTGAGTGGTGGA (SEQ ID NO:115). In some embodiments, the BJAB aptamer sequence with appended 3′ sequence for templating of haplomers is CACTGGGTGGGGTTAGCGGGCGATTTAGGGATCTTGAGTGGTGTCAAAA GCCAAAAAGCCACTGTGTCCTGAAGAAAGCAAAGACATCTGGACAAAAAGC (SEQ ID NO:116).

An alternative strategy for such surface template positioning is by means of a template conjugate with a specific ligand for a surface receptor present on target cells of interest. One such example is the melanocortin-1 (MC1R) receptor, which has a well-characterized interaction with alpha-melanocyte stimulating hormone, and certain known analogs of it. Since such MC1R ligands are short peptides lacking cysteine residues, they are amenable to conjugation with desired template nucleic acids via maleimide chemistry.

Briefly, Left and Right components of binary aptamers can be directed towards short contiguous peptides within known target proteins whose structure is available, or whose structure has high conformational flexibility, or whose structure is intrinsically disordered. A non-limiting example of this is the N-terminal extracellular domain of MC1R, which is comprised of 36 amino acid residues and widely expressed on normal melanocytes and melanoma cells. Pentapeptide sequences within this tract can serve as independent aptamer targets, with best candidates bearing a preponderance of charged or hydrophilic residues. The chosen sites, referred to herein as “epitopes” (e.g., SQRRL (SEQ ID NO:117) and QTGAR (SEQ ID NO:118) in order from the N-terminus), also bear one or more arginine residues, which is advantageous for aptamer targeting owing to the positive charge carried by the arginine side-chain at neutral pH (Geiger et al., Nucleic Acids Res. 1996, 6, 1029-1036). While numerous proteins bear either of these pentapeptide sequences, no known proteins (in addition to MC1R) with both sequences exist in current databases. Co-binding experiments with both combinations of L- and R-aptamer subpopulations binding these pentapeptides have been carried out.

In some embodiments, L- and R-aptamer subpopulations binding separately to SQRRL (SEQ ID NO:117) and QTGAR (SEQ ID NO:118) can be selected by standard procedures. For example, each combination of L- and R-aptamers against the two pentapeptides can be subjected to the co-binding process on intact melanoma cells previously shown to express MC1R. Specific co-binding of L/R aptamers under such circumstances occurs on MC1R N-termini but not elsewhere. Binary aptamer binding to MC1R allows template assembly for split epitopes directed at the melanocytic cell lineage, including melanoma cells.

In a variation of this embodiment, L- and R-aptamers can be selected for D-isomers of the SQRRL (SEQ ID NO:117) and QTGAR (SEQ ID NO:118) sequences. This affords the opportunity to subsequently synthesize L-aptamers (spiegelmers; from the derived sequences of the selected normal aptamers with D-ribose chirality) which recognize the opposite chirality of the original target (normal L-amino acids).

In some embodiments, the accessible short amino acid tracts can be hydrophobic residues specifically exposed on tumor-related proteins through aberrant folding, associated with induction of the Unfolded Protein Response.

In some embodiments, an aptamer, whether as a constituent of a binary pair or as a singlet, binds to surface anionic phospholipids, including, but not limited to phosphatidylserine, phosphatidylethanolamine and phosphatidylinositol. In some embodiments, the selection for aptamers binding anionic targets is augmented through the provision of cofactors bearing a positive charge at neutral pH. These include, but are not limited to, small amines such as putrescine, spermine, and spermidine.

In some embodiments, the cell surface target is the human integrin-β1 extracellular domain.

In some embodiments, the target molecule is an antibody or cell surface protein. In some embodiments, the antibody is IgM. In some embodiments, the cell surface protein is melanocortin-1 receptor (MC1R).

The recognition molecule can be any molecule that recognizes the assembled epitope. In some embodiments, the recognition molecule is an antibody, or a fragment thereof including, but are not limited to, Fab, F(ab′)₂, monospecific Fab₂, bispecific Fab₂, trispecific Fab₃, scFv, scFv-FC, bispecific diabody, trispecific triabody, minibody, nanobody, IgNAR, V-NAR, hcIgG, and VhH proteins. Also included are structures with artificial complementarity-determining regions such as, for example, ankyrin repeat proteins, affimers, avimers, and nucleic acid aptamers of any composition.

In some embodiments, when the epitope is within erb-B2, the therapeutic agent is trastuzumab (HERCEPTIN®). In some embodiments, when the epitope is within the glycoprotein F of respiratory syncytial virus (RSV), the therapeutic agent is palivizumab (SYNAGIS®). In some embodiments, when the epitope is within the glycoprotein F of RSV, the therapeutic agent is motavizumab (NUMAX®). In some embodiments, when the epitope is within EGFR, the therapeutic agent is panitumumab. In some embodiments, when the epitope is within the Vi antigen of Salmonella enterica, the therapeutic agent is ATVi. In some embodiments, when the epitope is within the H5N1 Influenza Virus, the therapeutic agent is AVFluIgG01. In some embodiments, when the epitope is within CD147, the therapeutic agent is metuximab (LICARTIN®). In some embodiments, when the epitope is within Schistosoma mansoni, the therapeutic agent is 152-66-9b.

The present disclosure also provides methods of delivering at least one aptamer to a pathogenic cell. In some embodiments, the method comprises: administering a therapeutically effective amount of any one or more aptamers and a corresponding epitope haplomer pair described herein to the pathogenic cell. In some embodiments, at least one epitope in the pathogenic cell is produced. In some embodiments, the aptamer is administered separately from one or both epitope haplomers. In some embodiments, at least one of programmed cell death of the pathogenic cell, apoptosis of the pathogenic cell, non-specific or programmed necrosis of the pathogenic cell, lysis of the pathogenic cell, and growth inhibition of the pathogenic cell is produced. In some embodiments, the pathogenic cell is selected from the group consisting of a virus infected cell, a tumor or cancer cell, a cell infected with a microbe, and a cell that produces a disease-inducing or disease modulating molecule that may cause inflammation, allergy or autoimmune pathology.

In some embodiments, the template assembly process can be effectively exploited for in vitro cellular selection processes, or cellular diagnostics. This is particularly applicable to binary approaches where it is more facile to assemble a binary on a target molecule, as with L-DNA tagged binaries or binary allosteric aptamers. This can be amenable to in vitro applications, particularly for directed identification and selection of rare cellular subsets.

In some embodiments for diagnostic or research purposes, target cell subpopulations in vitro can be labeled for fluorescence-based cell sorting, by means of, for example, binary fluorescent aptamer binding and effector partial moiety templating. The fluorescent moieties can be carried by either or both aptamers, or through the agency of the reaction between haplomers.

In some embodiments, for diagnostic, therapeutic, or research purposes, target cell subpopulations in vitro can be removed by binary aptamers which deliver a template assembly-mediated killing signal. One example of this method is a negative selection for subpopulations not recognized by the specific binary aptamers used in such circumstances.

In some embodiments, for diagnostic, therapeutic, or research purposes, specific cell subpopulations in vitro can be targeted by binary aptamers which direct the templated assembly-mediated production of a positively selectable marker.

In some embodiments, the selectable marker is comprised of, but not limited to, fluorescent moieties, peptides or other molecular structures for which antibodies are available, or assembled affinity tags for available protein-ligand systems.

The present disclosure also provides the foregoing methods further comprising administering to the human a therapeutic agent that selectively binds to the assembled epitope. Suitable antibodies include, but are not limited to trastuzumab, palivizumab, and motavizumab.

Numerous advantages exist for aptamer-mediated adaptive templating compared to conventional templated assembly. For example, aptamers greatly expand the range of targetable molecules: to proteins, peptides, carbohydrates, particular amphiphilic lipids (e.g., phospholipids), and nucleic acid structures not otherwise targetable by conventional template assembly (such as highly folded RNA secondary structures). Aptamers also allow template assembly to be performed on cell surfaces. Cell-surface templating circumvents many delivery issues, since cell penetration is not required.

Numerous advantages also exist for aptamer-mediated adaptive templating compared to antibody-based alternatives. Conversion of diverse cell surface targets into a common target structure for immune recognition is possible with aptamers. For example, aptamer-mediated recognition of a target cell surface structure allows template assembly of a traceless peptide recognized by a previously developed antibody or a CAR-T system. Also, aptamer-mediated recognition of a target cell surface structure allows templated assembly of a click-ligated peptidomimetic recognized by a previously developed antibody or a CAR-T system. In both of these examples, both the aptamer templating region and the complementary haplomers bearing the reactive half-epitopes are modular, and if L-DNA tags are used, the system can involve bioorthogonal hybridization.

In addition, where target structures are previously known, development of antibody-drug complexes or CAR-T systems is complex and expensive. In contrast, following isolation of a specific recognition aptamer, an adaptive templating system is “ready to use,” and exploits pre-existing template assembly technology.

Where target structures are newly defined, it is much quicker and cheaper to develop a new aptamer that combines target specificity and template assembly template than a corresponding antibody.

Where target structures are unknown, aptamer libraries can be used for subtractive approaches to detect novel surface structures on tumor cells absent from normal cells, or novel structures on drug-treated tumor cells vs. untreated tumor cells. This is impractical or much more difficult in the case of antibody-based technologies.

For all cases where antibodies might be used instead of aptamers, the relatively small sizes of aptamers provides a distinct advantage for tumor cell access in tumor microenvironments. Also, it is much more probable that aptamers can be efficiently transfected within cells (for binding intracellular targets) than large protein molecules such as antibodies.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Demonstration of Co-Binding Selection on a Solid-Phase Target, and Sequence Confirmation of Binary Aptamer Candidates after Co-Binding Selection

After 4 cycles of separate selection of Left- and Right-aptamer libraries, eluted subpopulations were incubated with the biotinylated immunoglobulin Fab target (against BRD7 protein; ThermoFisher), and then rendered solid-phase by binding to streptavidin-magnetic beads. After 3 washes with 0.5 ml PBSM and 1 wash with 1×T4 DNA ligase buffer (NEB, containing 1 mM ATP), preparations were subdivided into two equal halves and subjected to +/−annealing with the DNA splint (5′-TCCAGATGTCTTTGCTTTCTTCAGGACACAG (SEQ ID NO:119); 100 μl, 1 pmol/μl), by heating for 5 minutes at 37° C., and then holding for 1 hour at room temperature. Following this, the preparations were again washed 2 times with ligase buffer. Tubes were then split once more into two equal parts, and treated with +/−T4 DNA ligase. Small samples (1 μl) of these reactions were then amplified with primers Trz.F/Trz.R, and tested on 10% non-denaturing acrylamide gels. This showed that a strong product of binary size was observed with the 4th-cycle material, only via the agency of ligase, and only in the presence of the splint. Significantly, a comparable strong product band was not seen from the original (unselected) aptamer Left- and Right-libraries. This demonstrates that the cycles of binding, washing, and amplification had significantly enriched for Fab-selective binders over the original unselected populations. Sequencing revealed that the amplified product from the co-binding test for the 4th cycle material showed perfect fusion of the Left- and Right-aptamer components, as joined via the splint oligonucleotide.

In particular, successful co-binding after 4 cycles of Fab selection has been demonstrated, and sequence analysis of a co-binding experiment binary aptamer product. Cobind-01, arbitrary example of cloned product from EL4 Left- and Right-aptamer populations subjected to co-binding process. Bold sequences are 40-mer tracts deriving from randomized sequence in the original aptamer libraries. Boxed sequences are primer sites. No fill is primer Trz.F. Light gray fill is primer AptInt.R (antisense in this orientation). Speckled fill is primer AptInt.F. No fill, dark lines is primer Trz.R (antisense in this orientation). This specific sequence example can be compared with the general structure of binary aptamers.

### Example 2: Singlet Aptamer Analysis and Binary Aptamer Generation (Co-Binding Process) after 10 Cycles of Selection on Fab Fragments

After 10 cycles of separate selection for singlet Left- and Right-aptamer subpopulations on biotinylated Fab target, the resulting subpopulations were cloned and sequenced. Here (in contrast to results from the 4th cycle), multiple recurrences of a specific aptamer sequence were observed. From 14 sequenced specific singlets (7 each from Left- and Right-clone populations), 3 recurrences of a specific Right-aptamer (designated as 288/10AptR1) were found.

Left- and Right-10th cycle aptamer subpopulations from the Fab target were then subjected to the co-binding procedure on the Fab target. The amplified binary products were then sequenced and characterized. It was found that the Right-aptamer clone 228/0AptR1, previously observed as a recurrent singlet clone, was also found independently in 5 independent binary clones. Notably, in one of these binary clones (10CB-10), the Left-aptamer component (229/10AptL3) had been previously independently isolated and sequenced from the 10th cycle Left-aptamer subpopulation. The recurrence of identical sequences in both the singlet and binary aptamer subpopulations that had been selected for Fab binding was consistent with the expected reduction in the subpopulation size towards a set of aptamers with useful Fab-binding affinity.

In particular, a 10th cycle analyses of aptamers binding biotinylated Fab was performed. Recurring singlet aptamer clone 228 (10AptR1) is demonstrated. A co-binding test of 10th cycle L- and R-aptamers to biotinylated Fab was performed. A product was observed for the selected (10th cycle) aptamer pairs, but no such product were seen with primary (unselected) aptamers at this level of sensitivity. The cobinding process was equivalent to that used for 4th-cycle aptamers.

In particular, direct binding of specific 10th-cycle aptamers (10AprR1, 10AptL3) to bFab (Direct Binding Assay using streptavidin magnetic beads) was demonstrated. Aptamers were incubated with bFab (2.5-fold molar excess), then bound to SAMBs. Supernatants were taken, and the SAMBs washed 3 times. Bound material was eluted with 0.1 M NaOH, and then precipitated, washed, dried, and reconstituted before loading samples on a denaturing acrylamide gel. “No biotinylated Fab” (bFab) indicates that no bFab was present during initial incubation, but aptamers were then treated with SAMBs in common with the (+) bFab tubes.

### Example 3: Direct Demonstration of Binding of Specific 10th-Generation Singlet Aptamers Towards the Fab Target

To assess binding of 10^{th}-cycle singlet aptamers to the selective agent (the Fab target), direct binding assays were carried out. Here, single-stranded aptamer preparations (self-annealed as usual) were incubated with or without biotinylated Fab fragments in PBSM, followed by adsorption onto streptavidin magnetic beads. After this incubation period, the beads were magnetically separated, and the supernatants retained. After 3 washes of the beads, the bound material was eluted by means of 2×20 second incubations with 100 μl of 0.1 M NaOH, with the eluates immediately precipitated with 0.3 M sodium acetate and 3 volumes of ethanol Pellets were washed with 70% ethanol, and dried. After reconstitution in 5 μl of TE, 1 μl samples were denatured in formamide and run on 10% urea (denaturing) gels. Results of such an experiment with the candidate singlet aptamers 228/10AptR1 and 229/10AptL3 and a specific arbitrary unselected control Right-aptamer (#136; sequence corresponds to: GCAAAGACATCTGGACA CGCCACTTATAGTCTACGTGAAGCACTGCGCTGGAACAGCCTAAAAAAGGAGAAGG AGACTTAGAGGC (SEQ ID NO:120); where underlining indicates the 40-mer aptamer tract; remaining sequences are primer sites) demonstrate that the supernatants from the 228/10AptR1 and 229/10AptL3 binding (but not #136) were depleted only in the presence of biotinylated Fab. Moreover, the eluted material from biotinylated Fab on the streptavidin magnetic beads was highly enriched for aptamer bands only for 228/10AptR1 and 229/10AptL3. These results strongly suggest that the selected aptamers 228/10AptR1 and 229/10AptL3 showed significant and specific interaction with the Fab target.

In particular, a representative binary clone 10CB-01 was obtained from 10th cycle co-binding experiment. The Left-aptamer component of this binary (229/10AptL3) was previously independently isolated directly from the Left-singlet subpopulation; the Right-aptamer component (228/10AptR1) was previously independently isolated directly from the Left-singlet subpopulation.

### Example 4: Direct Demonstration of Binding of Specific 10th-Generation Binary Aptamers Towards the Fab Target

Despite successful in situ assembly of binary aptamers upon the target to which they were bound (as in Example 1), it could not be assumed that binary aptamers formed in solution would be capable of binding the same targets. This was assessed using the same aptamers as used in direct binding tests (Example 3; 228/10AptR1 and 229/10AptL3), but where the aptamers were initially ligated together by means of the same splint oligonucleotide as used in Example 1. Under the same experimental conditions as in Example 3, when equivalent samples were run on a denaturing gel, a bound band corresponding to the binary product was seen, as well as the corresponding splint oligonucleotide as expected. One of the component singlet aptamers (228/10AptR1) was used as a control, and a bound band was observed, as previously shown (see, Example 6). No binding of the binary product to streptavidin beads alone was observed, indicating the requirement for Fab binding. In this case, an additional control was used with a known aptamer with binding affinity directly for streptavidin, and here a bound band was seen independently of the presence of Fab as predicted.

### Example 5: Co-Binding Tests on IgG1 Antibody

Although the target for selection in the above examples was biotinylated Fab, it was desirable that the derived aptamers from this process could also recognize intact immunoglobulin of the same isotype (murine gamma 1). This example serves as a generic demonstration of the use of a smaller component of a larger molecule or molecular complex to initially identify separate Left- and Right-binding aptamers, and then use these initial subpopulations to identify binary aptamers recognizing the larger desired target.

Left- and Right-aptamer populations from the 10th cycle of selection on biotinylated Fab were used for co-binding testing on intact murine IgG1 (Santa Cruz Laboratories). This was carried out in an equivalent manner as for previous co-binding tests on biotinylated Fab, but where the IgG1 replaced the Fab (in the same molar amounts), and the IgG1 itself was adsorbed to the sold phase by binding to Protein G magnetic beads (New England Biolabs). Following splint-mediated ligation, washing, and elution (as for Example 2), products were amplified (25 cycles) with primers defining ligated binary aptamers, and analyzed on a non-denaturing acrylamide gel. Results showed that the selected Left- and Right-aptamer populations gave rise to a binary product band, which was only produced when both the splint oligonucleotide and ligase were present. No such easily detectable bands were observed from the primary (unselected) aptamer libraries.

Co-binding on IgG1 target of 10th-cycle Fab-selected aptamers was performed. The method for co-binding was equivalent to that described herein, except that IgG1 was the target rather than biotinylated Fab, and selection on solid-phase was accomplished by binding the IgG1 to Protein G-magnetic beads.

### Example 6: Effector Oligonucleotide Templating on Solid-Phase Templates

For aptamers to be useful for template assembly, they should display accessible sequences of sufficient length to act as templates for effector partials. The ability of the designated aptamer sequences to act as templates was initially assessed by means of corresponding desthiobiotinylated oligonucleotide sequences rendered solid-phase by capture on streptavidin magnetic beads. As a convenient model for template assembly reactivity with traceless Staudinger chemistry, oligonucleotides modified with Inverse Electron-Demand Diels-Alder (IEDDA) chemical reactants were employed. In order to do this, oligonucleotides with 5′ or 3′ amino-modifications were reacted with N-hydroxysuccinimide-activated trans-cyclooctene (TCO) ester or corresponding methyltetrazine (MTZ) ester for 4 hours at room temperature in phosphate-buffered saline. After this, unreacted esters were removed by desalting columns (BioRad). The resulting oligonucleotide adducts could be distinguished from unreacted control corresponding oligonucleotides on denaturing gels via clear-cut mobility differences.

Although test oligonucleotides annealed to target templates and joined via IEDDA click chemistry cannot be directly amplified, the product can be amplified by inverse PCR if the opposite ends of the oligonucleotide pair are conventionally ligated. To effect this, the test template-complementary oligonucleotides (207 and 208) were equipped with mutually compatible restriction sites. Prior to use in templating tests, the TCO-modified 207 and MTZ-modified 208 oligonucleotides were annealed with 28-mer oligonucleotides complementary to their 3′ and 5′ ends, respectively. (Oligonucleotide complementary to 3′ end of 207: TGTAGGA CTCTAGATCGGAAGTTGTAGC; SEQ ID NO:121; Oligonucleotide complementary to 5′ end of 208: CTCGAAGGCTACGTGCTAGCGCATACAT; SEQ ID NO:122). Following this, the partially-duplexed TCO-modified 207 and MTZ-modified 208 oligonucleotides were digested with Xba I and Nhe I, respectively. When these oligonucleotides mutually anneal to a template where their complementary sites are adjacent, the digested ends are in close proximity to each other and can be efficiently ligated by T4 ligase. The ligated product is amplifiable by PCR, inverse with respect to the original 5′ and 3′ ends of the oligonucleotides.

TCO-modified 207 oligonucleotide bearing the above Xba I site 5′ overhang was annealed with desthiobiotinylated target (aptamer-junction) oligonucleotide, and then the material bound to streptavidin magnetic beads in phosphate-buffered saline with 1 mM MgCl₂ (PBSM). After three washes with PBSM, excess MTZ-modified 208 oligonucleotide was added and incubated 5 minutes at 37° C. and 1 hour at room temperature, followed by three more washes. Following this, the solid-phase magnetic bead preparations were washed twice in ×1 T4 DNA ligase buffer with 1 mM ATP (NEB), and split into two portions, with and without 400 units T4 DNA ligase. After 2 hours at room temperature, the preparations were washed in PBSM, and bound material was then eluted from the streptavidin magnetic beads by incubation with 100 μM D-biotin (Sigma). Samples were then run on a 10% denaturing acrylamide gel.

Results showed that IEDDA click product between the TCO-modified 207 and MTZ-modified 208 oligonucleotides formed on the solid-phase template. This band was size-shifted by ligation of the restriction site ends, corresponding to an expected circularization process. Unmodified control oligonucleotides showed no band with the IEDDA product mobilities, but did show a ligation-specific band corresponding to restriction end joining.

Solid-phase oligonucleotide-based templating using sequences present in aptamers was performed. Template and oligonucleotide sequences are as described herein. It was subsequently shown with the same eluted material that PCR product formation (inverse with respect to the IEDDA joining site) was possible, but only after in situ ligation of the restriction ends as expected. This demonstrated that inverse PCR is a suitable read-out for in situ templating of model templated assembly reactions.

### Example 7: Aptamer-Mediated Templating of Effector Oligonucleotides on Target

Following demonstration of templating on solid-phase oligonucleotides corresponding to the binary aptamer templating regions, it was shown that templating can be effected on aptamer templates themselves, while bound to specific targets in situ. Both L- and R-aptamers selected for binding biotinylated anti-BRD7 Fab (bFab) and arbitrary unselected control L- and R-aptamers were separately self-annealed and incubated in appropriate combinations (140 pmol of each aptamer; 25 μl final volumes) with and without 35 pmol of the bFab target (see, Table 1). After 1 hour at room temperature, the preparations were treated with 100 μl streptavidin magnetic beads for 30 minutes at room temperature with shaking (where beads were initially magnetically separated from the storage medium, washed twice with 1 ml of PBSM, and resuspended in the original volume of PBSM prior to use). Following this, beads were magnetically separated and washed once with 0.5 ml PBSM, and twice with 100 μl of ×1 SplintR® ligase buffer (New England Biolabs), and resuspended in 50 μl of SplintR® ligase buffer (with ATP) also containing 60 units murine ribonuclease inhibitor (NEB). Subsequently, 140 pmol (1.4 μl) of an RNA oligonucleotide was added, corresponding to the complement to the L/R inter-aptamer region, with the sequence: UCCAGAUGUCUUUGCUUUCUUCAGGA CACAG (SEQ ID NO:123). The preparations were annealed for 5 minutes at 37° C., and then at 30° C. for 1 hour, prior to the addition of 25 units of SplintR® ligase (New England Biolabs, a Chlorella ligase with high nick-sealing ability for DNA strands on RNA templates). After 1 hour at room temperature, the magnetic beads with the bound bFab/aptamer- RNA duplexes were washed once with 100 μl of RNase H buffer (New England Biolabs), and then resuspended in 50 μl of the same buffer with 5 units RNase H (New England Biolabs) for 10 minutes at 37° C. and 20 minutes room temperature. After washing once with 0.5 ml PBSM, samples were resuspended in 50 μl of PBSM. Then 105 pmol (5.3 μl; 3-fold molar excess over initial amount of bFab) of methyltetrazine-3′-modified oligonucleotide 208 (as in Example 6) for 30 minutes at room temperature, followed by washing with 0.5 ml PBSM and resuspension in 50 μl of PBSM. Subsequently, 105 pmol (5.3 μl; 3-fold molar excess over initial amount of bFab) of trans-cyclooctene-5′-modified oligonucleotide 207 (see, Example 6) were added, also for 30 minutes at room temperature. Preparations were then washed with 0.5 ml PBSM and bound DNA was eluted with two treatments with 100 μl of 0.1 M NaOH/5 mM EDTA for 20 seconds at room temperature (pooling of magnetically-separated supernatants), followed by immediate precipitation at −20° C. (for 30 minutes) with 0.3 M NaOAc, 20 μg glycogen, and 3 volumes of 100% ethanol. Preparations were washed with 1 ml of 70% ethanol, dried, and re-dissolved in 4.0 μl TE. Samples (1.0 μl) were run on 15% urea denaturing gels.

TABLE 1
Aptamer templating experiment (Example 7)

| | +/−L- | +/−R- | | | |
|---|---|---|---|---|---|
| | Aptamer | Aptamer | +/−bFab- | +/−RNA | +/−RNase |
| Reaction no. | (code) | (code) | BRD7 | Splint | H |
| 1 | +(229) | +(228) | + | + | + |
| 2 | +(229) | +(228) | + | − | − |
| 3 | +(229) | +(228) | − | + | + |
| 4 | +(229) | +(228) | + | + | + |
| 5 | +(229) | +(138) | + | + | + |
| 6 | +(139) | +(228) | + | + | + |
| 7 | − | − | + | + | + |
| 8 | − | − | − | + | + |

140 pmol of each aptamer was initially self-annealed, and then incubated in the reaction tubes, with or without 35 pmol biotinylated anti-BDR7 Fab (bFab; 40-fold aptamer excess). 229, 228: specific L- and R-bFab-binding aptamers; 139, 138: arbitrary L- and R-aptamer sequences not selected for bFab binding, shown in bold.

Gel analysis showed that reaction between the model click-labeled oligonucleotides was present on both specific aptamers as templates bound to bFab. However, in this case, splint-mediated ligation of L-(229) and R-(228) aptamers was unnecessary, as product was observed when splint/ligase was omitted. That binary aptamers via splint-ligation were formed was shown with primers specific for both binary and (as an example of a singlet aptamer) R-aptamer forms. Singlet aptamers with the R-primers were only seen for preparations with the bFab-binding R-aptamer #228 as expected. And binary products of #228 with its partner #229 were only observed when splint and ligase were applied.

Templating of model IEDDA click reactions by aptamer templates while bound to bFab target in situ was carried out. Aptamers #229 and #228 were originally selected as proximal binaries on bFab target (p-228 denotes the presence of a 5′ phosphate group to enable ligation with its partner aptamer via the RNA splint); Aptamers #139 and #138 are known non-binders.

PCR testing of binding and binary formation of aptamers bound to bFab in situ was carried out. All preparations with #228 (known R-aptamer bFab binder) show good R-singlet bands. However, only the duplicate preps with splint+ligase showed the presence of the binary band. Unligated #228/#229 showed a strong R-singlet band (showing bFab binding) but no binary band.

While aptamer-mediated proximity alone could promote the templated assembly of click-labeled oligonucleotides, other templating applications may benefit from the contiguous longer template afforded by binary L-R aptamer pairs. Thus, pre-formed binary #229-#228 aptamer was prepared by annealing both aptamer strands with the above RNA template at high concentrations, and then removing the template with RNase H. To remove remaining singlet strands, the 170-base binary strand was purified on an agarose gel. Subsequently, it was demonstrated that the assembled binary aptamer still bound to the target biotinylated Fab, and provided accessible template for the 207-208 labeled oligonucleotide click reaction.

The formation and testing of binary aptamers in situ on bFab target was carried out. RNA-splint-mediated formation of binary aptamers between singlet L- and R-aptamers #229 and #228 respectively, was observed in solution at high concentration. The purified sample (1.5% agarose) of 170 bp #229-#228 binary with the splint was removed by RNase H (denaturing acrylamide gel). The formation of model click product on binary aptamers bound to specific bFab target was observed.

### Example 8: Formation of Accessible Template in Binary Aptamers by Means of a Short Stem-Loop Bridge

Although formation of binary aptamers can be effected in situ by means of a removable RNA splint (see, Example 6), an alternate method was developed where no ligation is necessary. Here, short complementary sequences were appended onto the 3′ and 5′ ends of L- and R-aptamers respectively, where they bind in proximity to a common target. As a consequence of this, the mutually complementary modified ends of the aptamers form a short stem-loop bridge. It is known that stem-loops can function for templating for template assembly purposes, demonstrated using model click oligonucleotides.

Alternately, binaries can be assembled via stem-loop hybridization in solution. The aptamer pair #229 (L) and #228 (R) targeting the biotinylated Fab-BRD7 protein were synthesized with mutually complementary 10-base 3′ and 5′ ends respectively. Although the appended segment sequence is arbitrary, here a G/C sequence was used for maximum duplex stability. A short stem sequence is desirable to minimize the chance that the appended segment will interfere with aptamer function, and sequences complementary to the 40-base aptamer region are thus excluded. Nevertheless, the successful addition of an appended segment compatible with aptamer function should still be tested empirically. A functional test for the appended aptamers was performed. The #229 aptamer binding for the biotinylated Fab was reduced somewhat for the stem-loop tag, but less so for a control tag with the same base composition but scrambled sequence. The #228 aptamer was functionally little affected by the presence of the appended tag.

Alternate in situ formation of template from a proximal binary aptamer pair by means of a short step-loop bridge was performed. It is known that stem-loop structures in general can be used for model click oligonucleotide templated reactions.

Testing the effect of aptamer extensions on ability to bind bFab-BRD7 was carried out. 140 pmol of self-annealed aptamers were incubated with 35 pmol bFab (25 μl final volume) at room temperature for 3.5 hours, then adsorbed onto 50 μl streptavidin magnetic beads in PBSM for 1 hour at room temperature. Supernatants were then magnetically removed, and the beads washed twice with 0.5 ml of PBSM. Bound material was eluted with 2×100 μl of 0.1 M NaOH/5 mM EDTA, precipitated with 20 μg of glycogen/0.3 M NaOAc, 3 volumes of ethanol, washed once with 1 ml of 70% ethanol, dried, and redissolved in 20 μl of TE. One μl samples were run directly on an 8 M urea denaturing gel.

The extended aptamers were then assessed for their abilities to act as templates for model click reactions. Extended aptamers 229-3′-Ext1 and 228-5′-Ext1 were annealed together (3 minutes at 80° C., then 5 minutes at 0° C.), to allow aptamer self-annealing, and also inter-aptamer hybridization via the mutually complementary 10-base extensions. Control aptamers #229, #228, and 136-5′-Ext1 were separately self-annealed in the same manner Aptamer preparations were incubated with bFab target, washed, and bond material eluted with sodium hydroxide. After precipitation, washing, and drying, eluted material was reconstituted in 10 μl TE, with 1 μl samples run on a denaturing urea gel. Results showed that proximal aptamers alone (spatially close but lacking a binary join) could enhance templated click reactivity. Likewise, control aptamers with 3′ and 5′ extensions without mutual complementarity were capable of similar click activity promotion. However, not only could the stem-loop linked binary preparations still support click activity, but the product level was increased relative to the control. Whether this is a consequence of improved target binding itself, or enhanced templating, the end result still indicates improved templating for template assembly.

Testing of the complementary-end binary stem-loop aptamer approach, with the extended aptamers 229-3′-Ext1 and 228-5′-Ext1 was carried out. These aptamers were self-annealed and co-annealed simultaneously in solution to form the stem-loop linked binary. Unextended aptamer controls were self-annealed separately as usual. The principles demonstrated within this example are analogous to, but distinct from, the L-DNA tagging procedure described above.

### Example 9: Affinity Measurements for Specific Aptamers

Aptamer affinities for defined targets are measurable by QPCR-based methods, in conjunction with a process for distinguishing bound from unbound aptamer over a range of target concentrations. This was applied to the aptamer #228, which was selected for bFab binding as a singlet, and binary binding with its partner #229. To construct a binding curve, dilutions of the bFab target (from 700 nM downwards in 2-fold dilutions) were incubated with a constant concentration of self-annealed #228 (10 nM) overnight (50 μl final volumes in PBSM) such that equilibrium conditions were attained. The preparations were then incubated with 75 μl of streptavidin magnetic beads (in molar excess over the highest concentration of bFab) for 1 hour at room temperature with shaking. Beads were then magnetically separated from supernatants, with each tube subjected to three washings with 0.5 ml of PBSM (original supernatants and washings were combined to give a total unbound fraction). Bound material was subsequently eluted from the beads by 2×20 second incubations with 0.1 M NaOH/5 mM EDTA, pooling the magnetically-separated eluate supernatants into a single tube. These were immediately precipitated with 20 μg glycogen/0.3 M NaOAc/3 volumes of ethanol (for 30 minutes at −20° C. incubation), followed by washing with 1.0 ml of 70% ethanol, drying, and reconstitution in 50 μl of PBSM. Samples of all preparations (1.0 μl) were then analyzed in triplicate in 96-well plates by QPCR, by means of a Bio-Rad CFX96 Touch instrument, with a cycle of 95° C. for 30 seconds; 40×(5 seconds at 95° C., and 30 seconds at 60° C.), in 20 μl volumes. Reaction mixes used ×1 BioRad iTaq PCR mixes and 6 pmol each of R-aptamer-specific primers. (Forward R-primer: GCAAAGACATCTGGACACGC (SEQ ID NO:124); Reverse R-primer: GCCTCTAAGTCTCCTTCTCCT (SEQ ID NO:125)). Wells were analyzed during cycling for real-time SYBR-green fluorescence, and CT values assigned. All runs included a standard curve of serial dilutions of #228 aptamer. Replicate results were averaged and bound and unbound CT values were derived for each data point, allowing calculation of total bound fractions. From a plot of these bound fraction values vs. corresponding [bFab], a non-linear regression curve could be derived. In turn, a K_{d} estimate (of about 11 nM) could be obtained from the equation for the experimental curve where K_{d} corresponds to fraction bound=0.5 (Jing et al., Anal. Chim Acta, 2011, 686, 9-18).

A binding curve for aptamer #228 and biotinylated Fab-BRD7 was produced. Non-linear regression curve equation is y=0.11791n(x)+0.2181.

### Example 10: Aptamer-Mediated Surface Assembly of a T Cell HLA-A2 Restricted Epitope

Aptamers can be used to adapt a wide variety of surface structures as templates for the template assembly process, and this adaptation process can include multiple recognition molecules, in a “sandwich” type of arrangement. This example discloses the use of a biotinylated target molecule towards which a binary aptamer pair is directed. It also uses a biotinylated primary recognition molecule binding to a desired and pre-defined cell-surface marker, and a multivalent biotin-binding bridging molecule.

In this instance, the target molecule is a biotinylated anti-BRD7 Fab (see, Examples 1-4 and 7-9), the primary recognition molecule is a biotinylated anti-IgM (BD-Pharmingen), and the bridging molecule is a streptavidin-phycoerythrin conjugate (SA-PE; Fitzgerald Industries). Streptavidin alone (Sigma-Aldrich) can also be used in lieu of the phycoerythrin conjugate. Cells of interest (10⁶) expressing surface IgM (EBV-transformed lymphoblastoid cell lines) are harvested, washed with ×1 PBS, and treated for 1 hour at room temperature with biotinylated anti-IgM at a suitable concentration (as recommended by the manufacturer). Following 3×PBS washes, 100 pmol of SA-PE previously complexed in an appropriate molar ratio with biotinylated anti-BRD7 Fab (bFab) is incubated with the primed cell suspension for 1 hour at room temperature, with occasional resuspension of the cells. The pre-assembled SA-PE complex is created in the following manner: 50 pmol SA-PE is incubated with 50-100 pmol bFab for 1 hour at room temperature, in 1×PBSM. Since SA is tetravalent, this ensures that all bFab is bound without saturating the available SA biotin-binding sites. The cells are then washed twice with PBSM, and resuspended in 0.5 ml of PBSM. Pre-annealed aptamers 229-3′-Ext1 and 228-5′-Ext (forming a binary via a stem-loop bridge, as in Example 8; 100 pmol) are added to the primed cells for 1 hour at room temperature, and washed twice with 1.0 ml of PBSM.

The success of the formation of the multi-layered sandwich is assayed at two levels. The presence of the target surface antigen (IgM) is demonstrated by subjecting complexed cell samples (primary anti-IgM antibody/SA-PE/bFab/binary aptamer) to flow analysis for fluorescence in the PE channel, in comparison with control cells treated in an identical manner except for the exclusion of the primary anti-IgM antibody. Aptamer binding is demonstrated with a bilabeled fluorescent splint oligonucleotide (as for the DNA splint in Example 1 (DNA splint (5′-TCCAGATGTCTTTGCTTTCTTCAGGACACAG; SEQ ID NO:119) except for its modification at both 5′ and 3′ ends with fluorescent FAM moieties). The fluorescent splint (100 pmol) is incubated in PBSM with fully complexed cells and controls identical except for exclusion of the bFab for 1 hour at room temperature, and then cells are washed three times with 0.5 ml of cold PBSM before being subjected to flow analysis with the fluorescein channel.

Preparations passing these tests can be used for assembly of a Melan A/MART epitope (ELAGIGILTV; SEQ ID NO:126) presented by HLA-A2, since the binary aptamer templating regions in this system are designed to hybridize with the haplomer Human-Papillomavirus-derived sequences described in the application PCT International Publication WO 14/197547.

Complexes on EBV-transformed HLA-A2+ cells expressing surface IgM recognized by the primary biotinylated antibody are equipped with the binary stem-loop aptamers as detailed above in this Example. Following washing as above, preparations are incubated with both haplomers recognizing the binary aptamer templating region for 1 hour at room temperature, and bearing MART epitope half-peptides. During this incubation, the haplomers hybridize to the aptamer surface template in proximity to each other, allowing formation of intact assembled epitope peptide. Cells are then washed with 1 ml of PBSM, and incubated for a further 2 hours at room temperature to allow endocytosis to occur. Following this, treated cells are used to gauge uptake, processing, and HLA-A2 presentation of assembled peptides. This is performed using Jurkat cells transfected with a T cell receptor recognizing ELAGIGILTV (SEQ ID NO:126) in the context of HLA-A2, where the read-out for Jurkat activation is the secretion of IL-2, as described by Haggerty et al., Assay Drug Dev. Technol., 2012, 10, 187-201.

The process illustrated by this Example can encompass numerous other embodiments, involving variations on aptamers and targeting, and the types of structures assembled as the products of pairs of haplomers. Thus, aptamers may target cell surface structures directly, or any other components of a succeeding sandwich structure. Alternate haplomer-assembled products can included peptides binding to any other MHC class, or structures designed for direct recognition by antibodies. In the latter class of embodiments, such haplomer-assembled compounds include natural peptides, peptidomimetic structures, or non-peptide small organic molecules. Antibodies targeting such aptamer-mediated structures assembled from haplomers via the template assembly process can in turn promote target cell killing in various ways, including, but not limited to, antibody-dependent cellular cytotoxicity, complement pathways, or via antibody conjugates with highly cytotoxic drugs, including, but limited to, calicheamycin A and emtansine.

### Example 11: Hybridization-Mediated Positioning of a Probe Sequence on a Cell Surface Expressing a Specific Marker

This example demonstrates the efficacy of placement of a surface template on a cell surface in an in vitro system, as assessed by hybridization with a bilabeled fluorescent probe sequence.

An initial step in the placement of a surface template is the identification of a suitable marker that is expressed on the surface of the cells of interest. In this in vitro demonstration, Class I MHC was exploited, owing to its significant expression levels on the chosen target cells, a human EBV-transformed B lymphoid cell line. Following incubation of cells (10⁶/ml) with a biotin conjugate of primary antibody pan-specific for human Class I (W6/32) and washing in phosphate-buffered saline (pH 7.2; PBS), the cell preparation was treated with a large excess of unmodified tetravalent streptavidin (Sigma; 10 μl each of 100 μM streptavidin per 100 μl of cells at 10⁶/ml) for 30 minutes at room temperature, and rewashed in PBS. Since the streptavidin is applied in excess and is tetravalent, streptavidin which is bound to the cell surface (via the biotinylated primary antibody) still has available binding sites for biotin moieties on other molecules.

Following this, a biotinylated template oligonucleotide was added, such that its terminal biotin group becomes bound to free sites on the surface streptavidin. In principle, virtually any nucleic sequence can be used, with a wide range of modified phosphodiester backbones to confer nuclease resistance, including, but not limited to, phosphothioate, morpholinos, and 2′-O-methyl analogs. The DNA template sequence used in this example corresponds to a transcribed segment of human papillomavirus (HPV), of sequence: 5′-Biotin-TAACTGTCAAAAGCCACTGTGTC CTGAAGAAAAGCAAAGACATCTGGACAAAAAGC (SEQ ID NO:127). As a control, a scrambled version of this sequence was used: 5′ Biotin-TAGCGCAAATAAGCCGCCAGAAC GATGATATAAACAGCATTAGGTAAGCTACAACA (SEQ ID NO:128).

Following incubation of cells with biotinylated template or scrambled control, cells were washed with PBS, and subsequently treated with an oligonucleotide probe complementary to the surface HPV template and bearing fluorescent FAM labels at both 5′ and 3′ ends: 5′-FAM-TCCAGATGTCTTTGCTTTCTTCAGGACACA-3′-FAM (SEQ ID NO:119). In addition to this, another oligonucleotide with the same sequence may be used as a control to directly demonstrate the presence of surface streptavidin independently of hybridization, by virtue of its possession of a 5′-biotin and 3′-FAM moieties: 5′-Biotin-TCCAGATGTCTTTGC TTTCTTCAGGACACA-3′-FAM (SEQ ID NO:129).

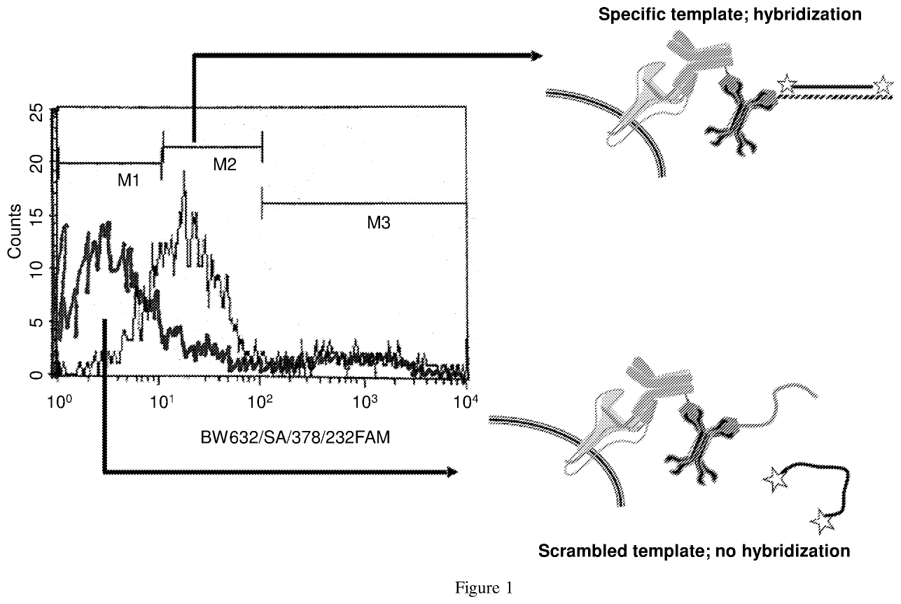

Following treatment with the probe oligonucleotide and washing with PBS, cell samples were analyzed by flow cytometry using standard settings for monitoring fluorescein-derived fluorescence, the results of which are shown in FIG. 1. A significant signal was observed with cells displaying the template complementary to the bilabeled probe sequence, but not with the control scrambled sequence (see, FIG. 1).

### Example 12: In Vitro Positioning of a Trastuzumab Mimotope on a HER-2 Negative Cell Via a Streptavidin Bridge

This example demonstrates the placement of a mimotope for the therapeutic antibody trastuzumab, which recognizes the protein HER-2, on the surface of a HER-2-negative cell, in an in vitro system.

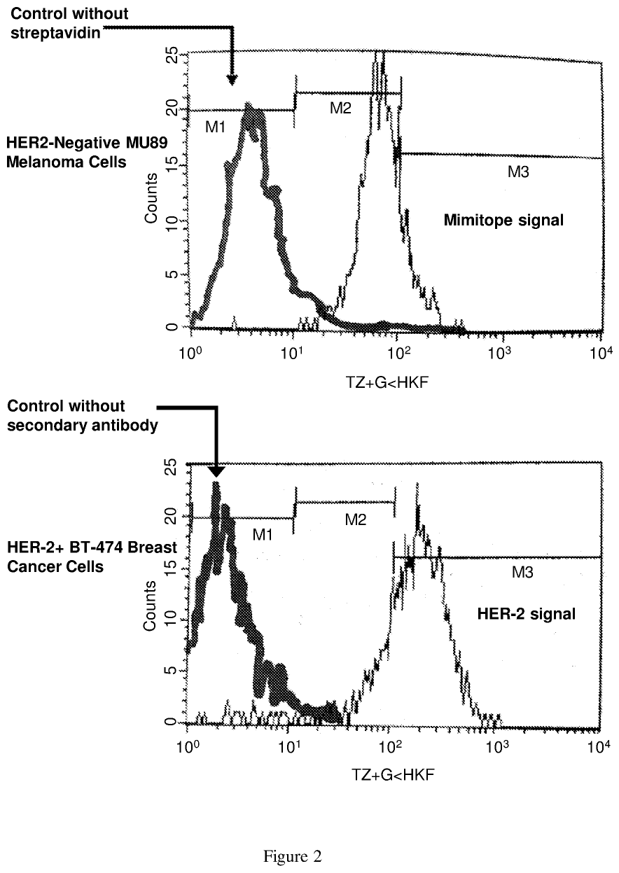

In common with Example 11, for the repurposing of an antibody epitope by placement of a peptide mimotope on a cell surface, a surface marker for the cell type of interest was defined. For this Example, the same biotinylated primary antibody (W6/32) as for Example 11 was used, but instead the melanoma cell line MU89 was chosen as the target, having been previously demonstrated to be negative for surface HER-2 expression. The complex (biotinylated template-streptavidin-primary antibody) was then assembled on MU89 surface. Following that procedure and washing, biotinylated mimotope (Biotin-SGGGSGGGQLGPY ELWELSH; SEQ ID NO:35) was allowed to bind to the surface streptavidin. The next step (post-washing) involved treatment with trastuzumab, for binding to the surface-linked mimotope. Subsequently, since trastuzumab is a humanized antibody with kappa light chains, a fluoresceinated goat anti-kappa antibody served to provide the final fluorescent read-out enabling flow analysis. Results showed that the surface-anchored mimotope elicited a clear-cut fluorescent signal from trastuzumab (see, FIG. 2). The positive control breast cancer cell line BT-474, known to express very high levels of HER-2 (see, FIG. 2), showed significantly stronger fluorescence than the mimotope signal from MU89. However, it should be noted that while an amplification effect for fluorescent signal results from the multi-layered sandwich technique of this approach, the overall signal is ultimately linked to the level of expression of the primary antibody target.

### Example 13: Preparation of Conjugates Between Split Epitope Segments and Nucleic Acid Strands Complementary to a Desired Template (Preparation of Split Epitope Haplomers)

This example demonstrates the formation of conjugates between specific split epitope segments and desired nucleic acid strands, that are complementary to a suitable template sequence.

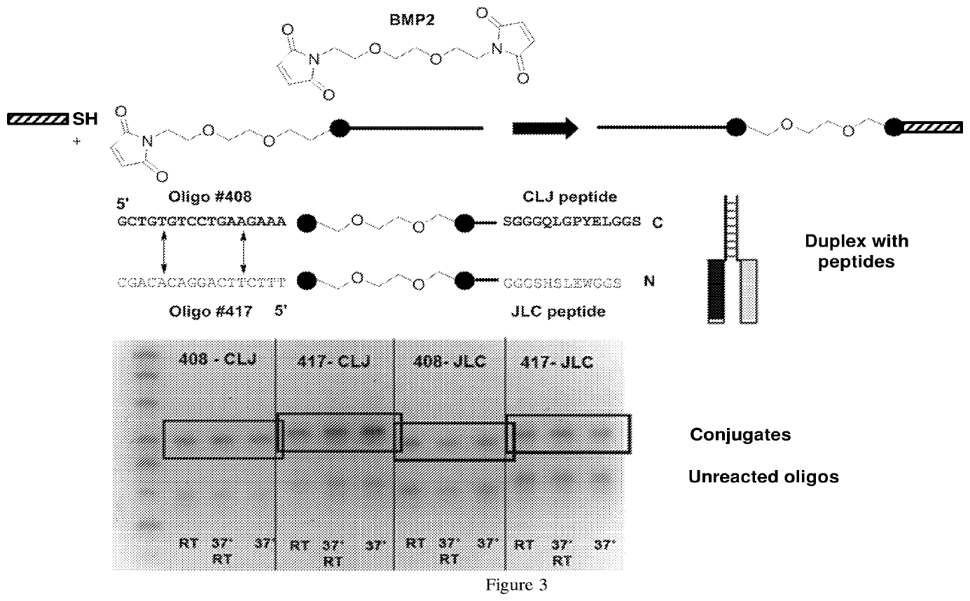

Conjugates were produced by means of a bis-maleimide (PEG)₂ compound (BMP2, Sigma; see FIG. 3), to form a covalent linkage between a 5′ or 3′ thiol on an oligonucleotide and a thiol from a reduced cysteine residue on a peptide. In the initial step, synthesized oligos with a disulfide protecting group were reduced with 10 mM Tris-carboxyethylphosphine (TCEP) for 16 hours, and then desalted by passage through P6 spin columns into 10 mM Tris pH 7.4 (BioRad). Following this, the reduced —SH oligonucleotides (typically 5 nmol) were reacted with a large molar excess (×120-fold) of BMP2, to drive monoderivatization of oligonucleotides rather than cross-linking. The reaction was performed in 50 mM phosphate buffer pH 7.0/100 mM NaCl for 4.5 hours at room temperature, followed by two tandem successive purifications through P6 microspin desalting columns (BioRad).

Peptides of interest bearing either N-terminal or C-terminal cysteine residues are then reacted with BMP2-modified oligonucleotides. The following mutually complementary oligonucleotides were used, both of which bear a 6-carbon spacer between terminal nucleotides and appended thiols (TriLink): #408: GCTGTGTCCTGAAGAAA-SH (SEQ ID NO:130) and #417: HS-TTTCTTCAGGACACAGC-[biotin] (SEQ ID NO:131). Oligonucleotide #417 also bears a 3′ biotin group for the application of subsequent binding assays, such as by ELISA (see Example 14).

The following peptides were used, as non-limiting examples of segments of the mimotope QLGPYELWELSH (SEQ ID NO:36), with intervening linker sequences: CL-JmimN-CSGGGQLGPYELGGS (SEQ ID NO:132) and JmimC-LC: SGGWELSHSGGGC (SEQ ID NO:133). The first peptide, CL-JmimN, bears an N-terminal cysteine, and the second (JmimC-LC) has a C-terminal cysteine.

For final conjugation reactions, 625 pmol of BMP2-modified oligonucleotides #408 and #417 were separately reacted with 2500 pmol of CL-JmimN or JmimC-LC (4-fold peptide excess) under varying conditions: room temperature and 37° C. for 16 hours, and 37° C. for 1 hour, followed by room temperature for 15 hours.

Following these incubations, 1.0 μl samples (12.5 pmol in terms of oligonucleotide amounts) were tested on a 15% denaturing 8 M urea gel (see, FIG. 3). Staining with SYBR-Gold visualizes both unreacted BMP2-derivatized oligonucleotides and oligonucleotide-peptide conjugates. Conjugate bands migrate more slowly than unconjugated material, affording the opportunity for their purification by virtue of their differing molecular weights (see, FIG. 3).

Purification can be achieved in various ways, including, but not limited to, non-denatuing gel electrophoresis, HPLC, and size-exclusion chromatography.

### Example 14: ELISA Assays for Mimotopes Using Trastuzumab, and Epitope Assembly Demonstration In Vitro

This example discloses a protocol for performing ELISAs for binding of mimotopes by antibodies of interest, and for demonstrating templated epitope assembly in vitro.

For intact mimotopes and their analogs, peptides with an N-terminal biotin label and a flexible spacer composed of serine and glycine residues (typically SGGGSGGG; SEQ ID NO:110) were used. A non-limiting example of an intact mimotope for trastuzumab for ELISA has the sequence: Biotin-SGGGSGGGQLGPYELWELSH (SEQ ID NO:35).

ELISA plates coated with tetravalent streptavidin were obtained for the assay, either from a commercial source (such as Thermofisher) or made by incubating polystyrene 96-well ELISA plates with solutions of streptavidin in PBS (1 μM) 16 hours/4° C., followed by blocking with a solution of bovine serum albumin (BSA) at 10 mg/ml in PBS pH 7.2, also for 16 hours/4° C. Plates were then emptied of solutions and washed three times with 100 μl PBS containing 0.05% Tween 20 (PBS-Tw20).

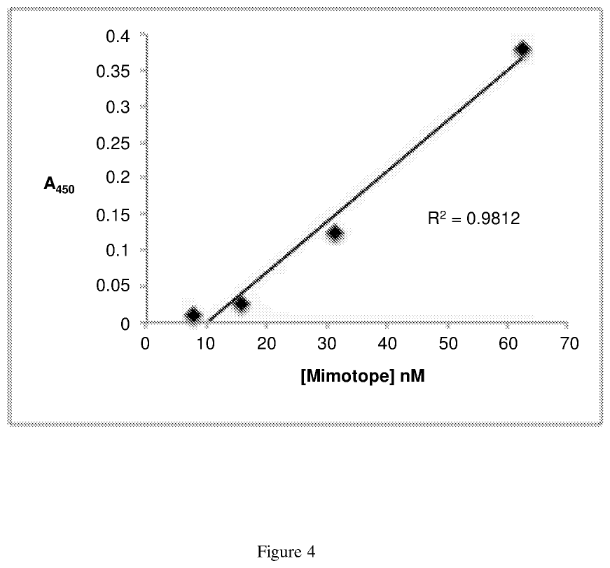

A suitable dilution series of the biotinylated peptides of interest was prepared in PBS, and at least duplicates (or higher numbers of replicates) were added to pre-designated wells in 100 μl volumes, and the plate incubated for 2 hours at room temperature (covered in Saran wrap). After this, the plate was emptied and washed ×5 with 100 μl PBS-Tw20, and the antibody being tested for binding to the mimotope was added at the appropriate dilution in PBS at 100 μl per well. In the present non-limiting example, the initial antibody was trastuzumab (BioVision), typically at a 1:500 dilution. Following another 1 hour at room temperature, the plate was emptied and washed ×2 with PBS-Tw20, and the appropriate dilution of the final antibody conjugate was added. In this specific example, the final antibody was a goat anti-human kappa light chain conjugate with horse radish peroxidase (HRP), since trastuzumab's light chain is of the kappa class. A typical working concentration of the anti-kappa-HRP conjugate was 1:5000, added to each well in 100 After another 1 hour room temperature incubation of the covered plate, it was then washed ×6 with 100 μl PBS-Tw20. At this point, 100 μl of TMB peroxide development reagent was added (as a 50:50 mix of two components of a commercial preparation (Becton-Dickinson, TMB Substrate Component Kit)). After a 30 minute incubation, reactions were stopped with 1 M sulfuric acid, and results assessed with an automated plate reader for absorbances at 450 nm. Final data was compiled from averaged replicates for each determination with subtraction of absorbances observed in wells where no target peptide antigen was added. Standard curves for unmodified mimotope signal from trastuzumab were thus constructed (see, FIG. 4).

For ELISA testing of antibody recognition of split epitopes, a non-limiting strategy involves using hybridizations involving peptide epitope segments conjugated to nucleic acid oligonucleotides (Example 3), as an implementation of TAPER technology. In one such embodiment, the oligonucleotides are mutually complementary. Alternative architectures are available, including, but not limited to, where both oligonucleotides hybridize to a common template immobilized by means of streptavidin-biotin binding, as above.

### Example 15: Epitope Assembly on Cell Surfaces by Means of Surface Template and Targeted Epitope Haplomers (Prophetic)

This example discloses a procedure for the cell surface templating of epitope haplomers, for the generation and subsequent recognition of reconstituted epitope sequences. Specific surface targets on pathological cells of interest are chosen from available knowledge in the state of the art, or obtained from experimental results, as exemplified by an aptamer-based subtraction process. Where targets afford a ligand-based interaction system, a ligand-tag strategy may be used; alternatively the target may be used for the development of an aptamer-based strategy where aptamers binding to specific surface targets function in a dual capacity as both recognition elements and template-display systems.

Once a system for display of surface template is developed, then the template can act as specific site for hybridization, as demonstrated experimentally. If the template and hybridizing haptamer nucleic acid moieties are composed of DNA of opposite chirality (L-DNA) to normal, then bio-orthogonal hybridization can be achieved (Aptamer patent). Epitope assembly by means of proximal hybridizations of haplomers bearing epitope segments is then effected in an analogous manner to similar processes conducted in vitro.

### Example 16: Epitope Binding by Trastuzumab

Bioinylated unmodified mimotope (Bio-SGGGSGGGQLGPYELWELSH; SEQ ID NO:35) and a corresponding cysteine-modified mimotope (Bio-SGGGSGGGQLGPYELWEL CH; SEQ ID NO:3) were assayed in parallel by ELISA using the basic protocol as for Example 14.

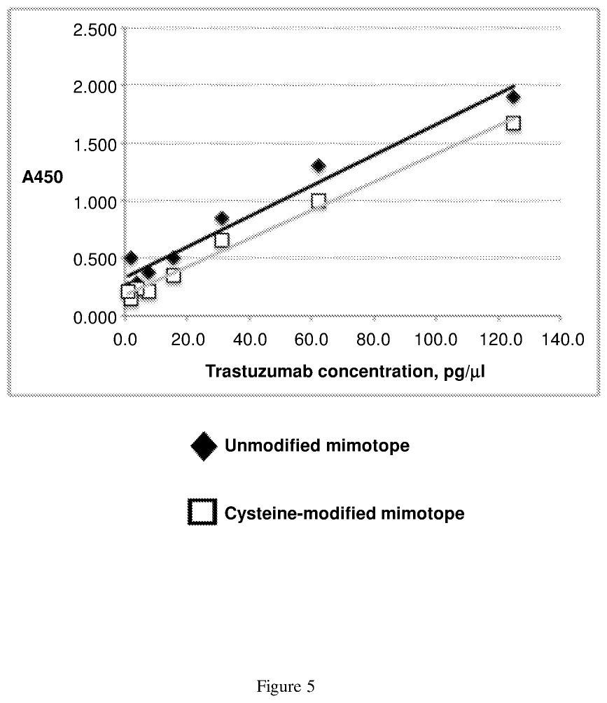

The wells in the streptavidin-coated plates were coated with 100 μl of 400 nM biotinylated peptides in PBS, or PBS only as controls, for 2 hours at room temperature. Trastuzumab was then titrated for both peptides, in duplicate 2-fold serial dilutions (100 μl per well) for 1 hour at room temperature. PBS controls (without peptide) were treated with the lowest dilutions of Trastuzumab. Signal detection with HRP-conjugated goat anti-human kappa chain was then as for Example 14, except the latter HRP-antibody was used at 1 1:7500 dilution. Data was corrected for background levels seen in wells with Trastuzumab only, and plotted as shown in FIG. 5.

The results show a similar titration curve for both peptides, where the slope of the cysteine peptide is approximately 10% less than that seen for the unmodified peptide (confirmed in a repeated experiment), indicated that Trastuzumab recognizes the cysteine-modified mimotope to almost the same extent as the unmodified mimotope.

Various modifications of the described subject matter, in addition to those described herein, will be apparent to those skilled in the art from the foregoing description. Such modifications are also intended to fall within the scope of the appended claims Each reference (including, but not limited to, journal articles, U.S. and non-U.S. patents, patent application publications, international patent application publications, gene bank accession numbers, and the like) cited in the present application is incorporated herein by reference in its entirety.

1. An isolated polypeptide comprising the formula: SerGlyGlyGlySerGlyGlyGly GlnLeuXaa¹ProTyrGluXaa²TrpGluLeuXaa³His, wherein one of:
Xaa¹ is Cys; Xaa² is Leu; and Xaa³ is Ser;
Xaa¹ is Gly; Xaa² is Cys; and Xaa³ is Ser; or
Xaa¹ is Gly; Xaa² is Leu; and Xaa³ is Cys.

2. (canceled)

3. An isolated polypeptide comprising the formula: SerGlyGlyGlySerGlyGlyGly GlnXaa¹LeuXaa²GlyXaa³ProXaa⁴TyrXaa⁵GluXaa⁶LeuXaa⁷TrpXaa⁸GluXaeLeuXaa¹⁰SerXaa¹¹His, wherein one of:
Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent;
Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent;
Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent;
Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent;
Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent;
Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent;
Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are ab sent;
Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are ab sent;
Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are ab sent;
Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent; or
Xaa¹¹ is Cys and Xaa¹, Xaa², Xaa³, Xaa⁴, Xaa⁵, Xaa⁶, Xaa⁷, Xaa⁸, Xaa⁹, and Xaa¹¹ are absent.

4. (canceled)

5. A composition comprising a pair of polypeptides, wherein the pair of polypeptides is:
a) SerGlyGlyGlySerGlyGlyGlyGlnLeu and Xaa¹ProTyrGluXaa²TrpGluLeuXaa³His, wherein Xaa¹ is Cys, Xaa² is Leu, and Xaa³ is Ser;
b) SerGlyGlyGlySerGlyGlyGlyGlnLeuXaa¹ProTyrGlu and Xaa²TrpGluLeuXaa³His, wherein Xaa¹ is Gly, Xaa² is Cys, and Xaa³ is Ser; or
c) SerGlyGlyGlySerGlyGlyGlyGlnLeuXaa¹ProTyrGluXaa²TrpGluLeu and Xaa³His, wherein Xaa¹ is Gly, Xaa² is Leu, and Xaa³ is Cys.

6. The composition of claim 5, wherein the C-terminus of the first polypeptide further comprises a first bio-orthogonal reactive group and the N-terminus of the second polypeptide further comprises a second bio-orthogonal reactive group, wherein the first bio-orthogonal reactive group and the second bio-orthogonal reactive group are compatible.

7. The composition of claim 6, wherein:
the first bio-orthogonal reactive group is a linear alkyne and the second bio-orthogonal reactive group is an azide, or the second bio-orthogonal reactive group is a linear alkyne and the first bio-orthogonal reactive group is an azide;
the first bio-orthogonal reactive group is a strained alkyne and the second bio-orthogonal reactive group is an azide or the second bio-orthogonal reactive group is a strained alkyne and the first bio-orthogonal reactive group is an azide; or
the first bio-orthogonal reactive group is a tetrazine and the second bio-orthogonal reactive group is a cyclooctene or the second bio-orthogonal reactive group is a tetrazine and the first bio-orthogonal reactive group is a cyclooctene.

8. The composition of claim 5, wherein the C-terminus of the first polypeptide further comprises a first chemical modification and the N-terminus of the second polypeptide further comprises a second chemical modification, wherein the chemical modification and the second chemical modification are compatible.

9. The composition of claim 8, wherein:
the first chemical modification is amidation (CONH₂) or esterification (COOR), where R is methyl, ethyl, or phenyl; and
the second chemical modification is acetylation or an N-methyl substitution of the N-terminal amino group.

10. A composition comprising a pair of polypeptides, wherein the pair of polypeptides is:
a) SerGlyGlyGlySerGlyGlyGlyGln and Xaa¹LeuXaa²GlyXaa³ProXaa⁴TyrXaa⁵GluXaa⁶LeuXaa⁷TrpXaa⁸GluXaa⁹LeuXaa¹⁰SerXaa¹¹His, wherein Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent;
b) SerGlyGlyGlySerGlyGlyGlyGlnXaa¹Leu and Xaa²GlyXaa³ProXaa⁴TyrXaa⁵GluXaa⁶LeuXaa⁷TrpXaa⁸GluXaa⁹LeuXaa¹⁰SerXaa¹¹His, wherein Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent;
c) SerGlyGlyGlySerGlyGlyGlyGlnXaa¹LeuXaa²Gly and Xaa³ProXaa⁴TyrXaa⁵GluXaa⁶LeuXaa⁷TrpXaa⁸GluXaa⁹LeuXaa¹⁰SerXaa¹¹His, wherein Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent;
d) SerGlyGlyGlySerGlyGlyGlyGlnXaa¹LeuXaa²GlyXaa³Pro and Xaa⁴TyrXaa⁵GluXaa⁶LeuXaa⁷TrpXaa⁸GluXaa⁹LeuXaa¹⁰SerXaa¹¹His, wherein Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent;
e) SerGlyGlyGlySerGlyGlyGlyGlnXaa¹LeuXaa²GlyXaa³ProXaa⁴Tyr and Xaa⁵GluXaa⁶LeuXaa⁷TrpXaa⁸GluXaa⁹LeuXaa¹⁰SerXaa¹¹His, wherein Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent;
f) SerGlyGlyGlySerGlyGlyGlyGlnXaa¹LeuXaa²GlyXaa³ProXaa⁴TyrXaa⁵Glu and Xaa⁶LeuXaa⁷TrpXaa⁸GluXaa⁹LeuXaa¹⁰SerXaa¹¹His, wherein Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent;
g) SerGlyGlyGlySerGlyGlyGlyGlnXaa¹LeuXaa²GlyXaa³ProXaa⁴TyrXaa⁵GluXaa⁶Leu and Xaa⁷TrpXaa⁸GluXaa⁹LeuXaa¹⁰SerXaa¹¹His, wherein Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent;
h) SerGlyGlyGlySerGlyGlyGlyGlnXaa¹LeuXaa²GlyXaa³ProXaa⁴TyrXaa⁵GluXaa⁶Leu Xaa⁷Trp and Xaa⁸GluXaa⁹LeuXaa¹⁰SerXaa¹¹His, wherein Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent;
i) SerGlyGlyGlySerGlyGlyGlyGlnXaa¹LeuXaa²GlyXaa³ProXaa⁴TyrXaa⁵GluXaa⁶Leu Xaa⁷TrpXaa⁸Glu and Xaa⁹LeuXaa¹⁰SerXaa¹¹His, wherein Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are absent;
j) SerGlyGlyGlySerGlyGlyGlyGlnXaa¹LeuXaa²GlyXaa³ProXaa⁴TyrXaa⁵GluXaa⁶Leu Xaa⁷TrpXaa⁸GluXaa⁹Leu and Xaa¹⁰SerXaa¹¹His, wherein Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent; or
k) SerGlyGlyGlySerGlyGlyGlyGlnXaa¹LeuXaa²GlyXaa³ProXaa⁴TyrXaa⁵GluXaa⁶Leu Xaa⁷TrpXaa⁸GluXaa⁹LeuXaa¹⁰Ser and Xaa¹¹His, wherein Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent.

11. The composition of claim 10, wherein the C-terminus of the first polypeptide further comprises a first bio-orthogonal reactive group and the N-terminus of the second polypeptide further comprises a second bio-orthogonal reactive group, wherein the first bio-orthogonal reactive group and the second bio-orthogonal reactive group are compatible.

12. The composition of claim 11, wherein:
the first bio-orthogonal reactive group is a linear alkyne and the second bio-orthogonal reactive group is an azide, or the second bio-orthogonal reactive group is a linear alkyne and the first bio-orthogonal reactive group is an azide;
the first bio-orthogonal reactive group is a strained alkyne and the second bio-orthogonal reactive group is an azide or the second bio-orthogonal reactive group is a strained alkyne and the first bio-orthogonal reactive group is an azide; or
the first bio-orthogonal reactive group is a tetrazine and the second bio-orthogonal reactive group is a cyclooctene or the second bio-orthogonal reactive group is a tetrazine and the first bio-orthogonal reactive group is a cyclooctene.

13. The composition of claim 10, wherein the C-terminus of the first polypeptide further comprises a first chemical modification and the N-terminus of the second polypeptide further comprises a second chemical modification, wherein the chemical modification and the second chemical modification are compatible.

14. The composition of claim 13, wherein:
the first chemical modification is amidation (CONH₂) or esterification (COOR), where R is methyl, ethyl, or phenyl; and
the second chemical modification is acetylation or an N-methyl substitution of the N-terminal amino group.

15. (canceled)

16. A method for the directed assembly of an epitope on a target cell for a recognition molecule comprising:
a) contacting the target cell with a singlet aptamer, wherein the singlet aptamer comprises:
i) a first portion folded into a tertiary structure that is able to bind to a target molecule on the surface of the target cell; and
ii) a second portion comprising a nucleic acid molecule linked to the first portion at either the 3′ or 5′ terminal end of the second portion; and
b) contacting the target cell with a first epitope haplomer and a second epitope haplomer;
wherein the first epitope haplomer comprises:
i) a nucleic acid molecule that is complementary to the second portion of the singlet aptamer; and
ii) a reactive effector moiety that is a first portion of the epitope;
wherein the second epitope haplomer comprises:
i) a nucleic acid molecule that is complementary to the second portion of the singlet aptamer; and
ii) a reactive effector moiety that is a second portion of the epitope;
wherein the nucleic acid molecule of the first epitope haplomer is complementary to a region of the second portion of the singlet aptamer that is in spatial proximity to the region of the second portion of the singlet aptamer to which the nucleic acid molecule of the second epitope haplomer is complementary; and
wherein the reactive effector moiety of the first epitope haplomer is in spatial proximity to the reactive effector moiety of the second epitope haplomer, thereby resulting in the directed assembly of the epitope.

17. A method for the directed assembly of an epitope on a target cell for a recognition molecule comprising:
a) contacting the target cell with a dual proximal aptamer pair, wherein the dual proximal aptamer pair comprises a first aptamer and a second aptamer, wherein:
the first aptamer comprises:
i) a first portion folded into a tertiary structure that is able to bind to a target molecule on the surface of the target cell; and
ii) a second portion comprising a nucleic acid molecule linked to the first portion at either the 3′ or 5′ terminal end of the second portion; and
the second aptamer comprises:
i) a first portion folded into a tertiary structure that is able to bind to a target molecule on the surface of the target cell; and
ii) a second portion comprising a nucleic acid molecule linked to the first portion at either the 3′ or 5′ terminal end of the second portion; and
b) contacting the target cell with a first epitope haplomer and a second epitope haplomer;
wherein the first epitope haplomer comprises:
i) a nucleic acid molecule that is complementary to the second portion of the first aptamer; and
ii) a reactive effector moiety that is a first portion of the epitope;
wherein the second epitope haplomer comprises:
i) a nucleic acid molecule that is complementary to the second portion of the second aptamer; and
ii) a reactive effector moiety that is a second portion of the epitope;
wherein the nucleic acid molecule of the first epitope haplomer is complementary to a region of the second portion of the first aptamer that is in spatial proximity to the region of the second portion of the second aptamer to which the nucleic acid molecule of the second epitope haplomer is complementary; and
wherein the reactive effector moiety of the first epitope haplomer is in spatial proximity to the reactive effector moiety of the second epitope haplomer, thereby resulting in the directed assembly of the epitope.

18. The method of claim 17, wherein both aptamers bind to the same target molecule such that the aptamer pair is in physical proximity.

19. The method of claim 17, wherein each aptamer binds to a different target molecule on the same cell such that the aptamer pair is in physical proximity.

20. (canceled)

21. A method for the directed assembly of an epitope on a target cell for a recognition molecule comprising:
a) contacting the target cell with a binary aptamer, wherein the binary aptamer comprises:
i) a first portion folded into a tertiary structure that is able to bind to a target molecule on the surface of the target cell;
ii) a second portion folded into a tertiary structure that is able to bind to a target molecule on the surface of the target cell; and
iii) a third portion comprising a nucleic acid molecule located between the first and second portion; and
b) contacting the target cell with a first epitope haplomer and a second epitope haplomer;
wherein the first epitope haplomer comprises:
i) a nucleic acid molecule that is complementary to the third portion of the binary aptamer; and
ii) a reactive effector moiety that is a first portion of the epitope;
wherein the second epitope haplomer comprises:
i) a nucleic acid molecule that is complementary to the third portion of the binary aptamer; and
ii) a reactive effector moiety that is a second portion of the epitope;
wherein the nucleic acid molecule of the first epitope haplomer is complementary to a region of the third portion of the binary aptamer that is in spatial proximity to the region of the third portion of the binary aptamer to which the nucleic acid molecule of the second epitope haplomer is complementary; and
wherein the reactive effector moiety of the first epitope haplomer is in spatial proximity, to the reactive effector moiety of the second epitope haplomer, thereby resulting in the directed assembly of the epitope.

22. The method of claim 21, wherein the first portion of the binary aptamer and the second portion of the binary aptamer are both nucleic acid molecules, wherein each nucleic acid molecule comprises about 20 nucleotides to about 80 nucleotides in length and have a Tm from about 55° to about 65° C., and the third portion of the binary aptamer located between the first portion and second portion comprises from about 40 nucleotides to about 60 nucleotides in length.

23. The method of claim 16, wherein any one or more of the nucleic acid molecules comprises DNA nucleotides, RNA nucleotides, phosphorothioate-modified nucleotides, 2-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, or other nucleic acid analogues capable of base-pair formation, or any combination thereof.

24-32. (canceled)

33. The method of claim 16, wherein:
a) one of the reactive effector moiety of the first epitope haplomer and the reactive effector moiety of the second epitope haplomer is SerGlyGlyGlySerGlyGlyGlyGlnLeu and the other of the reactive effector moiety of the first epitope haplomer and the reactive effector moiety of the second epitope haplomer is Xaa¹ProTyrGluXaa²TrpGluLeuXaa³His, wherein Xaa¹ is Cys, Xaa² is Leu, and Xaa³ is Ser;
b) one of the reactive effector moiety of the first epitope haplomer and the reactive effector moiety of the second epitope haplomer is SerGlyGlyGlySerGlyGlyGlyGlnLeuXaa¹ProTyrGlu and the other of the reactive effector moiety of the first epitope haplomer and the reactive effector moiety of the second epitope haplomer is Xaa²TrpGluLeuXaa³His, wherein Xaa¹ is Gly, Xaa² is Cys, and Xaa³ is Ser;
c) one of the reactive effector moiety of the first epitope haplomer and the reactive effector moiety of the second epitope haplomer is SerGlyGlyGlySerGlyGlyGlyGlnLeuXaa¹ProTyrGluXaa²TrpGluLeu and the other of the reactive effector moiety of the first epitope haplomer and the reactive effector moiety of the second epitope haplomer is Xaa³His, wherein Xaa¹ is Gly, Xaa² is Leu, and Xaa³ is Cys;
d) one of the reactive effector moiety of the first epitope haplomer and the reactive effector moiety of the second epitope haplomer is SerGlyGlyGlySerGlyGlyGlyGln and the other of the reactive effector moiety of the first epitope haplomer and the reactive effector moiety of the second epitope haplomer is Xaa¹LeuXaa²GlyXaa³ProXaa⁴TyrXaa⁵GluXaa⁶Leu Xaa⁷TrpXaa⁸GluXaeLeuXaa¹⁰SerXaa¹¹His, wherein Xaa¹ is Cys and Xaa², Xaa³, Xaa⁴, Xaa⁵, Xaa⁶, Xaa⁷, Xaa⁸, Xaa⁹, Xaa¹⁰, and Xaa¹¹ are absent;
e) one of the reactive effector moiety of the first epitope haplomer and the reactive effector moiety of the second epitope haplomer is SerGlyGlyGlySerGlyGlyGlyGlnXaa¹Leu and the other of the reactive effector moiety of the first epitope haplomer and the reactive effector moiety of the second epitope haplomer is Xaa²GlyXaa³ProXaa⁴TyrXaa⁵GluXaa⁶Leu Xaa⁷TrpXaa⁸GluXaa⁹LeuXaa¹⁰SerXaa¹¹His, wherein Xaa² is Cys and Xaa¹, Xaa³, Xaa⁴, Xaa⁵, Xaa⁶, Xaa⁷, Xaa⁸, Xaa⁹, Xaa¹⁰, and Xaa¹¹ are absent;
f) one of the reactive effector moiety of the first epitope haplomer and the reactive effector moiety of the second epitope haplomer is SerGlyGlyGlySerGlyGlyGlyGlnXaa¹Leu Xaa²Gly and the other of the reactive effector moiety of the first epitope haplomer and the reactive effector moiety of the second epitope haplomer is Xaa³ProXaa⁴TyrXaa⁵GluXaa⁶Leu Xaa⁷TrpXaa⁸GluXaa⁹LeuXaa¹⁰SerXaa¹¹His, wherein Xaa³ is Cys and Xaa¹, Xaa², Xaa⁴, Xaa⁵, Xaa⁶, Xaa⁷, Xaa⁸, Xaa⁹, Xaa¹⁰, and Xaa¹¹ are absent;
g) one of the reactive effector moiety of the first epitope haplomer and the reactive effector moiety of the second epitope haplomer is SerGlyGlyGlySerGlyGlyGlyGlnXaa¹Leu Xaa²GlyXaa³Pro and the other of the reactive effector moiety of the first epitope haplomer and the reactive effector moiety of the second epitope haplomer is Xaa⁴TyrXaa⁵GluXaa⁶Leu Xaa⁷TrpXaa⁸GluXaa⁹LeuXaa¹⁰SerXaa¹¹His, wherein Xaa⁴ is Cys and Xaa¹, Xaa², Xaa³, Xaa⁵, Xaa⁶, Xaa⁷, Xaa⁸, Xaa⁹, Xaa¹⁰, and Xaa¹¹ are absent;
h) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴Tyr and the other of the reactive effector moiety of the first epitope haplomer and the reactive effector moiety of the second epitope haplomer is Xaa⁵GluXaa⁶LeuXaa⁷TrpXaa⁸GluXaa⁹LeuXaa¹⁰SerXaa¹¹His, wherein Xaa⁵ is Cys and Xaa¹, Xaa², Xaa³, Xaa⁴, Xaa⁶, Xaa⁷, Xaa⁸, Xaa⁹, Xaa¹⁰, and Xaa¹¹ are absent;
i) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵Glu and the other of the reactive effector moiety of the first epitope haplomer and the reactive effector moiety of the second epitope haplomer is Xaa⁶Leu Xaa⁷TrpXaa⁸GluXaa⁹LeuXaa¹⁰SerXaa¹¹His, wherein Xaa⁶ is Cys and Xaa¹, Xaa², Xaa³, Xaa⁴, Xaa⁵, Xaa⁷, Xaa⁸, Xaa⁹, Xaa¹⁰, and Xaa¹¹ are absent;
j) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶Leu and the other of the reactive effector moiety of the first epitope haplomer and the reactive effector moiety of the second epitope haplomer is Xaa⁷TrpXaa⁸GluXaa⁹LeuXaa¹⁰SerXaa¹¹His, wherein Xaa⁷ is Cys and Xaa¹, Xaa², Xaa³, Xaa⁴, Xaa⁵, Xaa⁶, Xaa⁸, Xaa⁹, Xaa¹⁰, and Xaa¹¹ are absent;
k) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶LeuXaa⁷Trp and the other of the reactive effector moiety of the first epitope haplomer and the reactive effector moiety of the second epitope haplomer is Xaa⁸GluXaa⁹LeuXaa¹⁰SerXaa¹¹His, wherein Xaa⁸ is Cys and Xaa¹, Xaa², Xaa³, Xaa⁴, Xaa⁵, Xaa⁶, Xaa⁷, Xaa⁹, Xaa¹⁰, and Xaa¹¹ are absent;
l) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶LeuXaa⁷TrpXaa⁸Glu and the other of the reactive effector moiety of the first epitope haplomer and the reactive effector moiety of the second epitope haplomer is Xaa⁹LeuXaa¹⁰SerXaa¹¹His, wherein Xaa⁹ is Cys and Xaa¹, Xaa², Xaa³, Xaa⁴, Xaa⁵, Xaa⁶, Xaa⁷, Xaa⁸, Xaa¹⁰, and Xaa¹¹ are absent;
m) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶LeuXaa⁷TrpXaa⁸GluXaa⁹Leu and the other of the reactive effector moiety of the first epitope haplomer and the reactive effector moiety of the second epitope haplomer is Xaa¹⁰SerXaa¹¹His, wherein Xaa¹⁰ is Cys and Xaa¹, Xaa², Xaa³, Xaa⁴, Xaa⁵, Xaa⁶, Xaa⁷, Xaa⁸, Xaa⁹, and Xaa¹¹ are absent; or
n) one of the reactive effector moiety of the first epitope haplomer and the reactive effector moiety of the second epitope haplomer is SerGlyGlyGlySerGlyGlyGlyGlnXaa¹Leu Xaa²GlyXaa³ProXaa⁴TyrXaa⁵GluXaa⁶LeuXaa⁷TrpXaa⁸GluXaa⁹LeuXaa¹⁰Ser and the other of the reactive effector moiety of the first epitope haplomer and the reactive effector moiety of the second epitope haplomer is Xaa¹¹His, wherein Xaa¹¹ is Cys and Xaa¹, Xaa², Xaa³, Xaa⁴, Xaa⁵, Xaa⁶, Xaa⁷, Xaa⁸, Xaa⁹, and Xaa¹⁰ are absent.

34-36. (canceled)


SEQUENCE LISTING (133 sequences)

SEQ ID NO: 1 - protein, 20 aa, artificial sequence
  polypeptide
SGGGSGGGQLCPYELWELSH

SEQ ID NO: 2 - protein, 20 aa, artificial sequence
  polypeptide
SGGGSGGGQLGPYECWELSH

SEQ ID NO: 3 - protein, 20 aa, artificial sequence
  polypeptide
SGGGSGGGQLGPYELWELCH

SEQ ID NO: 4 - protein, 21 aa, artificial sequence
  polypeptide
SGGGSGGGQCLGPYELWELSH

SEQ ID NO: 5 - protein, 21 aa, artificial sequence
  polypeptide
SGGGSGGGQLCGPYELWELSH

SEQ ID NO: 6 - protein, 21 aa, artificial sequence
  polypeptide
SGGGSGGGQLGCPYELWELSH

SEQ ID NO: 7 - protein, 21 aa, artificial sequence
  polypeptide
SGGGSGGGQLGPCYELWELSH

SEQ ID NO: 8 - protein, 21 aa, artificial sequence
  polypeptide
SGGGSGGGQLGPYCELWELSH

SEQ ID NO: 9 - protein, 21 aa, artificial sequence
  polypeptide
SGGGSGGGQLGPYECLWELSH

SEQ ID NO: 10 - protein, 21 aa, artificial sequence
  polypeptide
SGGGSGGGQLGPYELCWELSH

SEQ ID NO: 11 - protein, 21 aa, artificial sequence
  polypeptide
SGGGSGGGQLGPYELWCELSH

SEQ ID NO: 12 - protein, 21 aa, artificial sequence
  polypeptide
SGGGSGGGQLGPYELWECLSH

SEQ ID NO: 13 - protein, 21 aa, artificial sequence
  polypeptide
SGGGSGGGQLGPYELWELCSH

SEQ ID NO: 14 - protein, 21 aa, artificial sequence
  polypeptide
SGGGSGGGQLGPYELWELSCH

SEQ ID NO: 15 - protein, 10 aa, artificial sequence
  polypeptide
SGGGSGGGQL

SEQ ID NO: 16 - protein, 10 aa, artificial sequence
  polypeptide
CPYELWELSH

SEQ ID NO: 17 - protein, 14 aa, artificial sequence
  polypeptide
SGGGSGGGQLGPYE

SEQ ID NO: 18 - protein, 6 aa, artificial sequence
  polypeptide
CWELSH

SEQ ID NO: 19 - protein, 18 aa, artificial sequence
  polypeptide
SGGGSGGGQLGPYELWEL

SEQ ID NO: 20 - protein, 9 aa, artificial sequence
  polypeptide
SGGGSGGGQ

SEQ ID NO: 21 - protein, 12 aa, artificial sequence
  polypeptide
CLGPYELWELSH

SEQ ID NO: 22 - protein, 11 aa, artificial sequence
  epitope haplomer
CGPYELWELSH

SEQ ID NO: 23 - protein, 11 aa, artificial sequence
  epitope haplomer
SGGGSGGGQLG

SEQ ID NO: 24 - protein, 12 aa, artificial sequence
  epitope haplomer
SGGGSGGGQLGP

SEQ ID NO: 25 - protein, 9 aa, artificial sequence
  epitope haplomer
CYELWELSH

SEQ ID NO: 26 - protein, 13 aa, artificial sequence
  epitope haplomer
SGGGSGGGQLGPY

SEQ ID NO: 27 - protein, 8 aa, artificial sequence
  epitope haplomer
CELWELSH

SEQ ID NO: 28 - protein, 7 aa, artificial sequence
  epitope haplomer
CLWELSH

SEQ ID NO: 29 - protein, 15 aa, artificial sequence
  epitope haplomer
SGGGSGGGQLGPYEL

SEQ ID NO: 30 - protein, 16 aa, artificial sequence
  reactive effector moiety
SGGGSGGGQLGPYELW

SEQ ID NO: 31 - protein, 5 aa, artificial sequence
  reactive effector moiety
CELSH

SEQ ID NO: 32 - protein, 17 aa, artificial sequence
  reactive effector moiety
SGGGSGGGQLGPYELWE

SEQ ID NO: 33 - protein, 4 aa, artificial sequence
  reactive effector moiety
CLSH

SEQ ID NO: 34 - protein, 19 aa, artificial sequence
  reactive effector moiety
SGGGSGGGQLGPYELWELS

SEQ ID NO: 35 - protein, 20 aa, artificial sequence
  mimotope
SGGGSGGGQLGPYELWELSH

SEQ ID NO: 36 - protein, 12 aa, artificial sequence
  segment of the mimotope
QLGPYELWELSH

SEQ ID NO: 37 - protein, 12 aa, artificial sequence
  derivative of the initial mimotope
LLGPYELWELSH

SEQ ID NO: 38 - protein, 24 aa, artificial sequence
  native sequence of the epitope
NSELLSLINDMPITNDQKKLMSNN

SEQ ID NO: 39 - protein, 18 aa, artificial sequence
  N terminal epitope fragment
NSELLSLINDMGPSGGGS

SEQ ID NO: 40 - protein, 20 aa, artificial sequence
  alternate sequence
LTNSELLSLINDMGPSGGGS

SEQ ID NO: 41 - protein, 10 aa, artificial sequence
  alternate sequence at the C terminus
NSELLSLIND

SEQ ID NO: 42 - protein, 9 aa, artificial sequence
  epitope fragment sequence
MPITSGGGS

SEQ ID NO: 43 - protein, 9 aa, artificial sequence
  epitope fragment sequence
MGGSSGGGS

SEQ ID NO: 44 - protein, 9 aa, artificial sequence
  epitope fragment sequence
MGAPSGGGS

SEQ ID NO: 45 - protein, 9 aa, artificial sequence
  epitope fragment sequence
GGPSSGGGS

SEQ ID NO: 46 - protein, 9 aa, artificial sequence
  epitope fragment sequence
GPSGSGGGS

SEQ ID NO: 47 - protein, 17 aa, artificial sequence
  C terminal epitope fragment
SGGGGLSNDQKKLMSNN

SEQ ID NO: 48 - protein, 19 aa, artificial sequence
  alternate sequence
SGGGGLSNDQKKLMSNNVQ

SEQ ID NO: 49 - protein, 10 aa, artificial sequence
  alternate sequence
NDQKKLMSNN

SEQ ID NO: 50 - protein, 7 aa, artificial sequence
  epitope fragment sequence
SGGGGLS

SEQ ID NO: 51 - protein, 7 aa, artificial sequence
  epitope fragment sequence
SGGGGAS

SEQ ID NO: 52 - protein, 7 aa, artificial sequence
  epitope fragment sequence
SGGGGAP

SEQ ID NO: 53 - protein, 7 aa, artificial sequence
  epitope fragment sequence
SGGGGLD

SEQ ID NO: 54 - protein, 7 aa, artificial sequence
  epitope fragment sequence
SGGGGLN

SEQ ID NO: 55 - protein, 12 aa, artificial sequence
  EGF peptide sequence
IYPPLLRTSQAM

SEQ ID NO: 56 - protein, 12 aa, artificial sequence
  EGF peptide sequence
AYPPYLRSMTLY

SEQ ID NO: 57 - protein, 12 aa, artificial sequence
  EGF peptide sequence
YPPAERTYSTNY

SEQ ID NO: 58 - protein, 9 aa, artificial sequence
  EGF peptide sequence
CPKWDAARC

SEQ ID NO: 59 - protein, 9 aa, artificial sequence
  EGF peptide sequence
CGPTRWRSC

SEQ ID NO: 60 - protein, 12 aa, artificial sequence
  Vi antigen peptide
TSHHDSHGLHRV

SEQ ID NO: 61 - protein, 12 aa, artificial sequence
  Vi antigen peptide
TSHHDSHGDHHV

SEQ ID NO: 62 - protein, 12 aa, artificial sequence
  Vi antigen peptide
TSHHDSHGVHRV

SEQ ID NO: 63 - protein, 12 aa, artificial sequence
  Vi antigen peptide
TSHHDSHDLHRV

SEQ ID NO: 64 - protein, 12 aa, artificial sequence
  Vi antigen peptide
TSHHDYHGLHRV

SEQ ID NO: 65 - protein, 12 aa, artificial sequence
  Vi antigen peptide
ENHSPVNIAHKL

SEQ ID NO: 66 - protein, 12 aa, artificial sequence
  Vi antigen peptide
ENHSPVNIAHKV

SEQ ID NO: 67 - protein, 12 aa, artificial sequence
  Vi antigen peptide
ENHSPVNIDHKL

SEQ ID NO: 68 - protein, 12 aa, artificial sequence
  Vi antigen peptide
EDHSPVNIDHKL

SEQ ID NO: 69 - protein, 12 aa, artificial sequence
  Vi antigen peptide
ENHYPLHAAHRI

SEQ ID NO: 70 - protein, 12 aa, artificial sequence
  Vi antigen peptide
ESHQHVHDLVFL

SEQ ID NO: 71 - protein, 12 aa, artificial sequence
  Vi antigen peptide
PGHHDFVGLHHL

SEQ ID NO: 72 - protein, 12 aa, artificial sequence
  Vi antigen peptide
ENHYPVNIAHKL

SEQ ID NO: 73 - protein, 12 aa, artificial sequence
  Vi antigen peptide
DNHSPVNIAHKL

SEQ ID NO: 74 - protein, 12 aa, artificial sequence
  H5N1 peptide sequence
YINPHMYWMSVA

SEQ ID NO: 75 - protein, 12 aa, artificial sequence
  H5N1 peptide sequence
HTPPPQPYRTHI

SEQ ID NO: 76 - protein, 12 aa, artificial sequence
  H5N1 peptide sequence
TFWVQTAKPNPL

SEQ ID NO: 77 - protein, 12 aa, artificial sequence
  H5N1 peptide sequence
GHPSKTSGHPLT

SEQ ID NO: 78 - protein, 12 aa, artificial sequence
  H5N1 peptide sequence
TYVNIVLYDDVE

SEQ ID NO: 79 - protein, 12 aa, artificial sequence
  H5N1 peptide sequence
TTNFLNHAIAHK

SEQ ID NO: 80 - protein, 12 aa, artificial sequence
  H5N1 peptide sequence
YYNPSPPNPRTQ

SEQ ID NO: 81 - protein, 12 aa, artificial sequence
  H5N1 peptide sequence
TESPQYIALSFH

SEQ ID NO: 82 - protein, 12 aa, artificial sequence
  H5N1 peptide sequence
HWYDWLTRYSHL

SEQ ID NO: 83 - protein, 12 aa, artificial sequence
  H5N1 peptide sequence
ATYTTDAQSYHM

SEQ ID NO: 84 - protein, 12 aa, artificial sequence
  H5N1 peptide sequence
DHYWHRSNTLSH

SEQ ID NO: 85 - protein, 12 aa, artificial sequence
  H5N1 peptide sequence
VTSHDLKKSGTW

SEQ ID NO: 86 - protein, 12 aa, artificial sequence
  H5N1 peptide sequence
WEFAYKNTRYYW

SEQ ID NO: 87 - protein, 12 aa, artificial sequence
  H5N1 peptide sequence
SWTSLPLHEAIH

SEQ ID NO: 88 - protein, 12 aa, artificial sequence
  H5N1 peptide sequence
TLAHTHTSTSSF

SEQ ID NO: 89 - protein, 12 aa, artificial sequence
  H5N1 peptide sequence
WHWSFFASPLPA

SEQ ID NO: 90 - protein, 12 aa, artificial sequence
  H5N1 peptide sequence
WHWNARNWSSQQ

SEQ ID NO: 91 - protein, 12 aa, artificial sequence
  H5N1 peptide sequence
CWTSLPLHEAIH

SEQ ID NO: 92 - protein, 12 aa, artificial sequence
  H5N1 peptide sequence
VPTECSGRTSCT

SEQ ID NO: 93 - protein, 12 aa, artificial sequence
  H5N1 peptide sequence
WSNHWWHSKWAI

SEQ ID NO: 94 - protein, 12 aa, artificial sequence
  H5N1 peptide sequence
HIWNWSNWTQWT

SEQ ID NO: 95 - protein, 12 aa, artificial sequence
  H5N1 peptide sequence
HIFHNTHWWQRW

SEQ ID NO: 96 - protein, 12 aa, artificial sequence
  H5N1 peptide sequence
TNYDYIPDTQNT

SEQ ID NO: 97 - protein, 32 aa, artificial sequence
  H5N1 peptide sequence
SWSSHSNSTPTSYNTNQTQNPTSTSTNQPNNN

SEQ ID NO: 98 - protein, 36 aa, artificial sequence
  H5N1 peptide sequence
NHEKIPKSSWSSHWKYNTNQEDNKTIKPNDNEYKVK

SEQ ID NO: 99 - protein, 12 aa, artificial sequence
  CD147 peptide sequence
YPHFHKHTLRGH

SEQ ID NO: 100 - protein, 12 aa, artificial sequence
  CD147 peptide sequence
YPHFHKHSLRGQ

SEQ ID NO: 101 - protein, 12 aa, artificial sequence
  CD147 peptide sequence
DHKPFKPTHRTL

SEQ ID NO: 102 - protein, 12 aa, artificial sequence
  CD147 peptide sequence
FHKPFKPTHRTL

SEQ ID NO: 103 - protein, 12 aa, artificial sequence
  CD147 peptide sequence
QSSCHKHSVRGR

SEQ ID NO: 104 - protein, 12 aa, artificial sequence
  CD147 peptide sequence
QSSFSNHSVRRR

SEQ ID NO: 105 - protein, 12 aa, artificial sequence
  CD147 peptide sequence
DFDVSFLSARMR

SEQ ID NO: 106 - protein, 8 aa, artificial sequence
  Schistosoma mansoni peptide sequence
VLLRRIGG

SEQ ID NO: 107 - protein, 8 aa, artificial sequence
  Schistosoma mansoni peptide sequence
HLLRLSEI

SEQ ID NO: 108 - protein, 8 aa, artificial sequence
  Schistosoma mansoni peptide sequence
SLLTYMKM

SEQ ID NO: 109 - protein, 8 aa, artificial sequence
  Schistosoma mansoni peptide sequence
YLLQKLRN

SEQ ID NO: 110 - protein, 8 aa, artificial sequence
  epitope
SGGGSGGG

SEQ ID NO: 111 - protein, 4 aa, artificial sequence
  eiptope
SGGG

SEQ ID NO: 112 - protein, 7 aa, artificial sequence
  eiptope
SGGSSGG

SEQ ID NO: 113 - protein, 14 aa, artificial sequence
  reactive effector
SGGGQLCPYELWEL

SEQ ID NO: 114 - protein, 6 aa, artificial sequence
  reactive effector
CHGGGS

SEQ ID NO: 115 - protein, 45 aa, artificial sequence
  BJAB aptamer sequence
CACTGGGTGGGGTTAGCGGGCGATTTAGGGATCTTGAGTGGTGGA

SEQ ID NO: 116 - protein, 100 aa, artificial sequence
  BJAB aptamer sequence
CACTGGGTGGGGTTAGCGGGCGATTTAGGGATCTTGAGTGGTGTCAAAAGCCAAAAAGCCACTGTGTCCTGAAGAAAGCAAAGACATCTGGACAAAAAGC

SEQ ID NO: 117 - protein, 5 aa, artificial sequence
  epitope
SQRRL

SEQ ID NO: 118 - protein, 5 aa, artificial sequence
  epitope
QTGAR

SEQ ID NO: 119 - DNA, 31 bp, artificial sequence
  DNA splint
tccagatgtctttgctttcttcaggacacag

SEQ ID NO: 120 - DNA, 85 bp, artificial sequence
  Right aptamer
gcaaagacatctggacacgccacttatagtctacgtgaagcactgcgctggaacagcctaaaaaaggagaaggagacttagaggc

SEQ ID NO: 121 - DNA, 28 bp, artificial sequence
  Oligonucleotide complementary to 3 end of #207
tgtaggactctagatcggaagttgtagc

SEQ ID NO: 122 - DNA, 28 bp, artificial sequence
  Oligonucleotide complementary to 5 end of #208
ctcgaaggctacgtgctagcgcatacat

SEQ ID NO: 123 - DNA, 31 bp, artificial sequence
  L/R inter aptamer region
uccagaugucuuugcuuucuucaggacacag

SEQ ID NO: 124 - DNA, 20 bp, artificial sequence
  Forward R primer
gcaaagacatctggacacgc

SEQ ID NO: 125 - DNA, 21 bp, artificial sequence
  Reverse R primer
gcctctaagtctccttctcct

SEQ ID NO: 126 - protein, 10 aa, artificial sequence
  Melan A MART epitope
ELAGIGILTV

SEQ ID NO: 127 - DNA, 56 bp, artificial sequence
  human papillomavirus HPV sequence
taactgtcaaaagccactgtgtcctgaagaaaagcaaagacatctggacaaaaagc

SEQ ID NO: 128 - DNA, 56 bp, artificial sequence
  scrambled human papillomavirus HPV sequence
tagcgcaaataagccgccagaacgatgatataaacagcattaggtaagctacaaca

SEQ ID NO: 129 - DNA, 30 bp, artificial sequence
  oligonucleotide
tccagatgtctttgctttcttcaggacaca

SEQ ID NO: 130 - DNA, 17 bp, artificial sequence
  oligonucleotide
gctgtgtcctgaagaaa

SEQ ID NO: 131 - DNA, 17 bp, artificial sequence
  oligonucleotide
tttcttcaggacacagc

SEQ ID NO: 132 - protein, 15 aa, artificial sequence
  linker sequence
CSGGGQLGPYELGGS

SEQ ID NO: 133 - protein, 13 aa, artificial sequence
  linker sequence
SGGWELSHSGGGC